(19) United States
(12) Patent Application Publication
(10) Pub. No.: US 2023/0159892 A1
(43) Pub. Date: May 25, 2023

(54) ENGINEERED IMMUNE CELLS

(71) Applicant: Synthekine, Inc., Menlo Park, CA (US)

(72) Inventors: Paul-Joseph PENAFLOR ASPURIA, Menlo Park, CA (US); Martin OFT, Menlo Park, CA (US)

(73) Assignee: Synthekine, Inc., Menlo Park, CA (US)

(21) Appl. No.: 17/916,974

(22) PCT Filed: Apr. 6, 2021
(86) PCT No.: PCT/US21/26067

§ 371 Date: Oct. 4, 2022

Related U.S. Application Data
(60) Provisional application No. 63/006,065, filed on Apr. 6, 2020.

(51) Int. Cl. C12N 5/0783; C07K 14/47; C12N 15/86

(52) U.S. Cl. CPC C12N 5/0636; C07K 14/4748; C12N 15/86; C07K 2319/03

(57) ABSTRACT

The present disclosure is directed to a recombinantly modified immune cell comprising a nucleic acid sequence encoding a receptor comprising an orthogonal extracellular domain and a nucleic acid sequence encoding an orthogonal ligand that exhibits specific binding for the orthogonal extracellular domain of the receptor.

### CROSS-REFERENCE TO RELATED PATENT APPLICATIONS

The present patent application is a 371 U.S. National Phase of PCT International Application No. PCT/US2021/026067, filed Apr. 6, 2021, which claims benefit of priority to U.S. Provisional Patent Application No. 63/006,065, filed Apr. 6, 2020, which is incorporated by reference for all purposes.

### SEQUENCE LISTING

The instant application contains a Sequence Listing which has been submitted electronically in ASCII format and is hereby incorporated by reference in its entirety. Said ASCII copy, created on Oct. 4, 2022, is named 106249_1350253_SEQ_LST.txt and is 68,498 bytes in size.

### BACKGROUND OF THE INVENTION

The controlled manipulation of the differentiation, development and proliferation of cells, particularly engineered immune cells, is of significant clinical interest. A variety of immune cells have been engineered for use in therapeutic applications such as the recognition and killing of cancer cells, intracellular pathogens and cells involved in autoimmunity. The use of engineered cell therapies in the treatment of cancer is facilitated by the selective activation and expansion of engineered cells (such as T cells) to provide specific functions and are directed to selectively attack cancer cells. In some examples of adoptive immunotherapy, T cells are isolated from the blood or tumor tissue of a subject, processed ex vivo, and re-infused into the subject. Compositions and methods that enable selective activation and expansion of such engineered cell populations are therefore desirable.

A challenge to the preparation of cell therapy products is that such “living drugs” require close control of their environment to preserve viability and functionality. In practice, isolated cells, whether derived from a patient (autologous) or from a single donor source (allogeneic), begin to lose function rapidly following removal from a subject or the controlled culture conditions. Successful maintenance of the viability of isolated cells while outside the subject or controlled culture conditions enables the isolated cells to return to functionality for reinsertion into the cell product manufacturing workflow or into patients. Additionally, successful maintenance of the viability of the engineered cells following administration of the engineered cells to a subject facilitates the clinical response to such cell therapy.

One challenge to the clinical application of engineered cell therapies is to maintain the viability of engineered cells once they are administered to a subject so as to maximize their therapeutic effectiveness. For example, in the case of the clinical applications of engineered T cells (e.g. CAR-T cells) the common means to maintain the viability of the engineered cells following administration to the subject is the systemic administration of the pluripotent cytokine interleukin-2, usually in the form of aldesleukin (Proleukin®), a human IL2 analog having a C125S substitution. In typical clinical practice of adoptive cell therapy with TILs or CAR-Ts, shortly after infusion of the TILS or CAR-T cells, the subject receives intravenous “high-dose” IL2 (720,000 IU/kg every 8 h) for as long as the subject can tolerate the treatment. The administration of IL2 is thought to enhance the survival and clinical efficacy of the cell product.

However, the systemic administration of IL2 is associated with non-specific stimulatory effects beyond the population of engineered cells and is associated, particularly in high doses, with significant toxicity in human subjects. The effect of high dose IL2 typically used in supportive regimens is documented to result in significant toxicities. The most prevalent side effects observed from the use of IL2 supportive therapy following adoptive cell transfer (ACT) include chills, high fever, hypotension, oliguria, and edema due to the systemic inflammatory and capillary leak syndrome as well as reports of autoimmune phenomena such as vitiligo or uveitis. Furthermore, the clinically approved form of IL2 (e.g. Proleukin®) possesses a short lifespan in vivo (on the order of hours) which requires that the IL2 be dosed frequently to maintain sufficient exposure of the engineered T cells to the administered IL2 to maintain the cells in an activated state.

Although cells from the administration of an adoptive cell therapy regimen may be detectable for months or even years following the administration of the cell product, a significant fraction (the majority) of the administered cells lapse into a quiescent state in which they lose therapeutic efficacy. Such loss of activity of the adoptively transferred cells frequently correlates with a loss of clinical efficacy including relapse or recurrence of the neoplastic disease. Consequently, challenge in cell-based therapies is to confer a desired regulatable behavior into the transferred cells that is protected from endogenous signaling pathways, that exhibits minimal cross reactivity with non-targeted endogenous cells, and that can be controlled selectively following administration of the engineered cell population to a subject.

CD122 is a component of the intermediate and high affinity IL2 receptor complexes. Sockolosky, et al. (Science (2018) 359: 1037-1042) and Garcia, et al. (United States Patent Application Publication US2018/0228841A1 published Aug. 16, 2018) describe an orthogonal IL2/CD122 ligand/receptor system to facilitate selective stimulation of cells engineered to express the orthogonal CD122. The present patent application incorporates by reference the disclosures of WO 2019/104092 and US 2018-0228842 A1) in their entireties. The contact of engineered T cells that express the orthogonal CD122 with a corresponding orthogonal ligand cognate for such orthogonal CD122 (“IL2 ortholog”) facilitates specific activation of such engineered T cells that express the orthogonal CD122. In particular this orthogonal IL2 receptor ligand complex provides for selective expansion of cells engineered to express the orthogonal receptor in a mixed population of cells, in one embodiment, a mixed population of immune cells (e.g. T cells).

When employing cells expressing the orthogonal ligand system described above involves the use of a two-component system: (1) a cell expressing the orthogonal receptor and (2) contacting the cell expressing the orthogonal receptor with a cognate ligand for the ECD of the orthogonal receptor. In some embodiments, for example in the treatment of solid tumors, it would be desirable to maintain the selective expansion of the engineered immune cell conferred by the orthogonal receptor/ligand system without the requirement of administration of an independent activating ligand. For example, in contrast to the treatment of hematological malignancies, the treatment of solid tumors with engineered cells expressing the orthogonal ligand, the cell expressing the orthogonal ligand may distant from or poorly served by the blood supply and thus the supply of the orthogonal ligand. The present disclosure addresses this issue by providing engineered cells expressing an orthogonal receptor which are capable of autonomous activation and regulation.

### SUMMARY OF THE INVENTION

The present disclosure is directed to a recombinantly modified immune cell comprising a nucleic acid sequence encoding a receptor comprising an orthogonal extracellular domain and a nucleic acid sequence encoding an orthogonal ligand that exhibits specific binding for the orthogonal extracellular domain of the receptor.

In one embodiment, the present disclosure provides a recombinantly modified mammalian cell comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

In some embodiments, the recombinantly modified mammalian cell is a recombinantly modified immune cell. In some embodiments, the recombinantly modified immune cell is stem cell or a cell of lymphoid origin including but not limited to B cells, T cells, Natural Killer (NK) cells, NKT cells, cytotoxic T lymphocytes (CTLs), regulatory T cells (Tregs), dendritic cells, killer dendritic cells, and mast cells. inflammatory T-lymphocytes, cytotoxic T-lymphocytes, regulatory T-lymphocytes or helper T-lymphocytes including tumor infiltrating lymphocytes (TILs), CD4+ T-lymphocytes and CD8+ T-lymphocytes, cytotoxic T lymphocytes (CTLs), a regulatory T cell (Tregs), including subsets of CD8+ T lymphocytes of various phenotypes including T effector memory phenotype (Tem), T central memory phenotype (Tcm), terminally differentiated Tcm and Tem cells that express CD45RA (Temra), tissue resident memory (Trm) cells, and peripheral memory (Tpm) cells.

In some embodiments the recombinantly modified mammalian cell is a targeting redirected immune cell. By targeting redirected immune cell in the context of the present disclosure as a recombinantly modified immune cell that expresses a non-native molecule on the surface of the cell, the non-native molecule exhibiting specific binding for molecule on the surface of a second cell so that the modified immune cell now binds to the second cell by virtue of the action of the non-native cell surface molecule expressed on the immune cell. In many instances, the non-native surface molecule is an antibody or antibody fragment (scFv) that has specific binding for a tumor antigen expressed on a second (tumor) cell such that the modified immune cell binds to the tumor cell for which the immune cell would otherwise have low affinity. Examples of such targeting redirected immune cells include but are not limited to CAR-T cells and TCR-engineered cells. In some embodiments the immune cell is a CAR-T cell wherein the targeting domain of the CAR-T cell exhibits specific binding to a tumor antigen of a hematopoietic or solid tumor cell. Example of such targeting domains include antibodies, as discussed below, that exhibit specific binding to one or more tumor antigens selected from the group consisting of CD19, CD20, CD22, ROR1, CD4, CD7, CD38, CD30, B-cell maturation antigen, Lewis Y antigen, mesothelin, EGFR, GPC3, MUC1, HER2, GD2, CEA, EpCAM, PSCA, PSMA, IL3Ra2, EGFRvIII, CAIX, c-Met, and TAG72.

In some embodiments, the recombinantly modified immune cell is further modified to express at least one drug resistance gene, the drug resistance gene operably linked to an expression control sequence operable in the immune cell.

In another embodiment, the present disclosure provides a recombinant vector encoding: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2. In some embodiments, the first and second nucleic acid sequence are operably linked to an expression control sequence operable in the target recombinantly modified immune cell such that expression of the first and second nucleic acid sequences are under control of a single expression control sequence (i.e. a bicistronic expression cassette). In some embodiments, the first and second nucleic acid sequence are operably linked to an expression control sequence operable in the target recombinantly modified immune cell such that expression of the first and second nucleic acid sequences are under control of a single expression control sequence and the first and second nucleic acid sequences are linked by nucleic acid sequence corresponding to mRNA a ribosome skipping site such as the picornaviral 2A sequence (See, e.g. Funston, et al. (2008) Journal of General Virology 89:389-396) or internal ribosome entry site (IRES) sequence. In some embodiments, when the immune cell is a CAR-T cell, the vector may be polycistronic encoding the CAR, the nucleic acid sequence encoding the orthogonal receptor and the orthogonal ligand are under the control of a single expression control sequence. In other embodiments, one or more of the nucleic acid sequences may be under the control of separate expression control sequences.

In some embodiments, the first and second nucleic acid sequence are operably linked to individual expression control sequences, such sequences each being operable in the recombinantly modified immune cell such that expression of the first and second nucleic acid sequences are under control of a separate expression control sequence. In some embodiments, the first and second nucleic acid sequence are operably linked to individual expression control sequences, such sequences each operable in the target recombinantly modified immune cell such that expression of the first and second nucleic acid sequences are under control of a separate expression control sequence wherein the first and second expression control sequences are different.

In some embodiments, the expression control sequence is sleeted from the group consisting of constitutively active, selectively active, and regulated expression control sequences as more fully described hereinbelow.

The present disclosure further provides vectors comprising the nucleic acids encoding the hoCD122 ECD receptor and the hoIL2 ligand and associated expression control sequences and nucleic acid molecules encoding functions that are desired in the engineered immune cell such as drug resistance genes, targeting ligands, chimeric antigen receptor sequences, engineered TCR sequences, etc.

In another embodiment, the expression vector may be a viral vector. When a viral vector system is to be employed for CAR and expression of the orthogonal receptor, and/or ligand retroviral or lentiviral expression vectors are preferred. In particular, the viral vector is a gamma retrovirus (Pule, et al. (2008) Nature Medicine 14(11):1264-1270), self-inactivating lentiviral vectors (June, et al. (2009) Nat Rev Immunol 9(10):704-716) and retroviral vectors as described in Naldini, et al. (1996) Science 272: 263-267; Naldini, et al. (1996) Proc. Natl. Acad. Sci. USA Vol. 93, pp. 11382-11388; Dull, et al. (1998) J. Virology 72(11):8463-8471; Milone, et al. (2009) 17(8):1453-1464; Kingsman, et al. U.S. Pat. No. 6,096,538 issued Aug. 1, 2000 and Kingsman, et al. U.S. Pat. No. 6,924,123 issued Aug. 2, 2005. In one embodiment of the invention, the CAR expression vector is a Lentivector® lentiviral vector available from Oxford Biomedica.

In some embodiments, the vector is a viral vector. In some embodiments the viral vector is selected from the group consisting of retroviral vectors and lentiviral vectors.

In some embodiments, the present disclosure provides recombinantly modified cells expressing orthogonal receptors, the orthogonal receptor having an extracellular domain that specifically binds to a cognate orthogonal ligand, a transmembrane domain and an intracellular domain. In some embodiments, the orthogonal receptor is a receptor comprising an ECD of FORMULA #1. In some embodiments, the orthogonal receptor comprising an ECD having an amino acid sequence of SEQ ID NO. 1. In some embodiments, the orthogonal receptor comprises the extracellular domain of FORMULA #1 and an intracellular domain (ICD) corresponding to the ICD of hCD122. In some embodiments, the orthogonal receptor comprises the extracellular domain of FORMULA #1 and the transmembrane and intracellular domains (ICD) corresponding to the transmembrane and ICD domains of hCD122. In some embodiments the orthogonal receptor comprises the amino acid sequence of SEQ ID NO. 2. In some embodiments, the intracellular domain of the orthogonal receptor further comprises at least one STAT3 binding motif.

In some embodiments, the present disclosure provides recombinantly modified cells comprising at least one nucleic acid sequences encoding an hIL2 ortholog wherein said at nucleic acid is encoded by a viral vector. In some embodiments, the orthologs are human IL2 orthologs. In some embodiments, the IL2 orthologs are ligands for a receptor comprising an ECD of FORMULA #1. In some embodiments, the hIL2 orthologs are ligands for an orthogonal receptor comprising the extracellular domain of CD122 comprising amino acid substitutions at positions H133 and Y134. In some embodiments, the IL2 orthologs are ligands for an orthogonal receptor comprising the extracellular domain of CD122 comprising amino acid substitutions at positions H133 and Y134. In some embodiments, the IL2 orthologs are ligands for an orthogonal hCD122 comprising amino acid substitutions at positions H133 and Y134. In some embodiments, the IL2 orthologs are ligands for an orthogonal hCD122 comprising the amino acid substitutions H133D and Y134F (SEQ ID NO: 1).

In some embodiments, the present disclosure provides immune cells that are recombinantly modified to express an orthogonal receptor. In some embodiments, the present disclosure provides mammalian cells that are recombinantly modified to express a receptor comprising the ECD of an orthogonal CD122. In some embodiments, the present disclosure provides mammalian cells that are recombinantly modified to express an orthogonal receptor comprising the extracellular domain of CD122 comprising amino acid substitutions at positions H133 and Y134. In some embodiments, the present disclosure provides mammalian cells that are recombinantly modified to express an receptor comprising the extracellular domain of orthogonal hCD122 comprising amino acid, said ECD comprising the substitutions at positions H133D and Y134F (SEQ ID NO:1). In some embodiments, the present disclosure provides mammalian cells that are recombinantly modified to express an orthogonal hCD122 comprising the amino acid substitutions H133D and Y134F (SEQ ID NO:2).

In some embodiments, the present disclosure provides a method of causing a proliferative response in a mammalian immune cell, the cell having been recombinantly modified to express: (1) a receptor protein comprising an intracellular domain, a transmembrane domain and an extracellular domain (ECD), the extracellular domain comprising a polypeptide of the of FORMULA #1, and (2) a polypeptide ligand for the receptor protein comprising the (a) a secretion leader sequence (signal sequence) and (b) a polypeptide comprising the amino acid sequence of FORMULA #2 such that the polypeptide of the FORMULA #2 is secreted from the engineered immune cell and is capable of contacting and specifically binding to the ECD of the receptor protein, and by such contacting, activates the signaling cascade of the ICD of the receptor.

In some embodiments, the present disclosure provides a method of causing a proliferative signaling response in a mammalian immune cell, the cell having been recombinantly modified to express: (1) a receptor protein comprising an intracellular domain, a transmembrane domain and an extracellular domain (ECD), the extracellular domain comprising a polypeptide of the of FORMULA #1; and (2) a polypeptide ligand for the ECD of the receptor protein comprising, the polypeptide ligand comprising a fusion protein of membrane anchoring sequence and polypeptide comprising the amino acid sequence of FORMULA #2 such that a polypeptide comprising the amino acid sequence of of the FORMULA #2 is displayed (tethered) on the surface from the engineered immune cell. When the fusion protein comprising the polypeptide ligand is displayed (tethered) to the surface of the engineered mammalian immune cell, the ligand domain of the fusion protein is capable of contacting and specifically binding to the ECD of the receptor protein and activates the signaling cascade defined by of the ICD of the receptor. Sequences useful in the construction of the fusion protein to achieve tethered surface display of the polypeptide ligand of FORMULA #2 are known in the art.

The present disclosure further provides a method activation of proliferation of a mammalian immune cell expressing a receptor comprising an orthogonal ECD of FORMULA #1 with an effective amount of an ortholog of FORMULA #2. In some embodiments, for example where the expression orthogonal ligand encoded by the engineered cell is under the control of expression control sequences that are activated preferentially in vivo (e.g. where the nucleic acid sequence encoding ligand of FORMULA #2 is under control of a promoter activated in response to the tumor microenvironment), it may be desirable to contact the engineered the cells ex vivo (e.g. in preparing the cell product for administration to ex vivo stimulation or proliferation) or in vivo (e.g. contemporaneously with and for period of time after administration of the engineered cell product) with a hIL2 ortholog polypeptide of the FORMULA #2 to facilitate the administration of an activated engineered cell population and support the administered cell population in he subject for a period of time after administration ensure sufficient proliferative signaling for the engineered immune cells before they begin (or “ramp up”) autonomous expression of the orthogonal ligand and concomitant autonomous activation of the engineered immune cell expressing the receptor comprising the orthogonal ECD comprising the amino acid sequence of FORMULA #1.

The present disclosure further provides a method of effecting a response in a mammalian immune cell expressing a receptor comprising an orthogonal ECD of FORMULA #1 by contacting said immune cell with an effective amount of a cognate ortholog of FORMULA #2 ex vivo and/or in vitro in an amount sufficient to effect intracellular signaling from the ICD of the receptor of SEQ ID NO:2. The present disclosure provides a method of causing a response in a mammalian immune cell expressing a receptor of SEQ ID NO:2, the method comprising contacting said receptor with an effective amount of a polypeptide of FORMULA #2, wherein said method is practiced ex vivo.

The present disclosure further provides a method of causing a response in a mammalian immune cell expressing a receptor of SEQ ID NO:2, the method comprising contacting said receptor with an effective amount of a polypeptide of FORMULA #2, wherein said method is practiced in vivo by contacting the cell with in vivo in an amount sufficient to cause a response in the recombinantly modified immune cell.

In some embodiments, the present disclosure provides methods of use comprising the use a first hIL2 ortholog of FORMULA #2 (i.e., orthogonal hIL2 ligand) ex vivo and a second hIL2 ortholog of FORMULA #2 in vivo. In some embodiments, the present disclosure provides methods of use comprising the use a first hIL2 ortholog of FORMULA #2 ex vivo and a second hIL2 ortholog of FORMULA #2 in vivo, wherein the first hIL2 ortholog of FORMULA #2 and the second hIL2 ortholog of FORMULA #2 are the same orthologs or different orthologs. In some embodiments, the present disclosure provides methods of use of hIL2 orthologs of FORMULA #2 to cause the proliferation of a mammalian immune cell expressing a receptor comprising an orthogonal ECD of FORMULA #1. In some embodiments, the present disclosure provides methods of use of hIL2 orthologs of FORMULA #2 to cause the activation of a mammalian cell expressing a mammalian immune cell expressing a receptor comprising an orthogonal ECD of FORMULA #1. In some embodiments, the present disclosure provides methods of use of hIL2 orthologs of FORMULA #2 ex vivo and/or in vivo to cause the proliferation of a mammalian immune cell expressing a receptor comprising an orthogonal ECD of FORMULA #1. In some embodiments, the present disclosure provides methods of use of hIL2 orthologs of FORMULA #2 to cause the activation of a mammalian cell recombinantly modified to express an orthogonal receptor comprising the extracellular domain of hCD122 comprising amino acid substitutions at of SEQ ID NO: 1. In some embodiments, the present disclosure provides methods of use of hIL2 orthologs of FORMULA #2 to cause the activation of a mammalian cell recombinantly modified to express an orthogonal receptor comprising the amino acid substitutions H133D and Y134F (SEQ ID NO:2).

In some embodiments, the present disclosure provides methods for the preparation of a population of recombinantly modified mammalian immune cells, the immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

The present disclosure further provides methods for the preparation of a population of recombinantly modified mammalian immune cells, said immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2, and nucleic acid sequence encoding a chimeric antigen receptor.

The present disclosure further provides a cell therapy product comprising a pharmaceutically acceptable formulation of population of recombinantly modified mammalian immune cells, said immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

The present disclosure further provides a cell therapy product comprising a pharmaceutically acceptable formulation of population of recombinantly modified mammalian immune cells, said immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2, and nucleic acid sequence encoding a chimeric antigen receptor.

The present disclosure further provides methods of preparing a pharmaceutically acceptable dosage form of a cell therapy product comprising at least one (alternatively 2, 3, 4 or more) species of engineered immune cells that express a transmembrane receptor protein wherein the extracellular domain of such transmembrane receptor protein comprises the extracellular domain of an CD122 orthogonal polypeptide of FORMULA #1, a secreted form of an IL2 ligand of FORMULA #2, and a chimeric antigen receptor, wherein the fraction of engineered cells in the cell therapy product comprises at least 30%, alternatively at least 40%, alternatively at least 50%, alternatively at least 60%, alternatively at least 70%, alternatively at least 80%, or alternatively at least 90% of the total number of cells in the cell therapy product.

In some embodiments a therapeutic method is provided, the method comprising introducing into a subject in need thereof of pharmaceutically acceptable formulation comprising a population of engineered allogenic or autologous immune cells allogeneic that express: (1) a transmembrane receptor polypeptide wherein the extracellular domain of the transmembrane receptor polypeptide comprises the extracellular domain of an CD122 orthogonal polypeptide of FORMULA #1; (2) a secreted or membrane tethered form of an IL2 ligand of FORMULA #2; and (3) a chimeric antigen receptor.

In some embodiments, the compositions and methods of the present disclosure comprise the step of genetically modifying a human immune cell by using at least one endonuclease to facilitate incorporate the modifications of to the ECD of the orthogonal hCD122 of FORMULA #1 into the genomic sequence of the human immune cell. As used herein, the term “endonuclease” is used to refer to a wild-type or variant enzyme capable of catalyzing the cleavage of bonds between nucleic acids within a DNA or RNA molecule, preferably a DNA molecule. The endonucleases of the present disclosure are sequence specific in that they recognize and cleave the nucleic acid molecules at specific “target” sequences. Endonucleases are often categorized with respect to the degree of specificity and sequence identity characteristic of the target sequences. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogeneous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23(3):967-973, a TALE-nuclease, a Cas9 endonuclease from CRISPR system as or a modified restriction endonuclease to extended sequence specificity (Eisenschmidt, et al. 2005; 33(22): 7039-7047). In some embodiments of the invention, the immune cell (e.g. a CAR-T expressing the orthogonal receptor ECD of FORMULA #1) is modified to reduce alloreactivity through inactivation of one more components of the T-cell receptor (TCR). Methods for such modification of T cells is described in Galetto, et al. United States Patent Application Publication No. US 2013/015884A1 published Nov. 28, 2013 and methods for TCRalpha deficient T-cells by expressing pTalpha resulting in restoration of a functional CD3 complex as described in Galetto, et al. U.S. Pat. No. 10,426,795B2 issued Oct. 21, 2019. the teaching of which is herein incorporated by reference. In one embodiment, the immune cell has at least one CD122 allele converted into a nucleic acid sequence encoding orthogonal hCD122 of with an ECD of FORMULA #1 or SEQ ID NO: 2. In an alternative embodiment, the immune cell has both CD122 alleles converted into a nucleic acid sequence encoding orthogonal hCD122 of with an ECD of FORMULA #1 or SEQ ID NO: 2 such that the immune cell does not express a wild-type CD122 receptor making the proliferation of such cell dependent on the on the supply of an orthogonal ligand of FORMULA #2.

### DETAILED DESCRIPTION

In order for the present disclosure to be more readily understood, certain terms and phrases are defined below as well as throughout the specification. The definitions provided herein are non-limiting and should be read in view of the knowledge of one of skill in the art would know.

Before the present methods and compositions are described, it is to be understood that this invention is not limited to particular method or composition described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); aa=amino acid(s); kb=kilobase(s); nt=nucleotide(s); pg=picogram; ng=nanogram; pg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; PCR=polymerase chain reaction; NHS=N-hydroxysuccinimide; HSA=human serum albumin; MSA=mouse serum albumin; DMEM=Dulbeco's Modification of Eagle's Medium; GC=genome copy; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Unless otherwise indicated, the following terms are intended to have the meaning set forth below. Other terms are defined elsewhere throughout the specification.

Activate: As used herein the term “activate” is used in reference to a receptor or receptor complex to reflect the biological effect of the binding of an agonist ligand to the receptor. For example, it is said that the binding of IL2 ligand to the IL2 receptor “activates” the signaling of the receptor to produce one or more intracellular biological effects (e.g. phosphorylation of STAT5).

Activity: As used herein, the term “activity” is used with respect to a molecule to describe a property of the molecule with respect to a test system or biological function such as the degree of binding of the molecule to another molecule. Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a biological activity per unit of administered agent such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. The term “proliferative activity” encompasses an activity that promotes cell division including dysregulated cell division as that observed in neoplastic diseases, inflammatory diseases, fibrosis, dysplasia, cell transformation, metastasis, and angiogenesis.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g. an ortholog, an IL2 ortholog, a CAR-T cell, a chemotherapeutic agent, an antibody, or a pharmaceutical formulation comprising one or more of the foregoing). Administration of an agent may be achieved through any of a variety of art recognized methods including but not limited to the topical, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), respiratory inhalers, intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell.

Adverse Event: As used herein, the term “adverse event” refers to any undesirable experience associated with the use of a therapeutic or prophylactic agent in a subject. Adverse events do not have to be caused by the administration of the therapeutic or prophylactic agent (e.g. the IL2 ortholog) but may arise from unrelated circumstances. Adverse events are typically categorized as mild, moderate, or severe. As used herein, the classification of adverse events as used herein is in accordance with the Common Terminology Criteria for Adverse Events v4.03 (CTCAE) dated Jun. 14, 2010 published by the United States Department of Health and Human services, National Institutes of Health National Cancer Institute.

Affinity: As used herein the term “affinity” refers to the degree of specific binding of a first molecule (e.g. a ligand) to a second molecule (e.g. a receptor) and is measured by the binding kinetics expressed as K_{d}, a ratio of the dissociation constant between the molecule and the its target (K_{off}) and the association constant between the molecule and its target (Kₒₙ).

Agonist: As used herein, the term “agonist” refers an agent that specifically binds a second molecule (“target”) and interacts with the target to cause or promote an increase in the activation of the target. Agonists are activators that modulate cell activation, enhance activation, sensitize cells to activation by a second agent, or up-regulate, e. g., a gene, protein, ligand, receptor, biological pathway including an immune checkpoint pathway in a cell, or cell proliferation. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to full response (i.e. the response associated with the naturally occurring ligand/receptor binding interaction) induced by receptor under study, or a partial agonist. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an efficacy of more than 100%. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Antibody: As used herein, the term “antibody” refers collectively to: (a) glycosylated and non-glycosylated the immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule.

The term antibody is not restricted to immunoglobulins derived from any particular mammalian species and includes murine, human, equine, camelids, antibodies, human antibodies. The term antibody includes so called “heavy chain antibodies” or “VHHs” or “Nanobodies®” as typically obtained from immunization of camelids (including camels, llamas and alpacas (see, e.g. Hamers-Casterman, et al. (1993) Nature 363:446-448). Antibodies having a given specificity may also be derived from non-mammalian sources such as VHHs obtained from immunization of cartilaginous fishes including, but not limited to, sharks.

The term “antibody” encompasses antibodies isolatable from natural sources or from animals following immunization with an antigen and as well as engineered antibodies including monoclonal antibodies, bispecific antibodies, tri-specific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term antibody includes both the parent antibody and its derivatives such as affinity matured, veneered, CDR grafted, humanized, camelized (in the case of VHHs), or binding molecules comprising binding domains of antibodies (e.g. CDRs) in non-immunoglobulin scaffolds. The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries. In one embodiment, an “antibody” is a mammalian immunoglobulin. In some embodiments, the antibody is a “full length antibody” comprising variable and constant domains providing binding and effector functions. In most instances, a full-length antibody comprises two light chains and two heavy chains, each light chain comprising a variable region and a constant region. In some embodiments the term “full length antibody” is used to refer to conventional IgG immunoglobulin structures comprising two light chains and two heavy chains, each light chain comprising a variable region and a constant region providing binding and effector functions. The term antibody includes antibody conjugates comprising modifications to prolong duration of action such as fusion proteins or conjugation to polymers (e.g. PEGylated) as described in more detail below.

Biological Sample: As used herein, the term “biological sample” or “sample” refers to a sample obtained or derived from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, and an immunoglobulin enriched fraction derived from one or more of these tissues. In some embodiments, the sample is obtained from a subject who has been exposed to a therapeutic treatment regimen including a pharmaceutical formulation of a an IL2 ortholog, such as repeatedly exposed to the same drug. In other embodiments, the sample is obtained from a subject who has not recently been exposed to the IL2 ortholog or obtained from the subject prior to the planned administration of the IL2 ortholog.

CAR” or “Chimeric Antigen Receptor”: As used herein, the terms “chimeric antigen receptor” and “CAR” are used interchangeably to refer to a chimeric polypeptide comprising multiple functional domains arranged from amino to carboxy terminus in the sequence: (a) an extracellular domain (ECD) comprising an antigen binding domain (ABD) and “hinge” domain, (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs may be prepared in accordance with principles well known in the art. See e.g., Eshhar, et al. (U.S. Pat. No. 7,741,465 B1 issued Jun. 22, 2010); Sadelain, et al. (2013) Cancer Discovery 3(4):388-398; Campana and Imai (U.S. Pat. No. 8,399,645 issued Mar. 19, 2013) Jensen and Riddell (2015) Current Opinions in Immunology 33:9-15; Gross, et al. (1989) PNAS(USA) 86(24):10024-10028; Curran, et al. (2012) J Gene Med 14(6):405-15; Brogdon, et al. (U.S. Pat. No. 10,174,095 issued Jan. 8, 2019) Guedan, et al. (2019) Engineering and Design of Chimeric Antigen Receptors (2019) Molecular Therapy: Methods & Clinical Development Vol. 12: 145-156.

CAR-T Cell: As used herein, the terms “chimeric antigen receptor T-cell” and “CAR-T cell” are used interchangeably to refer to a T-cell that has been recombinantly modified to express a chimeric antigen receptor. In some embodiments, As used herein, a CAR-T cell may be engineered to express an CD122 ortho polypeptide (orthogonal CAR-T cells). Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

CD25: As used herein, the terms “CD25”, “IL2 receptor alpha”, “IL2Rα”, “IL2Ra” and “p55” are used interchangeably to the 55 kD polypeptide that is constitutively expressed in Treg cells and inducibly expressed on other T cells in response to activation and is also referred to in the literature as the “low affinity” IL2 receptor. Human CD25 nucleic acid and protein sequences may be found as Genbank accession numbers NM__000417 and NP_0004Q8 respectively. The human CD25 is expressed as a 272 amino acid pre-protein comprising a 21 amino acid signal sequence which is post-translationally removed to render a 251 amino acid mature protein. Amino acids 22-240 (amino acids 1-219 of the mature protein) correspond to the extracellular domain. Amino acids 241-259 (amino acids 220-238 of the mature protein) correspond to transmembrane domain. Amino acids 260-272 (amino acids 239-251 of the mature protein) correspond to intracellular domain. The amino acid sequence of the mature form of hCD25 (without the signal sequence) is:

(SEQ ID NO: 5)
ELCDDDPPEIPHATFKAMAYKEGTMLNCEC
KRGFRRIKSGSLYMLCTGNSSHSSWDNQCQ
CTSSATRNTTKQVTPQPEEQKERKTTEMQS
PMQPVDQASLPGHCREPPPWENEATERIYH
FVVGQMVYYQCVQGYRALHRGPAESVCKMT
HGKTRWTQPQLICTGEMETSQFPGEEKPQA
SPEGRPESETSCLVTTTDFQIQTEMAATME
TSIFTTEYQVAVAGCVFLLISVLLLSGLTW
QRRQRKSRRTI

CD122: As used herein, the terms “CD122”, “interleukin-2 receptor beta”, “IL2Rb”, “IL2β”, “IL15Rβ” and “p70-75” are used interchangeably to refer to the CD122 transmembrane protein. The human CD122 (hCD122) is expressed as a 551 amino acid protein, the first 26 amino acids comprising a signal sequence which is post-translationally cleaved in the mature 525 amino acid protein. Amino acids 27-240 (amino acids 1-214 of the mature protein) correspond to the extracellular domain, amino acids 241-265 (amino acids 225-239 of the mature protein) correspond to the transmembrane domain and amino acids 266-551 (amino acids 240-525 of the mature protein) correspond to the intracellular domain. As used herein, the term CD122 includes naturally occurring variants of the CD122 protein including the S57F and D365E (as numbered in accordance with the mature hCD122 protein). hCD122 is referenced at UniProtKB database as entry P14784. Human CD122 nucleic acid and protein sequences may be found as Genbank accession numbers NM_000878 and NP_000869 respectively. The amino acid sequence of the mature hCD122 protein without the signal sequence is:

(SEQ ID NO. 6)
AVNGTSQETCFYNSRANISCVWSQDGALQD
TSCQVHAWPDRRRWNQTCELLPVSQASWAC
NLILGAPDSQKLTTVDIVTLRVLCREGVRW
RVMAIQDFKPFENLRLMAPISLQVVHVETH
RCNISWEISQASHYFERHLEFEARTLSPGH
TWEEAPLLTLKQKQEWICLETLTPDTQYEF
QVRVKPLQGEFTTWSPWSQPLAFRTKPAAL
GKDTIPWLGHLLVGLSGAFGFIILVYLLIN
CRNTGPWLKKVLKCNTPDPSKFFSQLSSEH
GGDVQKWLSSPFPSSSFSPGGLAPEISPLE
VLERDKVTQLLLQQDKVPEPASLSSNHSLT
SCFTNQGYFFFHLPDALEIEACQVYFTYDP
YSEEDPDEGVAGAPTGSSPQPLQPLSGEDD
AYCTFPSRDDLLLFSPSLLGGPSPPSTAPG
GSGAGEERMPPSLQERVPRDWDPQPLGPPT
PGVPDLVDFQPPPELVLREAGEEVPDAGPR
EGVSFPWSRPPGQGEFRALNARLPLNTDAY
LSLQELQGQDPTHLV

and the amino acid sequence of the extracellular domain of the hCD122 is:

(SEQ ID NO. 7)
AVNGTSQETCFYNSRANISCVWSQDGALQD
TSCQVHAWPDRRRWNQTCELLPVSQASWAC
NLILGAPDSQKLTTVDIVTLRVLCREGVRW
RVMAIQDFKPFENLRLMAPISLQVVHVETH
RCNISWEISQASHYFERHLEFEARTLSPGH
TWEEAPLLTLKQKQEWICLETLTPDTQYEF
QVRVKPLQGEFTTWSPWSQPLAFRTKPAAL
GKDT

CD132: As used herein, the terms “CD132”, “IL2 receptor gamma”, “IL2Rg” and “IL2Rg” are used interchangeably to refer to a type 1 cytokine receptor and is shared by the receptor complexes for IL-4, IL-7, IL-9, IL-15, and IL21, hence the reference to the “common” gamma chain. Human CD132 (hCD132) is expressed as a 369 amino acid pre-protein comprising a 22 amino acid N-terminal signal sequence. Amino acids 23-262 (amino acids 1-240 of the mature protein) correspond to the extracellular domain, amino acids 263-283 (amino acids 241-262 of the mature protein) correspond to the 21 amino acid transmembrane domain, and amino acids 284-369 (amino acids 262-347 of the mature protein) correspond to the intracellular domain. hCD132 is referenced at UniProtKB database as entry P31785. Human CD132 nucleic acid and protein sequences may be found as Genbank accession numbers: NM_000206 and NP_000197 respectively. The amino acid sequence of the mature hCD132 protein is:

(SEQ ID NO. 8)
LNTTILTPNGNEDTTADFELTTMPTDSLSV
STLPLPEVQCFVFNVEYMNCTWNSSSEPQP
TNLTLHYWYKNSDNDKVQKCSHYLFSEEIT
SGCQLQKKEIHLYQTFVVQLQDPREPRRQA
TQMLKLQNLVIPWAPENLTLHKLSESQLEL
NWNNRFLNHCLEHLVQYRTDWDHSWTEQSV
DYRHKFSLPSVDGQKRYTFRVRSRFNPLCG
SAQHWSEWSHPIHWGSNTSKENPFLFALEA
VVISVGSMGLIISLLCVYFWLERTMPRIPT
LKNLEDLVTEYHGNFSAWSGVSKGLAESLQ
PDYSERLCLVSEIPPKGGALGEGPGASPCN
QHSPYWAPPCYTLKPET

CDRs: As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. In the context of the present disclosure, the numbering of the CDR positions is provided according to Kabat numbering conventions.

Circulating Tumor Cell: As used herein the term “circulating tumor cell (CTC)” refers to tumor cells shed from the tumor mass into peripheral circulation.

Comparable: As used herein, the term “comparable” is used to describe the degree of difference in two measurements of an evaluable quantitative or qualitative parameter. For example, where a first measurement of an evaluable quantitative parameter (e.g. the level of IL2 activity as determined by an CTLL-2 proliferation or phospho-STAT5 assay) and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Derived From: As used herein in the term “derived from”, in the context of an amino acid sequence or polynucleotide sequence (e.g., an amino acid sequence “derived from” an IL2 polypeptide), is meant to indicate that the polypeptide or nucleic acid has a sequence that is based on that of a reference polypeptide or nucleic acid (e.g., a naturally occurring IL2 polypeptide or an IL2-encoding nucleic acid), and is not meant to be limiting as to the source or method in which the protein or nucleic acid is made. By way of example, the term “derived from” includes homologs or variants of reference amino acid or DNA sequences.

Drug Resistance Gene: As used herein the term “drug resistance gene” refers to a nucleic acid sequence that encodes “resistance” to an agent, such as a chemotherapeutic agent (e.g. methotrexate). Expression of the drug resistance gene in the engineered cell permits proliferation of the cells in the presence of the agent to a greater extent than the proliferation of a corresponding cell without the drug resistance gene. The expression of the drug resistance gene in a cell permits proliferation of the cells in the presence of the agent and therefore facilitates combination with additional therapeutic agents, particularly agents which otherwise be toxic to the engineered cell. Examples of such drug resistance genes are drug resistance include genes that confer resistance of human cells to anti-metabolites, methotrexate, vinblastine, cisplatin, alkylating agents, anthracyclines, cytotoxic antibiotics, anti-immunophilins, their analogs or derivatives, and the like. n one embodiment, a drug resistance gene of the invention can confer resistance to a drug (or an agent), in particular an anti-cancer drug selected from aracytine, cytosine arabinoside, amsacrine, daunorubicine, idarubicine, novantrone, mitoxantrone, vepeside, etoposide (VP16), arsenic trioxyde, transretinoic acid, combination of arsenic trioxyde, transretinoic acid, mechlorethamine, procarbazine, chlorambucil, cytarabine, anthracyclines, 6-thioguanine, hydroxyurea, prednisone, and combination thereof. Examples of drug resistance genes that may be incorporated into the engineered immune cells of the present disclosure are well known to those of skill in the art.

Effective Concentration (EC): As used herein, the terms “effective concentration” or its abbreviation “EC” are used interchangeably to refer to the concentration of an agent (e.g., an IL2 ortholog) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect.

Enriched: As used herein in the term “enriched” refers to a sample that is non-naturally manipulated so that a species (e.g. a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., as in a recombinantly modified bacterial or mammalian cell). In some embodiments, the term “enriched” is used herein in reference to a population of cells comprising cells that express an orthogonal receptor following contacting the population of cells with cognate ortholog in an amount sufficient to cause a response in those cells that express an orthogonal receptor, the response being proliferation, such that concentration of cells that express the orthogonal receptor in the population is greater (e.g., at least 3-fold greater, alternatively at least 5-fold greater, alternatively at least 10-fold greater, alternatively at least 50-fold greater, alternatively at least 100-fold greater, or alternatively at least 1000-fold greater) after contacting with the population of cells with the cognate ortholog.

Extracellular Domain: As used herein the term “extracellular domain” or its abbreviation “ECD” refers to the portion of a cell surface protein (e.g., a cell surface receptor) which is outside of the plasma membrane of a cell. The ECD may include the entire extra-cytoplasmic portion of a transmembrane protein, a cell surface or membrane associated protein, a secreted protein, a cell surface targeting protein, or a functional polypeptide fragment thereof comprising the ligand binding domain of the ECD.

Expression Cassette: The term “expression cassette refers” to a recombinant (or synthetic) nucleic acid construct encoding a desired polypeptide operably linked to suitable genetic control elements that are capable of effecting expression of the polypeptide in the host cell to be transformed with the expression vector. The term “operably linked” refers to a linkage of polynucleotide elements in a functional relationship. A nucleic acid sequence is “operably linked” when it is placed into a functional relationship with another nucleic acid sequence. For example, a promoter is operably linked to a coding sequence if it controls the transcription of the polypeptide; a ribosome binding site is operably linked to a coding sequence if it is positioned to permit translation, a nucleic acid encoding signal peptide is operably linked to a nucleic acid sequence encoding such polypeptide if it is expressed as a fusion protein and participates in directing the fusion protein to the cell membrane or in secretion of the polypeptide. Typically, nucleotide sequences that are operably linked are contiguous. However, as enhancers generally function when separated from the promoter by several kilobases and intronic sequences may be of variable lengths, some polynucleotide elements may be operably linked yet physically distant and may even function in trans from a different allele or chromosome.

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

IL2: As used herein, the term “interleukin-2” or “IL2” refers to a naturally occurring IL2 polypeptide that possesses IL2 activity. In some embodiments, IL2 refers to mature wild-type human IL2. Mature wild-type human IL2 (hIL2) occurs as a 133 amino acid polypeptide (less the signal peptide, consisting of an additional 20 N-terminal amino acids), as described in Fujita, et. al., PNAS USA, 80, 7437-7441 (1983). An amino acid sequence of naturally occurring variant of mature wild-type human IL2 (hIL2) is:

(SEQ ID NO: 9)
APTSSSTKKT QLQLEHLLLD LQMILNGINN
YKNPKLTRML TFKFYMPKKA TELKHLQCLE
EELKPLEEVL NLAQSKNFHL RPRDLISNIN
VIVLELKGSE TTFMCEYADE TATIVEFLNR
WITFCQSIIS TLT

As used herein, the numbering of residues is based on the IL2 sequence UniProt ID P60568 excluding the signal peptide as shown in SEQ ID NO:9.

IL2 Activity: The term “IL2 activity” refers to one or more the biological effects on a cell in response to contacting the cell with an effective amount of an IL2 polypeptide. IL2 activity may be measured, for example, in a cell proliferation assay using CTLL-2 mouse cytotoxic T cells, see Gearing, A. J. H. and C. B. Bird (1987) in Lymphokines and Interferons, A Practical Approach. Clemens, M. J. et al. (eds): IRL Press. 295. The specific activity of recombinant human IL2 (rhIL2) is approximately 2.1×10⁴ IU/pg, which is calibrated against recombinant human IL2 WHO International Standard (NIBSC code: 86/500). In some embodiments, for example when the IL2 orthogonal polypeptide of interest exhibits (or is engineered to possess) diminished affinity for CD25, IL2 activity may be assessed in human cells such as YT cells which do not require CD25 to provide signaling through the IL2 receptor but rather are capable of signaling through the intermediate affinity CD122/CD132 receptor. An orthogonal human IL2 of the present disclosure may have less than 20%, alternatively less than about 10%, alternatively less than about 8%, alternatively less than about 6%, alternatively less than about 4%, alternatively less than about 2%, alternatively less than about 1%, alternatively less than about 0.5% of the activity of WHO International Standard (NIBSC code: 86/500) wild-type mature human IL2 when evaluated at similar concentrations in a comparable assay.

IL2 ortholog: As used herein, the term “IL2 ortholog” refers to a variant of IL2 derived from an IL2 parent polypeptide which specifically binds to an orthogonal CD122 ECD and exhibits significantly reduced binding to the extracellular domain of a wild type CD122. In some embodiment the IL2 ortholog exhibits specific binding to a receptor comprising an orthogonal CD122 ECD and (2) the contacting of a cell expressing a membrane spanning receptor comprising the ECD of an orthogonal CD122 polypeptide in an amount sufficient to cause a response results in the a signal characteristic of the signal produced by the intracellular domain (ICD) of said membrane spanning receptor. When the membrane spanning receptor comprises an orthogonal CD122 ECD and CD122 ICD, the binding of an IL2 ortholog to such receptor results in an intracellular signal characteristic of the activation of a Cd25/CD122/CD132 high affinity of CD122/CD132 intermediate affinity IL2 receptor. An IL2 ortholog exhibits significantly reduced binding to wild-type hCD122. The term IL2 orthologs includes IL2 orthogonal variants and modified IL2 orthologs. In some embodiments, the IL2 ortholog is derived from a naturally occurring variant of human IL2 and such human IL2 orthologs may be referred to as “hoCD122” or “hoRb.” Certain modified IL2 polypeptides are provided in Garcia, et al. (United States Patent Application Publication US2018/0228842A1 published Aug. 16, 2018). As used herein, the term IL2 orthologs includes the modified hIL2 polypeptides described in Garcia, et al United State Patent Application Publication US2018/0228842A1 published Aug. 16, 2018. In some embodiments, the affinity of the IL2 ortholog for the extracellular domain of the orthogonal CD122 is comparable to the affinity of wild-type IL2 for ECD of wild-type CD122. In some embodiments, the affinity of the IL2 ortholog for the ECD of the orthogonal CD122 is greater than to the affinity of wild-type IL2 for ECD of wild-type CD122. In some embodiments, the affinity of the IL2 ortholog for the ECD of the orthogonal CD122 is less than to the affinity of wild-type IL2 for the ECD of the wild-type CD122.

Immune Cell: As used herein, the term “immune cell” refers to eukaryotic living cells hematopoietic origin, including primary cells and cell lines derived therefrom, that participate in the in the initiation and/or execution of innate and/or adaptive immune response. In some embodiments, an immune cell refers to an immune cell isolated from a mammalian subject. The term “primary cell(s)” refers to cells taken directly for living tissue and established for growth in vitro that have undergone few population doublings and are often considered more representative of the tissue since they are not transformed.

In some embodiments immune cell that may be isolated from a mammalian subject is a “stem cell.” The term “stem cells” includes but is not limited to adult human stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human stem cells are CD34+ cells.

The scope of immune cells useful in the compositions and methods of the present disclosure include cells of lymphoid lineage including but not limited to B cells, T cells, Natural Killer (NK) cells, NKT cells, cytotoxic T lymphocytes (CTLs), regulatory T cells (Tregs), dendritic cells, killer dendritic cells, and mast cells. In some embodiments immune cell that may be isolated from a mammalian subject is a T cell from the group consisting of inflammatory T-lymphocytes, cytotoxic T-lymphocytes, regulatory T-lymphocytes or helper T-lymphocytes including tumor infiltrating lymphocytes (TILs), CD4+ T-lymphocytes and CD8+ T-lymphocytes, cytotoxic T lymphocytes (CTLs), a regulatory T cell (Tregs), including subsets of CD8+ T lymphocytes of various phenotypes including T effector memory phenotype (Tem), T central memory phenotype (Tcm), terminally differentiated Tcm and Tem cells that express CD45RA (Temra), tissue resident memory (Trm) cells, and peripheral memory (Tpm) cells. CD8+ effector subtypes are characterized in accordance with the following markers as shown in Table 2 below:

TABLE 2
Markers of CD8+ Memory Phenotypes

| Subset | Phenotype |
|---|---|
| Tem | CCR7^{lo}/CD62L^{lo} |
| | Cx3Cr1^{hi}/CD27^{lo} |
| | CD127^{hi} |
| | CD27⁻/CD45RA⁻ (humans) |
| Tcm | CCR7^{hi}/CD62L^{hi} |
| | Cx3Cr1^{lo}/CD27^{hi} |
| | CD127^{hi} |
| | CD27⁺/CD45RA⁻ (humans) |
| Terma (humans) | CCR7⁻/CD27⁻/CD45RA⁺ |
| | CD127^{lo} |
| Trm | CD69^{hi}/CD103^{hi}/CD49a^{hi} |
| | (depending on tissue) |
| | CXCR3^{hi}/KLRG1^{lo}/CCR7^{lo}/ |
| | CD62L^{lo}, CD127^{hi} |
| | Cx3Cr1^{lo/int} |
| Tpm | CCR7^{+/-}/CD62L^{+/-}/CD127^{hi} |
| | Cx3Cr1^{int}/CD27^{hi} |
| Others | CD27^{lo}/CD43^{lo} |
| | KLRG1^{hi}, CD127^{lo} |

### Martin, M. and Badinovac, V., Defining Memory CD8 T Cell (2018) Frontiers in Immunology 9:2692.

In some embodiments, the recombinantly modified mammalian cell is an engineered immune cell, in particular “redirected” immune cells which have been modified to have differential targeting. Examples of such redirected immune cells include T cells recombinantly modified to redirect their specificity to a different target. Examples of such redirected T-cells are to express a chimeric antigen receptor (“CAR-T cells) and T-cell receptor engineered cells (TCR-T cells)

Prior to expansion and genetic modification of the immune cells of the invention, a source of cells can be obtained from a subject through a variety of mammalian sources including peripheral blood mononuclear cells, bone marrow, lymph node tissue, cord blood, thymus tissue, tissue from a site of infection, ascites, pleural effusion, spleen tissue, and tumors. In some embodiments, the immune cell may be obtained from a healthy subject or a from a subject suffering from a disease, disorder or conditions (e.g. cancer of infectious disease). Such immune cell may be party of a mixed population of cells of different phenotypes. Consequently, the isolated cells may be “sorted” by conventional methodologies such as FACS to identify particular subtypes of isolated cells which may be desirable in certain applications and to provide a population of cells enriched for certain subtypes. The cell may also be from a cell line obtained from a transformed T-cell.

Modified cells resistant to an immunosuppressive treatment and susceptible to be obtained by the previous method are encompassed in the scope of the present invention.

In An Amount Sufficient Amount to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is in an vitro test system such as a fluorescent assay. In some embodiments, the test system involves the measurement of a change in the level a parameter of a cell, tissue, or organism reflective of a biological function before and after the application of the test agent to the cell, tissue, or organism. In some embodiments, the indicator (e.g. concentration of phosphorylated STAT5) is reflective of biological function (e.g. activation of the IL2 receptor) of a cell evaluated in a in an assay in response to the administration of a quantity of the test agent (e.g. IL2). In some embodiments, the test system involves the measurement of a change in the level a parameter (e.g. luminescence) of a cell, tissue, or organism (e.g. a mouse injected with luminescent neoplastic cells) reflective of a biological condition (e.g. the presence of a neoplasm) before and after the application of one or more test agents (e.g. a CAR-T cell expressing an orthogonal CD122 in combination with an IL2 ortholog) to the cell, tissue, or organism (e.g. the mouse). In some embodiments, the indicator (e.g. concentration of phosphorylated STAT5) is reflective of biological function (e.g. activation of an IL2 receptor) of a cell (e.g. a T cell) evaluated in a in an assay in response to the administration of a quantity of the test agent (e.g. IL2). “An amount sufficient to effect a response” may be sufficient to be a therapeutically effective amount but “in an amount sufficient to cause a response” may be more or less than a therapeutically effective amount.

In Combination With: As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g. an hoCD122^{pos}/wt hCD122^{neg} cell) is considered to be administered in combination with a second agent (e.g. hoIL2) if the biological effect resulting from the administration of the first agent persists in the subject at the time of administration of the second agent such that the therapeutic effects of the first agent and second agent overlap. For example, the hoCD122^{pos}/wt hCD122^{neg} cell is typically once while the hoIL2 ligand is typically administered more frequently, e.g. daily, BID, or weekly. However, the administration of the first agent (e.g. hoCD122^{pos}/wt hCD122^{neg} cell) provides a therapeutic effect over an extended time and the administration of the second agent (e.g. the hoIL2 ligand) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the hoIL2 ligand and the supplementary agent(s) are administered or applied sequentially, e.g., where one agent is administered prior to one or more other agents. In other embodiments, the hpIL2 mutein and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

Inhibitor: As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Isolated: As used herein the term “isolated” is used in reference to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

Intracellular Domain of the Orthogonal Receptor: As used herein the terms “intracellular domain of the orthogonal receptor” or “ICD-OR” refer to the portion of a transmembrane spanning orthogonal receptor that is inside of the plasma membrane of a cell expressing such transmembrane spanning orthogonal receptor. The ICD-OR may comprise one or more “proliferation signaling domain(s)” or “PSD(s)” which refers to a protein domain which signals the cell to enter mitosis and begin cell growth. Examples include the Janus kinases, including but not limited to, JAK1, JAK2, JAK3, Tyk2, Ptk-2, homologous members of the Janus kinase family from other mammalian or eukaryotic species, the IL2 receptor β and/or γ chains and other subunits from the cytokine receptor superfamily of proteins that may interact with the Janus kinase family of proteins to transduce a signal, or portions, modifications or combinations thereof. Examples of signals include phosphorylation of one or more STAT molecules including but not limited to one or more of STAT1, STAT3, STAT5a, and/or STAT5b.

Kabat Numbering: The term “Kabat numbering” as used herein is recognized in the art and refers to a system of numbering amino acid residues which are more variable than other amino acid residues (e.g., hypervariable) in the heavy and light chain regions of immunoglobulins (Kabat, et al., (1971) Ann. NY Acad. Sci. 190:382-93; Kabat, et al., (1991) Sequences of Proteins of Immunological Interest, Fifth Edition, U.S. Department of Health and Human Services, NIH Publication No. 91-3242). For purposes of the present disclosure, the positioning of CDRs in the variable region of an antibody follows Kabat numbering or simply, “Kabat.”

Ligand: As used herein, the term “ligand” refers to a molecule that specifically binds a receptor and causes a change in the receptor so as to effect a change in the activity of the receptor or a response in cell that expresses that receptor. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

Metastasis: As used herein the term “metastasis” describes the spread of cancer cell from the primary tumor to surrounding tissues and to distant organs.

Modified IL2 ortholog: As used herein the term “modified IL2 orthologs” is used to refer to IL2 orthologs that have been modified by one or more modifications such as pegylation, glycosylation (N- and O-linked), acylation, or polysialylation or by conjugation (either chemical or as fusion proteins) with other polypeptide carrier molecules including but not limited to albumin fusion polypeptides comprising serum albumin (e.g., human serum albumin (HSA) or bovine serum albumin (BSA)), Fc-fusion proteins), targeted IL2 ortholog fusion proteins (such as scFv-IL2 ortholog fusion proteins, VHH-IL2 orthogonal polypeptide fusion proteins) and the like. Modified IL2 orthologs may be prepared to order to enhance one or more properties for example, modulating immunogenicity (conjugation or fusion to immunogens), methods of increasing water solubility, bioavailability, serum half-life, and/or therapeutic half-life; and/or modulating biological activity. Certain modifications can also be useful to, for example, generation of antibodies for use in detection assays (e.g., epitope tags) or to provide for ease of protein purification (e.g. His tags). Modified IL2 orthologs may be prepared to order to enhance one or more properties for example, modulating immunogenicity; methods of increasing water solubility, bioavailability, serum half-life, and/or therapeutic half-life; and/or modulating biological activity. Certain modifications can also be useful to, for example, raise of antibodies for use in detection assays (e.g., epitope tags) and to provide for ease of protein purification. In some embodiments, the modified IL2 ortholog is at least 90%, 91%, 92%, 93%, 94%, 95%, 96%, 97%, 98%, or 99% identical to SEQ ID NO:9 excluding the modifications in the FORMULA #2. Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

Modulate: As used herein, the terms “modulate”, “modulation” and the like refer to the ability of a test agent to cause a response, either positive or negative or directly or indirectly, in a system, including a biological system or biochemical pathway. The term modulator includes both agonists and antagonists.

Myeloid Cell: As used herein, a “myeloid cell” refers to a cell that is derived from a myeloid progenitor cell. Exemplary myeloid cells include but are not limited to granulocytes, monocytes, erythrocytes, and platelets, as well as myeloid progenitor cells that are committed to the myeloid lineage.

Mutein: As used herein, the term “mutein” is used to refer to modified versions of wild type polypeptides comprising modifications to the primary structure (i.e. amino acid sequence) of such polypeptide. The term mutein may refer to the polypeptide itself, a composition comprising the polypeptide, or a nucleic acid sequence that encodes it. In some embodiments, the mutein polypeptide comprises from about one to about ten amino acid modifications relative to the parent polypeptide, alternatively from about one to about five amino acid modifications compared to the parent, alternatively from about one to about three amino acid modifications compared to the parent, alternatively from one to two amino acid modifications compared to the parent, alternatively a single amino acid modification compared to the parent. A mutein may be at least about 99% identical to the parent polypeptide, alternatively at least about 98% identical, alternatively at least about 97% identical, alternatively at least about 95% identical, or alternatively at least about 90% identical.

Neoplastic Disease: As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Numbered in accordance with IL2: The term “numbered in accordance with IL2” as used herein refers to the identification of a location of particular amino acid with reference to the position at which that amino acid normally occurs in the mature sequence of the mature wild type hIL2 (SEQ ID NO: 9). For example, “R81” refers to the eighty-first (numbered from the N-terminus) amino acid, arginine, that occurs in sequence of the mature wild type hIL2.

Numbered in accordance with CD122: The term “numbered in accordance with CD122” as used herein refers to the identification of a location of particular amino acid with reference to the position at which that amino acid normally occurs in the mature sequence of the mature wild type human CD122 (SEQ ID NO. 6). For example, for example H133 refers to the one-hundred thirty third (numbered from the N-terminus) amino acid of the sequence of the mature wild type human CD122.

Operably Linked: The term “operably linked” is used herein to refer to the relationship between molecules, typically polypeptides or nucleic acids, which are arranged in a construct such that each of the functions of the component molecules is retained although the operable linkage may result in the modulation of the activity, either positively or negatively, of the individual components of the construct. For example, the operable linkage of a polyethylene glycol (PEG) molecule to a wild-type protein may result in a construct where the biological activity of the protein is diminished relative to the wild-type molecule, however the two are nevertheless considered operably linked. Alternatively, in the context of a multi-domain receptor comprised of functional domains derived from heterologous sources (e.g., a CAR or OCR), the functional domains of the fusion protein are operably linked when a function characteristic of a first domain of the fusion protein (e.g. ligand binding to the ECD) modulates a function characteristic of a second domain of the fusion protein (e.g., intracellular signaling of the ICD). When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of a particular nucleic acid sequence in a cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Orthogonal Cell: As used herein, the term “orthogonal cell” refers to a mammalian cell which has been recombinantly modified to express an orthogonal CD122 polypeptide. In some embodiments the orthogonal cell a modified human cell (“human orthogonal cell”). The orthogonal cell may be an immune cell, for example a human immune cell. A “human orthogonal immune cell” is human immune cell recombinantly modified to expression a human orthogonal CD122, the immune cell selected from the group consisting of myeloid cells, lymphocytes, peripheral blood mononuclear cells (PBMCs), tumor infiltrating lymphocytes (TILs), T cells, CD8+ T cells, CD25+CD8+ T cells, CAR-T cells, NK cells, CD4+ T cells, and Tregs engineered versions thereof including but limited to engineered TILs, engineered Tregs and engineered NK cells. In some embodiments, the orthogonal cell is CAR-T derived from a human immune cell that has been recombinantly modified to express a human orthogonal CD122 (“hoCAR-T” cell). In some embodiments, the orthogonal cell is a TIL isolated from the neoplasm of a human subject that has been recombinantly modified to express a human orthogonal CD122 (“hoTIL” cell). In some embodiments, the orthogonal cell is NK isolated from a human subject that has been recombinantly modified to express a human orthogonal CD122 (“hoNK” cell). In some embodiments, the cell is a human hematopoietic stem that has been recombinantly modified an orthogonal human CD122 (“hoHSC” cell). As used herein, the term “orthogonal cell” refers to a mammalian cell which has been recombinantly modified to express an orthogonal CD122 polypeptide. The orthogonal cell may incorporate recombinant modifications in addition to the recombinant modifications necessary to express an orthogonal CD122 polypeptide including recombinant modifications including the introduction of nucleic acid molecules encoding marker proteins operably linked to expression control sequences to facilitate expression in the orthogonal cell including but not limited to: marker proteins (proteins conferring antibiotic resistance, fluorescent proteins, or luminescent proteins); biologically active intracellularly proteins including but not limited to DNA or RNA binding proteins, transcription factors including transcriptional repressors or de-repressors, pro-apoptotic proteins, anti-apoptotic proteins and intracellular regulatory proteins; biologically active secreted proteins such as growth factors, peptide hormones, cytokines or chemokines including biologically active therapeutic proteins such as antibodies (the extracellular protein typically comprising a signal peptide or secretion leader sequence to facilitate extracellular transport following expression in the orthogonal cell). Additional recombinant modifications to the orthogonal cells will be apparent to those of skill in the art. In some embodiments, the orthogonal CD122 of the orthogonal cell may comprise an intracellular comprising one or more STAT3 binding motifs.

Orthogonal CD122: As used herein the term “orthogonal CD122” or “CD122 orthogonal receptor” are used interchangeably herein to refer to an CD122 polypeptide variant comprising amino acid substitutions that result in specific binding to an IL2 ortholog that is a cognate ligand for such CD122 polypeptide variant but does not specifically bind to a naturally occurring form of IL2. In one embodiment, the orthogonal CD122 is human CD122 comprising amino acid modifications at as positions 133 and 134 of numbered in accordance with the naturally occurring form of mature human CD122 (SEQ ID NO: 6). In some embodiments, the orthogonal CD122 is a hCD122 molecule comprising the amino acid substitutions H133D and Y134. In one embodiment, the orthogonal receptor is a modified human CD122 wherein the amino acid sequence of the ECD is a 214 amino acid polypeptide of the sequence:

(SEQ ID NO: 1)
AVNGTSQFTC FYNSRANISC VWSQDGALQD
TSCQVHAWPD RRRWNQTCEL LPVSQASWAC
NLILGAPDSQ KLTTVDIVTL RVLCREGVRW
RVMAIQDFKP FENLRLMAPI SLQVVHVETH
RCNISWEISQ ASDFFERHLE FEARTLSPGH
TWEEAPLLTL KQKQEWICLE TLTPDTQYEF
QVRVKPLQGE FTTWSPWSQP LAFRTKPAAL
GKDT

In one embodiment, the orthogonal receptor is a modified human CD122 having the amino acid sequence (less the signal peptide) of the ECD of hoCD122 having substitutions H133D and Y134F and the transmembrane (TM) and intracellular domain (ICD) of the wild-type hCD122 molecule having the amino acid sequence:

(SEQ ID NO: 2)
AVNGTSQFTC FYNSRANISC VWSQDGALQD
TSCQVHAWPD RRRWNQTCEL LPVSQASWAC
NLILGAPDSQ KLTTVDIVTL RVLCREGVRW
RVMAIQDFKP FENLRLMAPI SLQVVHVETH
RCNISWEISQ ASDFFERHLE FEARTLSPGH
TWEEAPLLTL KQKQEWICLE TLTPDTQYEF
QVRVKPLQGE FTTWSPWSQP LAFRTKPAAL
GKDTIPWLGH LLVGLSGAFG FIILVYLLIN
CRNTGPWLKK VLKCNTPDPS KFFSQLSSEH
GGDVQKWLSS PFPSSSFSPG GLAPEISPLE
VLERDKVTQL LLQQDKVPEP ASLSSNHSLT
SCFTNQGYFF FHLPDALEIE ACQVYFTYDP
YSEEDPDEGV AGAPTGSSPQ PLQPLSGEDD
AYCTFPSRDD LLLFSPSLLG GPSPPSTAPG
GSGAGEERMP PSLQERVPRD WDPQPLGPPT
PGVPDLVDFQ PPPELVLREA GEEVPDAGPR
EGVSFPWSRP PGQGEFRALN ARLPLNTDAY
LSLQELQGQD PTHL

“hoCD122” or “hoIL2Rb” are used interchangeably to refers to a variant of hCD122 comprising amino acid substitutions at positions histidine 133 (H133) and tyrosine 134 (Y134) in the ECD of the hCD122 receptor polypeptide.

The orthogonal hCD122 receptor is a variant of hCD122 that comprises one or more amino acid modifications (e.g., deletions or substitutions) at those positions involved in the binding of native cytokine (i.e. wild-type hIL2) to wild-type hCD122 so as to disrupt the binding of the native cytokine (i.e. wt-hIL2) to the orthogonal hCD122. Amino acids involved in the binding of the hIL2 to hCD122 include but are not limited to amino acids R41, R42, Q70, K71, T73, T74, V75, S132, H133, Y134, F135, E136, and/or Q188. In some embodiments, the orthogonal CD122 comprises a one or more substitutions or deletions of amino acids R41, R42, Q70, K71, T73, T74, V75, S132, H133, Y134, F135, E136, and/or Q188. In some embodiments, the orthogonal CD122 comprises one or more substitutions or deletions of the amino acids Q70, T73, H133, and/or Y134. In some embodiments, the orthogonal CD122 comprises one or more substitutions or deletions of the amino acids H133 and/or Y134. In some embodiments, the orthogonal CD122 comprises one or more substitutions or deletions of the amino acids H133 and/or Y134. In some embodiments, the orthogonal CD122 comprises one or more substitutions or deletions of the amino acids H133 and Y134.

In some embodiments CD122 ortho comprises the amino acid substitutions at position 133 from histidine to aspartic acid (H133D), glutamic acid (H133E) or lysine (H133K) and/or amino acid substitutions at position 134 to from tyrosine to phenylalanine (Y134F), glutamic acid (Y134E), or arginine (Y134R). In one embodiment, the orthogonal CD122 ortho is a hCD122 molecule having amino acid substitutions H133D and Y134F. In one embodiment, the CD122 ortho is a polypeptide having the amino acid sequence of SEQ ID NO: 2.aA representative nucleic acid sequence encoding human orthogonal CD122 (hoCD122) of SEQ ID NO:27 is provided below:

(SEQ ID NO: 27)
ATGGCGGCCCCTGCTCTGTCCTGGCGTCTGCCCCTC
CTCATCCTCCTCCTGCCCCTGGCTACCTCTTGGGC
ATCTGCAGCGGTGAATGGCACTTCCCAGTTCACAT
GCTTCTACAACTCGAGAGCCAACATCTCCTGTGTC
TGGAGCCAAGATGGGGCTCTGCAGGACACTTCCTG
CCAAGTCCATGCCTGGCCGGACAGACGGCGGTGGA
ACCAAACCTGTGAGCTGCTCCCCGTGAGTCAAGCA
TCCTGGGCCTGCAACCTGATCCTCGGAGCCCCAGA
TTCTCAGAAACTGACCACAGTTGACATCGTCACCC
TGAGGGTGCTGTGCCGTGAGGGGGTGCGATGGAGG
GTGATGGCCATCCAGGACTTCAAGCCCTTTGAGAA
CCTTCGCCTGATGGCCCCCATCTCCCTCCAAGTTG
TCCACGTGGAGACCCACAGATGCAACATAAGCTGG
GAAATCTCCCAAGCCTCCgACTtCTTTGAAAGACA
CCTGGAGTTCGAGGCCCGGACGCTGTCCCCAGGCC
ACACCTGGGAGGAGGCCCCCCTGCTGACTCTCAAG
CAGAAGCAGGAATGGATCTGCCTGGAGACGCTCAC
CCCAGACACCCAGTATGAGTTTCAGGTGCGGGTCA
AGCCTCTGCAAGGCGAGTTCACGACCTGGAGCCCC
TGGAGCCAGCCCCTGGCCTTCAGGACAAAGCCTGC
AGCCCTTGGGAAGGACACCATTCCGTGGCTCGGCC
ACCTCCTCGTGGGTCTCAGCGGGGCTTTTGGCTTC
ATCATCTTAGTGTACTTGCTGATCAACTGCAGGAA
CACCGGGCCATGGCTGAAGAAGGTCCTGAAGTGTA
ACACCCCAGACCCCTCGAAGTTCTTTTCCCAGCTG
AGCTCAGAGCATGGAGGAGACGTCCAGAAGTGGCT
CTCTTCGCCCTTCCCCTCATCGTCCTTCAGCCCTG
GCGGCCTGGCACCTGAGATCTCGCCACTAGAAGTG
CTGGAGAGGGACAAGGTGACGCAGCTGCTCCTGCA
GCAGGACAAGGTGCCTGAGCCCGCATCCTTAAGCA
GCAACCACTCGCTGACCAGCTGCTTCACCAACCAG
GGTTACTTCTTCTTCCACCTCCCGGATGCCTTGGA
GATAGAGGCCTGCCAGGTGTACTTTACTTACGACC
CCTACTCAGAGGAAGACCCTGATGAGGGTGTGGCC
GGGGCACCCACAGGGTCTTCCCCCCAACCCCTGCA
GCCTCTGTCAGGGGAGGACGACGCCTACTGCACCT
TCCCCTCCAGGGATGACCTGCTGCTCTTCTCCCCC
AGTCTCCTCGGTGGCCCCAGCCCCCCAAGCACTGC
CCCTGGGGGCAGTGGGGCCGGTGAAGAGAGGATGC
CCCCTTCTTTGCAAGAAAGAGTCCCCAGAGACTGG
GACCCCCAGCCCCTGGGGCCTCCCACCCCAGGAGT
CCCAGACCTGGTGGATTTTCAGCCACCCCCTGAGC
TGGTGCTGCGAGAGGCTGGGGAGGAGGTCCCTGAC
GCTGGCCCCAGGGAGGGAGTCAGTTTCCCCTGGTC
CAGGCCTCCTGGGCAGGGGGAGTTCAGGGCCCTTA
ATGCTCGCCTGCCCCTGAACACTGATGCCTACTTG
TCCCTCCAAGAACTCCAGGGTCAGGACCCAACTCA
CTTGGTGTAG.
G.

Orthogonal Chimeric Receptor: As used herein, the terms “orthogonal chimeric receptor” or “OCR” are used interchangeably to refer a polypeptide the extracellular domain (ECD) of which is derived from an hoCD122 or functional subfragments thereof, operably linked to an intracellular domain (ICD) of a heterologous receptor subunit including but not limited to the ICD of from the IL-4 receptor alpha subunit (IL-4Rα), the IL-7 receptor alpha subunit (IL-7Rα), the IL-9 receptor alpha subunit (IL-9Rα), the IL-15R receptor alpha subunit (IL-15Rα), IL-21 receptor (IL-21R) or the erythropoietin receptor (EpoR), or a functional fragment thereof. The ECD and ICD of the OCR may be operably linked via a polypeptide sequence comprising the transmembrane domain of the receptor from which the ICD or ECD of the OCR are derived. In one embodiment, ICD or ECD of the OCR are operably linked via a polypeptide comprising the transmembrane domain of the receptor from which the ECD is derived. In one embodiment, ICD or ECD of the OCR are operably linked via a polypeptide comprising the transmembrane domain of the receptor from which the ICD is derived. Examples of OCRs are described in Garcia, et al., International Patent Application No. PCT/US2020/050232 published Mar. 18, 2021 as WO 2021/050752 and exemplified below.

An OCR comprising a hoCD122 ECD and IL7ICD (hoCD122-IL7R) protein sequence:

(SEQ ID NO: 19)
MAAPALSWRLPLLILLLPLATSWASAAVNGTSQFTC
FYNSRANISCVWSQDGALQDTSCQVHAWPDRRRWN
QTCELLPVSQASWACNLILGAPDSQKLTTVDIVTL
RVLCREGVRWRVMAIQDFKPFENLRLMAPISLQVV
HVETHRCNISWEISQASDFFERHLEFEARTLSPGH
TWEEAPLLTLKQKQEWICLETLTPDTQYEFQVRVK
PLQGEFTTWSPWSQPLAFRTKPANNSSGEMDPILL
TISILSFFSVALLVILACVLWKKRIKPIVWPSLPD
HKKTLEHLCKKPRKNLNVSFNPESFLDCQIHRVDD
IQARDEVEGFLQDTFPQQLEESEKQRLGGDVQSPN
CPSEDVVITPESFGRDSSLTCLAGNVSACDAPILS
SSRSLDCRESGKNGPHVYQDLLLSLGTTNSTLPPP
FSLQSGILTLNPVAQGQPILTSLGSNQEEAYVTMS
SFYQNQ

wherein residues 1-234 are derived from hoCD122 and residues 235-462 are derived from the ICD of the human IL-7Rα receptor (underlined) and encoded by the nucleic acid sequence

(SEQ ID NO: 20)
ATGGCGGCCCCTGCTCTGTCCTGGCGTCTGCCCCT
CCTCATCCTCCTCCTGCCCCTGGCTACCTCTTGGG
CATCTGCAGCGGTGAATGGCACTTCCCAGTTCACA
TGCTTCTACAACTCGAGAGCCAACATCTCCTGTGT
CTGGAGCCAAGATGGGGCTCTGCAGGACACTTCCT
GCCAAGTCCATGCCTGGCCGGACAGACGGCGGTGG
AACCAAACCTGTGAGCTGCTCCCCGTGAGTCAAGC
ATCCTGGGCCTGCAACCTGATCCTCGGAGCCCCAG
ATTCTCAGAAACTGACCACAGTTGACATCGTCACC
CTGAGGGTGCTGTGCCGTGAGGGGGTGCGATGGAG
GGTGATGGCCATCCAGGACTTCAAGCCCTTTGAGA
ACCTTCGCCTGATGGCCCCCATCTCCCTCCAAGTT
GTCCACGTGGAGACCCACAGATGCAACATAAGCTG
GGAAATCTCCCAAGCCTCCgACTtCTTTGAAAGAC
ACCTGGAGTTCGAGGCCCGGACGCTGTCCCCAGGC
CACACCTGGGAGGAGGCCCCCCTGCTGACTCTCAA
GCAGAAGCAGGAATGGATCTGCCTGGAGACGCTCA
CCCCAGACACCCAGTATGAGTTTCAGGTGCGGGTC
AAGCCTCTGCAAGGCGAGTTCACGACCTGGAGCCC
CTGGAGCCAGCCCCTGGCCTTCAGGACAAAGCCTG
CAAATAATAGCTCAGGGGAGATGGATCCTATCTTA
CTAACCATCAGCATTTTGAGTTTTTTCTCTGTCGC
TCTGTTGGTCATCTTGGCCTGTGTGTTATGGAAAA
AAAGGATTAAGCCTATCGTATGGCCCAGTCTCCCC
GATCATAAGAAGACTCTGGAACATCTTTGTAAGAA
ACCAAGAAAAAATTTAAATGTGAGTTTCAATCCTG
AAAGTTTCCTGGACTGCCAGATTCATAGGGTGGAT
GACATTCAAGCTAGAGATGAAGTGGAAGGTTTTCT
GCAAGATACGTTTCCTCAGCAACTAGAAGAATCTG
AGAAGCAGAGGCTTGGAGGGGATGTGCAGAGCCCC
AACTGCCCATCTGAGGATGTAGTCATCACTCCAGA
AAGCTTTGGAAGAGATTCATCCCTCACATGCCTGG
CTGGGAATGTCAGTGCATGTGACGCCCCTATTCTC
TCCTCTTCCAGGTCCCTAGACTGCAGGGAGAGTGG
CAAGAATGGGCCTCATGTGTACCAGGACCTCCTGC
TTAGCCTTGGGACTACAAACAGCACGCTGCCCCCT
CCATTTTCTCTCCAATCTGGAATCCTGACATTGAA
CCCAGTTGCTCAGGGTCAGCCCATTCTTACTTCCC
TGGGATCAAATCAAGAAGAAGCATATGTCACCATG
TCCAGCTTCTACCAAAACCAGTGA

An OCR comprising a hoCD122 ECD and an IL9Ra ICD (hoCD122-IL9R) coding sequence:

(SEQ ID NO: 21)
MAAPALSWRLPLLILLLPLATSWASAAVNGTSQFT
CFYNSRANISCVWSQDGALQDTSCQVHAWPDRRRW
NQTCELLPVSQASWACNLILGAPDSQKLTTVDIVT
LRVLCREGVRWRVMAIQDFKPFENLRLMAPISLQV
VHVETHRCNISWEISQASDFFERHLEFEARTLSPG
HTWEEAPLLTLKQKQEWICLETLTPDTQYEFQVRV
KPLQGEFTTWSPWSQPLAFRTKPAQRQGPLIPPWG
WPGNTLVAVSIFLLLTGPTYLLFKLSPRVKRIFYQ
NVPSPAMFFQPLYSVHNGNFQTWMGAHGAGVLLSQ
DCAGTPQGALEPCVQEATALLTCGPARPWKSVALE
EEQEGPGTRLPGNLSSEDVLPAGCTEWRVQTLAYL
PQEDWAPTSLTRPAPPDSEGSRSSSSSSSSNNNNY
CALGCYGGWHLSALPGNTQSSGPIPALACGLSCDH
QGLETQQGVAWVLAGHCQRPGLHEDLQGMLLPSVL
SKARSWTF

wherein residues 1-234 are derived from hoCD122 and residues 235-498 are derived from human IL-9R (underlined)

(SEQ ID NO: 22)
ATGGCGGCCCCTGCTCTGTCCTGGCGTCTGCCCCT
CCTCATCCTCCTCCTGCCCCTGGCTACCTCTTGGG
CATCTGCAGCGGTGAATGGCACTTCCCAGTTCACA
TGCTTCTACAACTCGAGAGCCAACATCTCCTGTGT
CTGGAGCCAAGATGGGGCTCTGCAGGACACTTCCT
GCCAAGTCCATGCCTGGCCGGACAGACGGCGGTGG
AACCAAACCTGTGAGCTGCTCCCCGTGAGTCAAGC
ATCCTGGGCCTGCAACCTGATCCTCGGAGCCCCAG
ATTCTCAGAAACTGACCACAGTTGACATCGTCACC
CTGAGGGTGCTGTGCCGTGAGGGGGTGCGATGGAG
GGTGATGGCCATCCAGGACTTCAAGCCCTTTGAGA
ACCTTCGCCTGATGGCCCCCATCTCCCTCCAAGTT
GTCCACGTGGAGACCCACAGATGCAACATAAGCTG
GGAAATCTCCCAAGCCTCCgACTtCTTTGAAAGAC
ACCTGGAGTTCGAGGCCCGGACGCTGTCCCCAGGC
CACACCTGGGAGGAGGCCCCCCTGCTGACTCTCAA
GCAGAAGCAGGAATGGATCTGCCTGGAGACGCTCA
CCCCAGACACCCAGTATGAGTTTCAGGTGCGGGTC
AAGCCTCTGCAAGGCGAGTTCACGACCTGGAGCCC
CTGGAGCCAGCCCCTGGCCTTCAGGACAAAGCCTG
CACAGAGACAAGGCCCTCTGATCCCACCCTGGGGG
TGGCCAGGCAACACCCTTGTTGCTGTGTCCATCTT
TCTCCTGCTGACTGGCCCGACCTACCTCCTGTTCA
AGCTGTCGCCCAGGGTGAAGAGAATCTTCTACCAG
AACGTGCCCTCTCCAGCGATGTTCTTCCAGCCCCT
CTACAGTGTACACAATGGGAACTTCCAGACTTGGA
TGGGGGCCCACGGGGCCGGTGTGCTGTTGAGCCAG
GACTGTGCTGGCACCCCACAGGGAGCCTTGGAGCC
CTGCGTCCAGGAGGCCACTGCACTGCTCACTTGTG
GCCCAGCGCGTCCTTGGAAATCTGTGGCCCTGGAG
GAGGAACAGGAGGGCCCTGGGACCAGGCTCCCGGG
GAACCTGAGCTCAGAGGATGTGCTGCCAGCAGGGT
GTACGGAGTGGAGGGTACAGACGCTTGCCTATCTG
CCACAGGAGGACTGGGCCCCCACGTCCCTGACTAG
GCCGGCTCCCCCAGACTCAGAGGGCAGCAGGAGCA
GCAGCAGCAGCAGCAGCAGCAACAACAACAACTAC
TGTGCCTTGGGCTGCTATGGGGGATGGCACCTCTC
AGCCCTCCCAGGAAACACACAGAGCTCTGGGCCCA
TCCCAGCCCTGGCCTGTGGCCTTTCTTGTGACCAT
CAGGGCCTGGAGACCCAGCAAGGAGTTGCCTGGGT
GCTGGCTGGTCACTGCCAGAGGCCTGGGCTGCATG
AGGACCTCCAGGGCATGTTGCTCCCTTCTGTCCTC
AGCAAGGCTCGGTCCTGGACATTCTA

An OCR comprising a hoCD122 ECD and an IL21Ra ICD (hoCD122-IL21R) coding sequence:

(SEQ ID NO: 23)
MAAPALSWRLPLLILLLPLATSWASAAVNGTSQFT
CFYNSRANISCVWSQDGALQDTSCQVHAWPDRRRW
NQTCELLPVSQASWACNLILGAPDSQKLTTVDIVT
LRVLCREGVRWRVMAIQDFKPFENLRLMAPISLQV
VHVETHRCNISWEISQASDFFERHLEFEARTLSPG
HTWEEAPLLTLKQKQEWICLETLTPDTQYEFQVRV
KPLQGEFTTWSPWSQPLAFRTKPAEELKEGWNPHL
LLLLLLVIVFIPAFWSLKTHPLWRLWKKIWAVPSP
ERFFMPLYKGCSGDFKKWVGAPFTGSSLELGPWSP
EVPSTLEVYSCHPPRSPAKRLQLTELQEPAELVES
DGVPKPSFWPTAQNSGGSAYSEERDRPYGLVSIDT
VTVLDAEGPCTWPCSCEDDGYPALDLDAGLEPSPG
LEDPLLDAGTTVLSCGCVSAGSPGLGGPLGSLLDR
LKPPLADGEDWAGGLPWGGRSPGGVSESEAGSPLA
GLDMDTFDSGFVGSDCSSPVECDFTSPGDEGPPRS
YLRQWVVIPPPLSSPGPQAS

wherein residues 1-234 are derived from hoCD122 and residues 235-545 human IL-21R (underlined) and which is encoded by the polynucleotide of the sequence

(SEQ ID NO: 24)
ATGGCGGCCCCTGCTCTGTCCTGGCGTCTGCCCCT
CCTCATCCTCCTCCTGCCCCTGGCTACCTCTTGGG
CATCTGCAGCGGTGAATGGCACTTCCCAGTTCACA
TGCTTCTACAACTCGAGAGCCAACATCTCCTGTGT
CTGGAGCCAAGATGGGGCTCTGCAGGACACTTCCT
GCCAAGTCCATGCCTGGCCGGACAGACGGCGGTGG
AACCAAACCTGTGAGCTGCTCCCCGTGAGTCAAGC
ATCCTGGGCCTGCAACCTGATCCTCGGAGCCCCAG
ATTCTCAGAAACTGACCACAGTTGACATCGTCACC
CTGAGGGTGCTGTGCCGTGAGGGGGTGCGATGGAG
GGTGATGGCCATCCAGGACTTCAAGCCCTTTGAGA
ACCTTCGCCTGATGGCCCCCATCTCCCTCCAAGTT
GTCCACGTGGAGACCCACAGATGCAACATAAGCTG
GGAAATCTCCCAAGCCTCCgACTtCTTTGAAAGAC
ACCTGGAGTTCGAGGCCCGGACGCTGTCCCCAGGC
CACACCTGGGAGGAGGCCCCCCTGCTGACTCTCAA
GCAGAAGCAGGAATGGATCTGCCTGGAGACGCTCA
CCCCAGACACCCAGTATGAGTTTCAGGTGCGGGTC
AAGCCTCTGCAAGGCGAGTTCACGACCTGGAGCCC
CTGGAGCCAGCCCCTGGCCTTCAGGACAAAGCCTG
CAGAGGAGTTAAAGGAAGGCTGGAACCCTCACCTG
CTGCTTCTCCTCCTGCTTGTCATAGTCTTCATTCC
TGCCTTCTGGAGCCTGAAGACCCATCCATTGTGGA
GGCTATGGAAGAAGATATGGGCCGTCCCCAGCCCT
GAGCGGTTCTTCATGCCCCTGTACAAGGGCTGCAG
CGGAGACTTCAAGAAATGGGTGGGTGCACCCTTCA
CTGGCTCCAGCCTGGAGCTGGGACCCTGGAGCCCA
GAGGTGCCCTCCACCCTGGAGGTGTACAGCTGCCA
CCCACCACGGAGCCCGGCCAAGAGGCTGCAGCTCA
CGGAGCTACAAGAACCAGCAGAGCTGGTGGAGTCT
GACGGTGTGCCCAAGCCCAGCTTCTGGCCGACAGC
CCAGAACTCGGGGGGCTCAGCTTACAGTGAGGAGA
GGGATCGGCCATACGGCCTGGTGTCCATTGACACA
GTGACTGTGCTAGATGCAGAGGGGCCATGCACCTG
GCCCTGCAGCTGTGAGGATGACGGCTACCCAGCCC
TGGACCTGGATGCTGGCCTGGAGCCCAGCCCAGGC
CTAGAGGACCCACTCTTGGATGCAGGGACCACAGT
CCTGTCCTGTGGCTGTGTCTCAGCTGGCAGCCCTG
GGCTAGGAGGGCCCCTGGGAAGCCTCCTGGACAGA
CTAAAGCCACCCCTTGCAGATGGGGAGGACTGGGC
TGGGGGACTGCCCTGGGGTGGCCGGTCACCTGGAG
GGGTCTCAGAGAGTGAGGCGGGCTCACCCCTGGCC
GGCCTGGATATGGACACGTTTGACAGTGGCTTTGT
GGGCTCTGACTGCAGCAGCCCTGTGGAGTGTGACT
TCACCAGCCCCGGGGACGAAGGACCCCCCCGGAGC
TACCTCCGCCAGTGGGTGGTCATTCCTCCGCCACT
TTCGAGCCCTGGACCCCAGGCCAGCTAA

An OCR comprising a hoCD122 ECD and an ICD derived from the Epo having the amino acid sequence:

(SEQ ID NO: 25)
MAAPALSWRLPLLILLLPLATSWASAAVNGTSQFT
CFYNSRANISCVWSQDGALQDTSCQVHAWPDRRRW
NQTCELLPVSQASWACNLILGAPDSQKLTTVDIVT
LRVLCREGVRWRVMAIQDFKPFENLRLMAPISLQV
VHVETHRCNISWEISQASDFFERHLEFEARTLSPG
HTWEEAPLLTLKQKQEWICLETLTPDTQYEFQVRV
KPLQGEFTTWSPWSQPLAFRTKPASDLDPLILTLS
LILWILVLLTVLALLSHRRALKQKIWPGIPSPESE
FEGLFTTHKGNFQLWLYQNDGCLWWSPCTPFTEDP
PASLEVLSERCWGTMQAVEPGTDDEGPLLEPVGSE
HAQDTYLVLDKWLLPRNPPSEDLPGPGGSVDIVAM
DEGSEASSCSSALASKPSPEGASAASFEYTILDPS
SQLLRPWTLCPELPPTPPHLKYLYLWSDSGISTDY
SSGDSQGAQGGLSDGPYSNPYENSLIPAAEPLPPS
YVACS

wherein residues 1-234 are derived from hoCD122 and residues 235-497 are derived from human EpoR (underlined) and can be encoded by the polynucleotide of the sequence:

(SEQ ID NO: 26)
ATGGCGGCCCCTGCTCTGTCCTGGCGTCTGCCCCTC
CTCATCCTCCTCCTGCCCCTGGCTACCTCTTGGGCA
TCTGCAGCGGTGAATGGCACTTCCCAGTTCACATGC
TTCTACAACTCGAGAGCCAACATCTCCTGTGTCTGG
AGCCAAGATGGGGCTCTGCAGGACACTTCCTGCCAA
GTCCATGCCTGGCCGGACAGACGGCGGTGGAACCAA
ACCTGTGAGCTGCTCCCCGTGAGTCAAGCATCCTGG
GCCTGCAACCTGATCCTCGGAGCCCCAGATTCTCAG
AAACTGACCACAGTTGACATCGTCACCCTGAGGGTG
CTGTGCCGTGAGGGGGTGCGATGGAGGGTGATGGCC
ATCCAGGACTTCAAGCCCTTTGAGAACCTTCGCCTG
ATGGCCCCCATCTCCCTCCAAGTTGTCCACGTGGAG
ACCCACAGATGCAACATAAGCTGGGAAATCTCCCAA
GCCTCCgACTtCTTTGAAAGACACCTGGAGTTCGAG
GCCCGGACGCTGTCCCCAGGCCACACCTGGGAGGAG
GCCCCCCTGCTGACTCTCAAGCAGAAGCAGGAATGG
ATCTGCCTGGAGACGCTCACCCCAGACACCCAGTAT
GAGTTTCAGGTGCGGGTCAAGCCTCTGCAAGGCGAG
TTCACGACCTGGAGCCCCTGGAGCCAGCCCCTGGCC
TTCAGGACAAAGCCTGCAAGCGACCTGGACCCCCTC
ATCCTGACGCTCTCCCTCATCCTCGTGGTCATCCTG
GTGCTGCTGACCGTGCTCGCGCTGCTCTCCCACCGC
CGGGCTCTGAAGCAGAAGATCTGGCCTGGCATCCCG
AGCCCAGAGAGCGAGTTTGAAGGCCTCTTCACCACC
CACAAGGGTAACTTCCAGCTGTGGCTGTACCAGAAT
GATGGCTGCCTGTGGTGGAGCCCCTGCACCCCCTTC
ACGGAGGACCCACCTGCTTCCCTGGAAGTCCTCTCA
GAGCGCTGCTGGGGGACGATGCAGGCAGTGGAGCCG
GGGACAGATGATGAGGGCCCCCTGCTGGAGCCAGTG
GGCAGTGAGCATGCCCAGGATACCTATCTGGTGCTG
GACAAATGGTTGCTGCCCCGGAACCCGCCCAGTGAG
GACCTCCCAGGGCCTGGTGGCAGTGTGGACATAGTG
GCCATGGATGAAGGCTCAGAAGCATCCTCCTGCTCA
TCTGCTTTGGCCTCGAAGCCCAGCCCAGAGGGAGCC
TCTGCTGCCAGCTTTGAGTACACTATCCTGGACCCC
AGCTCCCAGCTCTTGCGTCCATGGACACTGTGCCCT
GAGCTGCCCCCTACCCCACCCCACCTAAAGTACCTG
TACCTTGTGGTATCTGACTCTGGCATCTCAACTGAC
TACAGCTCAGGGGACTCCCAGGGAGCCCAAGGGGGC
TTATCCGATGGCCCCTACTCCAACCCTTATGAGAAC
AGCCTTATCCCAGCCGCTGAGCCTCTGCCCCCCAGC
TATGTGGCTTGCTCTTAG.

Orthogonal Human IL2: The term “orthogonal hIL2” or “hoIL2” refers to a variant of hIL2 that selectively and specifically binds to the ECD of an orthogonal hCD122 receptor or OCR and result in intracellular signaling. Naturally-occurring human CD25 nucleic acid and protein sequences may be found as Genbank accession numbers NM__000417 and NP_0004Q8 respectively. Examples of hoIL2 molecules are provided in FORMULA #2.

STK-008: An exemplary hoIL2 of Formula #2 is the human IL2 mutein comprising the amino acid substitutions L18R, Q22E and Q126H and additionally comprising a deletion of Ala1 referred to herein as des-Ala1 REH, REH and STK-008. The amino acid sequence of STK-008 is provided below (SEQ ID NO:31):

(SEQ ID NO: 31)
PTSSSTKKTQLQLEHLRLDLEMILNGINNYKNPKLT
RMLTFKFYMPKKATELKHLQCLEEELKPLEEVLNLA
QSKNFHLRPRDLISNINVIVLELKGSETTFMCEYAD
ETATIVEFLNRWITFCHSIISTLT

STK-011: A second exemplary hoIL2 of Formula #2 is the human IL2 mutein comprising the amino acid substitutions L18R, Q22E and Q126K and additionally comprising a deletion of Ala1 referred to herein as des-Ala1 REK, REK and STK-011. The amino acid sequence of STK-011 is provided below (SEQ ID NO:32):

(SEQ ID NO: 32)
PTSSSTKKTQLQLEHLRLDLEMILNGINNYKNPKLT
RMLTFKFYMPKKATELKHLQCLEEELKPLEEVLNLA
QSKNFHLRPRDLISNINVIVLELKGSETTFMCEYAD
ETATIVEFLNRWITFCKSIISTLT

Orthogonal Receptor: As used herein the term “orthogonal receptor” refers to a variant of receptor, the orthogonal receptor comprising modifications to the amino acid sequence so that the orthogonal receptor exhibits significantly reduced binding to its cognate ligand but exhibits specific binding for an orthogonal ligand engineered to interact with the orthogonal receptor. In some embodiments, the orthogonal receptor may comprise an extracellular domain that is exhibits significantly reduced binding to its cognate native ligand, while an orthogonal ligand exhibits significantly reduced binding to the ECD of its cognate native receptor(s). The term “orthogonal receptors” includes orthogonal chimeric receptors. In some embodiments, the affinity of the orthogonal ligand for the cognate orthogonal receptor exhibits affinity comparable to the affinity of the native ligand for the native receptor, e.g. having an affinity that is least about 1% of the native cytokine receptor pair affinity, at least about 5%, at least about 10%, at least about 25%, at least about 50%, at least about 75%, at least about 100%, and may be higher, e.g. 2×, 3×, 4×, 5×, 10× or more of the affinity of the native cytokine for the native receptor. An orthogonal receptor may be referred to by the parent molecule from which it was derived (e.g. orthogonal CD122) or by the cognate ligand from which the orthogonal ligand for the orthogonal receptor was derived (e.g. orthogonal IL2 receptor).

Ortholog: As used herein the term “ortholog” refers to a ligand component of an orthogonal ligand/receptor pair and refers to a polypeptide incorporating modifications to its primary structure to provide polypeptide variant that exhibits: (a) significantly reduced affinity to its native cognate receptor (i.e., the native receptor for the parent polypeptide from which the ortholog is derived); and (b) specific binding a engineered orthogonal receptor which is a variant of the cognate receptor for the ortholog. Upon binding of the ortholog to the orthogonal receptor (which is expressed on surface of cell which has been modified by recombinant DNA technology to incorporate a nucleic acid sequence encoding the orthogonal receptor operably linked to control elements to effect the expression of the orthogonal receptor in the recombinantly modified cell), the activated orthogonal receptor initiates signaling that is transduced through native cellular elements to provide for a biological activity that mimics that native response of the cognate but which is specific to the recombinantly modified cell population expressing the orthogonal receptor. In some embodiments of the invention, orthologs possess significant selectivity for the orthogonal receptor relative to the cognate receptor and optionally possessing significantly reduced potency with respect to the cognate receptor. Selectivity is typically assessed by activity measured in an assay characteristic of the activity induced in response to ligand/receptor binding. In some embodiments, the ortholog possesses at least 5 fold, alternatively at least 10 fold, alternatively at least 20 fold, alternatively at least 30 fold, alternatively at least 40 fold, alternatively at least 50 fold, alternatively at least 100 fold, alternatively at least 200 fold difference in EC50 as measured in the same assay.

Parent Polypeptide: As used herein the terms “parent polypeptide”, “parent protein”, “precursor polypeptide”, or “precursor protein” are used interchangeably to refer to unmodified polypeptide that is subsequently modified to generate a variant polypeptide. A parent polypeptide may be a wild-type (or native) polypeptide. Parent polypeptide may refer to the polypeptide itself or compositions that comprise the parent polypeptide.

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. An IL2 partial agonist of the present disclosure may have greater than 10%, alternatively greater than 20%, alternatively greater than 30%, alternatively greater than 40%, alternatively greater than 50%, alternatively greater than 60%, or alternatively greater than 70% of the activity of WHO International Standard (NIBSC code: 86/500) wild type mature human IL2 when evaluated at similar concentrations in a comparable assay.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Receptor: As used herein, the term “receptor” refers to a polypeptide having a domain that specifically binds a ligand that binding of the ligand results in a change to at least one biological property of the polypeptide. I some embodiments, the receptor is a “soluble” receptor that is not associated with a cell surface. The soluble form of hCD25 is an example of a soluble receptor that specifically binds hIL2. In some embodiments, the receptor is a cell surface receptor that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a heteromultimeric including heterodimeric (e.g. the intermediate affinity CD122/CD132 IL2 receptor), heterotrimeric (e.g. the high affinity CD25/CD122/CD132 hIL2 receptor) or homomultimeric (homodimeric, homotrimeric, homotetrameric) complex that results in the activation of an intracellular signaling cascade (e.g. the Jak/STAT pathway).

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and is frequently abbreviated with a lower case “r” (e.g. rhIL2) to denote the method by which the protein was produced. Similarly a cell is referred to as a “recombinant cell” if the cell has been modified by the incorporation (e.g. transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. As used herein, the term “engineered” when used with respect to cell is used to refer the cell when modified using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. Examples of such standard protocols to assess proliferation of CD3 activated primary human T-cells include bioluminescent assay that generates a luminescent signal that is proportional to the amount of ATP present which is directly proportional to the number of cells present in culture as described in Crouch, et al. (1993) “The use of ATP bioluminescence as a measure of cell proliferation and cytotoxicity” J. Immunol. Methods 160: 81-8 or a standardized commercially available assay system such as the CellTiter-Glo® 2.0 Cell Viability Assay or CellTiter-Glo® 3D Cell Viability kits commercially available from Promega Corporation, 2800 Woods Hollow Road, Madison Wis. 53711 as catalog numbers G9241 and G9681 respectively in substantial accordance with the instructions provided by the manufacturer. In some embodiments, the level of activation of T-cells in response to the administration of a test agent may be determined by flow cytometric methods as described as determined by the level of STAT5 phosphorylation in accordance with methods well known in the art. STAT5 phosphorylation may be measured using flow cytometric techniques as described in Horta, et al. supra., Garcia, et al., supra, or commercially available kits such as the Phospho-STAT5 (Tyr694) kit (commercially available from Perkin-Elmer/cisbio Waltham Mass. as Part Number 64AT5PEG) in substantial accordance with the teaching of the manufacturer. When the abbreviation EC^{ACT} used with a subscript this is provided to indicate the concentration of the test agent sufficient to produce the indicated percentage of maximal STAT5 phosphorylation in a T cell in response to the application of the test agent as measured in accordance with the test protocol. By way of illustration, the abbreviation EC₃₀^{PRO} may be used with respect to a hIL2 ortholog to indicate the concentration associated with 30% of a maximal level of STAT5 phosphorylation in a T cell in in response with respect to such hIL2 ortholog as measured with the Phospho-STAT5 (Tyr694) kit.

In some instances, there are standardized accepted measures of biological activity that have been established for a molecule. For example with respect to hIL2 potency, the standard methodology for the evaluation of hIL2 potency in international units (IU) is measured in the murine cytotoxic T cell line CTLL-2 in accordance with standardized procedures as more fully described in Wadhwa, et al. (2013) “The 2nd International standard for Interleukin-2 (IL2) Report of a collaborative study” Journal of Immunological Methods 397:1-7. It should be noted in the context of the present disclosure that the murine IL2 receptor functions differently than the human IL2 receptor, particularly with respect to need for all components of the trimeric receptor complex to provide intracellular signal transduction signaling (e.g. STAT5 phosphorylation). See, e.g. Horta, et al., (2019) “Human and murine IL2 receptors differentially respond to the human-IL2 component of immunocytokines” Oncoimmunology 8(6):e1238538-1, e1238538-15 and Nemoto, et al. (1995) “Differences in the interleukin-2 (IL2) receptor system in human and mouse: alpha chain is require for formation of the functional mouse IL2 receptor” European J Immunology 25(11)3001-5. Consequently, when evaluating the activity of a hIL2 variant, particularly with respect to affinity for CD25 or activation of cells with respect to CD25 status the use of human cells or systems that recapitulate the biology of the human low, intermediate and high affinity IL2 receptors and receptor complexes is preferred and a molecule that exhibits selective binding or activation in a murine test system (e.g. an in vitro test system using murine cells or in vivo in mice) may not recapitulate such selective activity in a human system (e.g. an in vitro test system using human cells or in vivo in human subjects).

Selective: As used herein, the term “selective” or “selectively binds” is used to refer to a property of an agent to preferentially bind to and/or activate a particular cell type based on a certain property of a population of such cells. In some embodiments, the disclosure provides muteins that are CD25 selective in that such muteins display preferential activation of cells that expressing the orthogonal CD122 receptor relative to the cells expressing the wild-type CD122 receptor. Selectivity is typically assessed by activity measured in an assay characteristic of the activity induced in response to ligand/receptor binding. In some embodiments, IL2 orthologs of the present disclosure possess at least 3 fold, alternatively least 5 fold, alternatively at least 10 fold, alternatively at least 20 fold, alternatively at least 30 fold, alternatively at least 40 fold, alternatively at least 50 fold, alternatively at least 100 fold, alternatively at least 200 fold difference in EC50 on cells expressing the orthogonal CD122 receptor relative to the cells expressing the wild-type CD122 receptor as measured in the same assay.

Significantly Reduced Binding: As used herein, the term “exhibits significantly reduced binding” is used with respect to the affinity of the binding of a variant of a ligand (e.g. an ortholog) to a modified form of a receptor (e.g. an orthogonal CD122) relative to the binding of the variant ligand for the naturally occurring form of a receptor. In some embodiments a ligand (e.g. an ortholog) exhibits significantly reduced binding to the native form of the ligand if the orthogonal ligand binds to the native form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring ligand. Similarly and orthogonal receptor exhibits significantly reduced binding with respect to the native form of the ligand if the native form of the ligand binds to the orthogonal form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring receptor.

Specifically Binds: As used herein the term “specifically binds” refers to the degree of affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the equilibrium dissociation constant between antibody and to the second molecule of the binding pair is greater than about 10⁶M, alternatively greater than about 10⁸ M, alternatively greater than about 10¹⁰ M, alternatively greater than about 10¹¹ M, alternatively greater than about 10¹⁰ M, greater than about 10¹² M as determined by, e.g., Scatchard analysis (Munsen, et al. (1980) Analyt. Biochem. 107:220-239). In one embodiment where the ligand is an IL2 ortholog and the receptor comprises an orthogonal CD122 ECD, the IL2 ortholog specifically binds if the equilibrium dissociation constant of the IL2 ortholog/orthogonal CD122 ECD is greater than about 10⁵M, alternatively greater than about 106 M, alternatively greater than about 10⁷M, alternatively greater than about 10⁸M, alternatively greater than about 10⁹ M, alternatively greater than about 10¹⁰ M, or alternatively greater than about 10¹¹ M. Specific binding may be assessed using techniques known in the art including but not limited to competition ELISA, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET) and surface plasmon resonance assays (see, e.g., Drescher et al., Methods Mol Biol 493:323-343 (2009) with instrumentation commercially available from GE Healthcare Bio-Sciences such as the Biacore 8+, Biacore S200, Biacore T200 (GE Healthcare Bio-Sciences, 100 Results Way, Marlborough Mass. 01752)); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays).

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

Substantially Pure: As used herein in the term “substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

T-cell: As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

Therapeutically Effective Amount: The phrase “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

Transmembrane Domain: The term “transmembrane domain” or “TM” refers to the domain of a membrane spanning polypeptide (e.g. a membrane spanning polypeptide such as hoCD122 or CD132 or a CAR) which, when the membrane spanning polypeptide is associated with a cell membrane, is which is embedded in the cell membrane and is in peptidyl linkage with the extracellular domain (ECD) and the intracellular domain (ICD) of a membrane spanning polypeptide. A transmembrane domain may be homologous (naturally associated with) or heterologous (not naturally associated with) with either or both of the extracellular and/or intracellular domains. In some embodiments the transmembrane domain is the transmembrane domain natively associated with the ECD domain of the cognate receptor from which the orthogonal receptor is derived. In some embodiments the transmembrane domain is the transmembrane domain natively associated with the ICD domain of the cognate receptor from which the orthogonal receptor is derived. In some embodiments the transmembrane domain is the transmembrane domain natively associated with the proliferation signaling domain. In some embodiments the transmembrane domain is the transmembrane domain natively associated with a different protein. Alternatively, the transmembrane domain of the orthogonal receptor may be an artificial amino acid sequence which spans the plasma membrane. In some embodiments, the transmembrane domain of the orthogonal receptor is the transmembrane domain normally associated with the ICD of the cognate receptor from which the orthogonal receptor is derived. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived.

Treat: The terms “treat”, “treating”, treatment” and the like refer to a course of action (such as administering IL2, a CAR-T cell, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

Treg Cell or Regulatory T Cell. The terms “regulatory T cell” or “Treg cell” as used herein refers to a type of CD4⁺ T cell that can suppress the responses of other T cells including but not limited to effector T cells (Teff). Treg cells are characterized by expression of CD4, the a-subunit of the IL2 receptor (CD25), and the transcription factor forkhead box P3 (FOXP3) (Sakaguchi, Annu Rev Immunol 22, 531-62 (2004). By “conventional CD4⁺ T cells” is meant CD4⁺ T cells other than regulatory T cells.

Variant: The terms “protein variant” or “variant protein” or “variant polypeptide” are used interchangeably herein to refer to a polypeptide that differs from a parent polypeptide by virtue of at least one amino acid modification. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. In some embodiments, the variant polypeptide comprises from about one to about ten amino acid modifications relative to the parent polypeptide, alternatively from about one to about five amino acid modifications compared to the parent, alternatively from about one to about three amino acid modifications compared to the parent, alternatively from one to two amino acid modifications compared to the parent, alternatively a single amino acid modification compared to the parent. A variant may be at least about 99% identical to the parent polypeptide, alternatively at least about 98% identical, alternatively at least about 97% identical, alternatively at least about 95% identical, or alternatively at least about 90% identical.

Wild Ty: By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man.

### Variant/Mutein Nomenclature:

In some embodiments, the present disclosure provides variants of wild-type IL2 ligands and CD122 receptors comprising substitutions, deletions, and/or insertions relative to the wt hIL2 and wt hCD122 amino acid sequences, respectively. The residues which are modified in such variant protein may be designated herein by the one-letter or three-letter amino acid code followed by the position of such amino acid in the wild-type protein. For example, in the context of hIL2, “Cys125” or “C125” refers to the cysteine residue at position 125 of wt hIL2. The following nomenclature is used herein to refer to substitutions, deletions or insertions. Substitutions are designated herein by the one letter amino acid code for the wt hIL2 residue followed by the IL2 amino acid position followed by the single letter amino acid code for the new substituted amino acid. For example, “K35A” refers to a substitution of the lysine (K) residue at position 35 of the wt hIL2 sequence with an alanine (A) residue. A deletion is referred to as “des” followed by the amino acid residue and its position in wild-type molecule. For example the term “des-Ala1 hIL2” or “desA1 hIL2” refers to a human IL2 variant comprising a deletion of the alanine at position 1 of wt hIL2. The term “numbered in accordance with hIL2” as used herein refers to the identification of a location of particular amino acid with reference to the position at which that amino acid normally occurs in the mature sequence of the mature wild type hIL2. For example R81 refers to the eighty-first amino acid, arginine, that occurs in the wild type human IL2 sequence. Similarly, the term numbered in accordance with hCD122 as used herein refers to the identification of a location of particular amino acid with reference to the position at which that amino acid normally occurs in the consensus sequence of the mature wild type hCD122.

### DISCUSSION

Adoptive cell therapy, in particular therapy with tumor infiltrating lymphocytes (TILs) is documented as a therapeutic modality having efficacy in the treatment of neoplastic disease in human subjects. See, e.g., Rosenberg (U.S. Pat. No. 5,126,132 issued Jun. 30, 1992) and Spiess, et al (1987) J Natl Cancer Inst 79:1067-1075. In current practice, human TIL therapy consists of ex vivo expansion of TILs obtained from tumor biopsy or resected tumor tissue and reinfusion of the expanded cell population following a lymphodepleting preparative regimen and subsequent support of interleukin-2 (IL-2). The lymphodepleting preparative regimen depletes Tregs and removes cellular “sinks.” The systemic administration of IL-2 at the time of and subsequent to administration of the TIL cell product supports the persistence of the infused TILs in vivo. In typical clinical practice, shortly after infusion of the TILS, the patient receives i.v. high-dose IL-2 (720,000 IU/kg every 8 h until maximal tolerance. This subsequent support with IL-2 is thought to further enhance the survival and clinical efficacy of TIL.

Nevertheless, TIL treatment is associated with significant toxicities. These toxicities arise primarily from the lymphodepleting preparative regimens resulting in pancytopenia and febrile neutropenia and the supportive therapy with high dose IL-2 following re-administration of the enriched TIL cell population. The effect of high dose IL2 typically used in supportive regimens is documented to result in significant toxicities. The most prevalent side effects seen in arising from the use of IL-2 supportive therapy following adoptive cell transfer (ACT) include chills, high fever, hypotension, oliguria, and edema due to the systemic inflammatory and capillary leak syndrome as well as reports of autoimmune phenomena such as vitiligo or uveitis.

The ex vivo expansion of TILs is performed in the presence of high dose IL2 for a significant period of time. IL-2 promotes proliferation and expansion of activated T lymphocytes, potentiates B cell growth, and activates monocytes and natural killer cells. During the TIL expansion process, there is an interclonal competition with different T-cell clones increasing or decreasing in frequency. As it is desirable that the final TIL product to be administered be as enriched as possible for the tumor-specific clones, the non-specific nature of hIL2 fails to provide selective support for the tumor-specific, antigen-experienced T cell clones and it is possible that the most efficacious tumor reactive T cell clones will be out-competed and diluted during the ex vivo expansion phase due to the non-specific T-cell proliferative effects of hIL2.

Additionally, the degree of T-cell differentiation of the T cells following ex vivo stimulation procedures can affect the survival, proliferative capacity and efficacy of the TILs in vivo following reinfusion. Li, et al. (2010) J Immunol. 2010; 184: 452-465. However, IL2 is a very potent molecule and the effects of the exposure of culture TILs to high dose IL2 has been associated with terminal differentiation of the T cells cultured in its presence ex vivo as well as mediation of autoimmunity and transplant rejection in addition to other side effects in vivo.

Consequently, there is need in the art to enable the selective expansion of the tumor antigen-experienced T cells population ex vivo without driving the desired population of these tumor antigen experienced T cells toward differentiation and/or exhaustion. As Li, et al. state:
A key question emerging from our studies is whether using other methods of performing the REP can yield post-REP T cells with a “younger” phenotype associated with maintenance of CD28 expression and other effector-memory markers that are capable of better persistence in vivo during ACT. In other words, can we have the best of both worlds by generating high numbers of tumor-reactive cytotoxic T cells while maintaining a memory phenotype favorable for continued cell division and long-term survival in vivo?

Li, et al at page 465. Furthermore, Li, et al suggest the state of terminal differentiation of the TILs resulting from with current ex vivo protocols involving IL2 is an issue for current TIL therapy and suggest that the potential use of other cytokines such as IL-15 or IL-21 to avoid the effects of IL-2 in the ex vivo preparation of TILs. Additionally, support of TILs with high dose IL2 following ACT is associated with improved therapeutic outcome. However, high dose IL2 therapy is associated with significant toxicity in human subjects and, as previously noted, is one of the major challenges facing TIL therapy.

Similarly, the successes with chimeric antigen receptor (CAR) T cell therapy in early clinical trials involving patients with pre-B cell acute lymphoblastic leukaemia (ALL) or B cell lymphomas was revolutionary and suggested the possibility of curative option for patients who are refractory to standard treatments. These early trials rapid FDA approvals of anti-CD19 CAR T cell products for both acute lymphocytic leukemia (ALL) and certain types of B cell lymphoma.

However, growing experience with these agents has revealed that remissions will be brief in a substantial number of patients owing to poor CAR T cell persistence resulting in relapse. and/or cancer cell resistance resulting from antigen loss or modulation. The administration of IL2 at doses that can be tolerated by the patient fail to provide long term selective maintenance of an activated population of the adoptively transferred cells leading to relapse and recurrence of the neoplastic disease.

The present disclosure provides methods and compositions that overcome these shortcomings and open up new opportunities for the use of adoptive cell therapies including CAR T therapy and Currently the most significant barrier to barrier to the success of adoptive cell therapy and CAR-T therapy in particular is the high rate of disease relapse due to the poor persistence of engineered cell CAR T cells.

A series of experiments were performed to demonstrate the advantages of the orthogonal system in the context of adoptive cell therapy. The results of these experiment demonstrate that use of an orthogonal ligand/receptor system provides multiple benefits over conventional therapies including but not limited to:
(a) the ability to extend the persistence of an orthogonal engineered immune cell (e.g. a orthogonal CAR-T cell) in vivo in a mammalian subject in conjunction with the administration to the subject of an effective amount of an orthogonal ligand;
(b) the ability to specifically and selectively activate and/or induce the proliferation orthogonal immune cells (e.g. a orthogonal CAR-T cell) in vivo in a mammalian subject by administering to the mammalian subject an effective amount of an orthogonal immune cell in conjunction with the administration of an effective amount of an orthogonal ligand;
(c) the ability to restore the activity of an exhausted orthogonal immune cell in a mammalian subject by the administration of an effective amount of an orthogonal ligand;
(d) the ability to prevent relapse in the treatment of a neoplastic disease with a CAR-T cell therapy by the administration of orthogonal CAR-T cell followed by the periodic administration of an effective amount of an cognate orthogonal ligand.

Improved persistence of the therapeutic cells modified to express the orthogonal receptor (e.g. hoCART, hoTIL) by the ability to selectively and potently activate the orthogonal cells because the orthogonal ligand does not provide non-specific off-target toxicity such as that observed with the administration of non specific T cell proliferative agents such as hIL2 which is a well documented source of toxicity in adoptive cell therapy protocols.

The present disclosure is directed to a recombinantly modified immune cell comprising a nucleic acid sequence encoding a receptor comprising an orthogonal extracellular domain and a nucleic acid sequence encoding an orthogonal ligand that exhibits specific binding for the orthogonal extracellular domain of the receptor.

In one embodiment, the present disclosure provides a recombinantly modified mammalian cell comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

In some embodiments, the recombinantly modified mammalian cell is a recombinantly modified immune cell. In some embodiments, the recombinantly modified immune cell is stem cell or a cell of lymphoid origin including but not limited to B cells, T cells, Natural Killer (NK) cells, NKT cells, cytotoxic T lymphocytes (CTLs), regulatory T cells (Tregs), dendritic cells, killer dendritic cells, and mast cells. inflammatory T-lymphocytes, cytotoxic T-lymphocytes, regulatory T-lymphocytes or helper T-lymphocytes including tumor infiltrating lymphocytes (TILs), CD4+ T-lymphocytes and CD8+ T-lymphocytes, cytotoxic T lymphocytes (CTLs), a regulatory T cell (Tregs), including subsets of CD8+ T lymphocytes of various phenotypes including T effector memory phenotype (Tem), T central memory phenotype (Tcm), terminally differentiated Tcm and Tem cells that express CD45RA (Temra), tissue resident memory (Trm) cells, and peripheral memory (Tpm) cells.

In some embodiments the recombinantly modified mammalian cell is a targeting redirected immune cell. By targeting redirected immune cell in the context of the present disclosure as an recombinantly modified immune cell that expresses a non-native molecule on the surface of the cell, the non-native molecule exhibiting specific binding for molecule on the surface of a second cell so that the modified immune cell now binds to the second cell by virtue of the action of the non-native cell surface molecule expressed on the immune cell. In many instances, the non-native surface molecule is an antibody or antibody fragment (scFv) that has specific binding for a tumor antigen expressed on a second (tumor) cell such that the modified immune cell binds to the tumor cell for which the immune cell would otherwise have low affinity. Examples of such targeting redirected immune cells include but are not limited to CAR-T cells and TCR-engineered cells. In some embodiments the immune cell is a CAR-T cell wherein the targeting domain of the CAR-T cell exhibits specific binding to a tumor antigen of a hematopoietic or solid tumor cell. Example of such targeting domains include antibodies, as discussed below, that exhibit specific binding to one or more tumor antigens selected from the group consisting of ROR1, CD4, CD7, B-cell maturation antigen, mesothelin GPC3, c-Met, carbonic anhydrase IX (CA1X), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, an antigen of a cytomegalovirus (CMV) infected cell (e.g., a cell surface antigen), epithelial glycoprotein2 (EGP 2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptor-a, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1CAM), melanoma antigen family A, 1 (MAGE-AI), Mucin 16 (Muc-16), Mucin 1 (Muc-1), NKG2D ligands, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), and a combination thereof. In some embodiments, the immunoresponsive cell expresses one or more adhesion molecules. The adhesion molecule can increase the avidity of the CAR. In some embodiments, the adhesion molecule is selected from the group consisting of CD2, VLA-4, and combinations thereof.

Although CAR-T cell therapy has demonstrated significant efficacy in the treatment of hematopoietic cancers, the use of CAR-T cells in the treatment of solid tumors has not met with similar success. In the absence of a stimulation, CAR-T cells begin to falter after a relatively short time of approximately 2-3 days. In the treatment of blood cancers, there is an abundance of factors to maintain the stimulation of the CAR-T cells. However, the treatment of solid tumors is a significantly different matter. The concentration of tumor antigens to provide stimulus to the CAR-T cells is significantly lower in concentrated solid tumors in contrast the diffuse hematopoietic cancers. Similarly, once the CAR-T cells reaches its tumor target, it is less accessible to factors necessary to maintain the CAR-T cell. Additionally, the solid tumor microenvironment is a harsh zone for the survival of immune cells. For example, the tumor microenvironment (TME) contains an extensive fibrous matrix with immunosuppressive cells that impede immune cell attack and protect the tumor. In the case of the pancreatic cancer TME, there is an increased concentration of hyaluran which prevents the infiltration of immune cells. Furthermore, immune checkpoint receptors on tumor cells can inhibit T cells by binding to regulatory ligands on T cells. The solid tumor TME is further characterized by a nutrient poor, hypoxic and acidic environment which leads to T cell anergy, exhaustion and senescence which make it difficult for the CAR-T cell to survive and proliferate sufficient to substantially impede the proliferation of the tumor. T-cell survival and proliferation under such conditions is challenging.

The present disclosure provides engineered immune cells that encode survival and highly selectively proliferation signals (IL-2 orthologs) to locally stimulate engineered immune cells expressing a modified receptor protein comprising an engineered hCD122 sequence that enables selective stimulation of the CAR-T cell engineered to express the engineered receptor. In one embodiment, the present invention provides a recombinantly modified tumor targeted immune cell that both expresses a ligand that provides selective stimulation of the engineered tumor targeted cell by binding to a modified form of the CD122 protein.

As previously discussed, wild-type hIL2 is associated with significant toxicity in humans. Consequently, it is not desirable to administer an engineered ell that constitutively expresses and secretes large quanties of hIL2 into the bloodstream as this would likely result in significant toxicity. The orthogonal system described by Sockolosky and Garcia (supra) addresses many of these concerns. It provides for a means to selectively ennervate engineered T cells expressing the orthogonal CD122 receptor by the administration of a cognate orthogonal ligand. While this system is highly effective, in certain circumstances it may be useful for the engineered cell to encode its own survival enhancing factor factors. Since the orthogonal hCD122/hoIL2 system provides for selective T-cell stimulation of cells expressing the receptor when contacted by the ligand (not limited to CAR-T cells, but also for other types of engineered immune cells such as TCRs, TILs, etc.), this enables the engineered T-cell to “manufacture its own fuel” to facilitating its survival in the harsh TME. Additionally, systemic administration of the orthogonal ligand presents a challenge to deliver effective amounts of the ligand to the solid tumor environment sufficient to continuously supply the needs of the engineered immune cell. By having the immune cell express both receptor and the ligand it enables selective automated proliferation of the CAR-T cell which incorporates the nucleic acid sequences encoding which may be distant by avoiding the necessity to supply an exogenous growth factor (i.e. the orthogonal ligand, under these conditions call for a CAR-T cell that can withstand the environment. There present disclosure provides for engineered CAR-T cells which express an orthogonal ligand receptor system derived from the IL2 signaling system that is designed to provide its own source of selective cellular stimulation to counter the effects of the repressive environment which facilates a longer duration of action at the tumor site.

In some embodiments, the expression of the orthogonal CD122 ECD receptor of FORMULA #1 and the orthogonal hoIL2 ligand of the FORMULA #2 is under control of a regulated expression system which is responsive to the conditions of the TME to provide enhanced proliferative activity of the engineered CAR-T in the TME as well as potentially provide additional stimulation and support to other engineered CAR-T cells in the TME.

In some embodiments, it is desirable to effect expression of the orthogonal ligand and/or orthogonal receptor in certain tissues or environments, under control of an inducible promoter. A wide variety of tissue selective promoters are known in the art. The term “inducible promoter” refers to promoters that facilitate transcription of the Bioactive polypeptide preferably (or solely) under certain conditions and/or in response to external chemical or other stimuli. Examples of inducible promoters are known in the scientific literature (see, e.g., Yoshida et al., Biochem. Biophys. Res. Comm., 230:426-430 (1997); Iida et al., J. Virol., 70(9): 6054-6059 (1996); Hwang et al., J. Virol., 71(9): 7128-7131 (1997); Lee et al., Mol. Cell. Biol., 17(9): 5097-5105 (1997); and Dreher et al., J. Biol. Chem., 272(46): 29364-29371 (1997). Examples of radiation inducible promoters include the EGR-1 promoter. Boothman et al., volume 138, supplement pages S68-S71 (1994). In some embodiments the promoter is a tissue specific promoter. In some embodiments the promoter is a tumor specific promoter. Tissue specific promoters and tumor specific promoters are well known in the art, e.g., pancreas specific promoters (Palmiter et al., Cell, 50:435 (1987)), liver specific promoters (Rovet et al., J. Biol. Chem., 267:20765 (1992); Lemaigne et al., J. Biol. Chem., 268:19896 (1993); Nitsch et al., Mol. Cell. Biol., 13:4494 (1993)), stomach specific promoters (Kovarik et al., J. Biol. Chem., 268:9917 (1993)), pituitary specific promoters (Rhodes et al., Genes Dev., 7:913 (1993)), and prostate specific promoters (Henderson et. al., U.S. Pat. No. 5,698,443, issued Dec. 16, 1997).

As previously discussed, the conditions of the TME are characterized by hypoxia and low pH. Promoters active in the hypoxic and low pH environments may be used to achieve selective expression and activation in the solid tumor TME.

Additionally, the control of the expression of the orthogonal ligand of FORMULA #2 may be under the control of promoter (e.g. NFAT) that is activated upon binding of the CAR targeting domain to its ligand. See, for example Uchibori, et al. Functional Analysis of an Inducible Promoter Driven by Activation Signals from a Chimeric Antigen Receptor (2019) Molecular Therapy: Oncolytics Vol. 12:16

The currently clinical management of solid tumors is primarily addressed by the administration of chemotherapeutic agents which have significant cellular toxicity including to the engineered immune cells described herein. As it may be desirable in some circumstances to maximize the effect of the co-administration of the engineered immune cells of the present disclosure in combination with chemotherapeutic agents, it is desirable in some circumstances to further recombinantly modify the engineered immune cell to express a drug resistance gene, the drug resistance gene operably linked to an expression control sequence operable in the engineered immune cell. Examples of chimeric antigen receptor T cells expressing drug resistance genes is described in Valton, et al. United States Patent Publication No. US2018/0000914A1 published Jan. 4, 2018 the teaching of which is incorporated by reference.

In another embodiment, the present disclosure provides a recombinant vector encoding: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2. In some embodiments, the first and second nucleic acid sequence are operably linked to an expression control sequence operable in the target recombinantly modified immune cell such that expression of the first and second nucleic acid sequences are under control of a single expression control sequence (i.e. a bicistronic expression cassette). In some embodiments, the first and second nucleic acid sequence are operably linked to an expression control sequence operable in the target recombinantly modified immune cell such that expression of the first and second nucleic acid sequences are under control of a single expression control sequence and the first and second nucleic acid sequences are linked by nucleic acid sequence corresponding to mRNA a ribosome skipping site such as the picornaviral 2A sequence (See, e.g. Funston, et al. (2008) Journal of General Virology 89:389-396) or internal ribosome entry site (IRES) sequence. In some embodiments, when the immune cell is a CAR-T cell, the vector may be polycistronic encoding the CAR, the nucleic acid sequence encoding the orthogonal receptor and the orthogonal ligand are under the control of a single expression control sequence. In other embodiments, one or more of the nucleic acid sequences may be under the control of separate expression control sequences. When expressing multiple polypeptides as in the practice of the present invention, each polypeptide may be operably linked to an expression control sequence (monocistronic) or multiple polypeptides may be encoded by a polycistronic construct where multiple polypeptides are expressed under the control of a single expression control sequence. Examples of elements which may be employed to facilitate polycistronic expression internal ribosome entry site (IRES) elements or the foot and mouth disease virus protein 2A (FMVD2A) system. A wide variety of IRES sites are known (see e.g. Doudna J A, Sarnow P. Translation initiation by viral internal ribosome entry sites. In: Translational Control in Biology and Medicine: Mathews et al, Ed. Cold Spring Harbor, N.Y.: Cold Spring Harbor Laboratory Press; 2007. pp. 129-154; http://www.IRESite.org). Examples of IRES elements include the picornavirus IRES of poliovirus, rhinovirus, encepahlomyocardits virus, the aphthovirus IRES of foot and mouth disease virus, the IRES cricket paralysis virus (CrPV) the hepatitis A IRES of hepatitis A virus, the hepatitis C IRES of hepatitis C virus, the pestivirus IRES of swine fever or bovine diarrhea viruses, the cripavirus IRES, and mammalian IRES elements such as the fibroblast growth factor-1 IRES, the fibroblast growth factor-2 IRES, PDGF IRES, VEGF IRES, IGF-2 IRES. The use of IRES elements typically results in significantly lower expression of the second protein of the polycistronic message. The use of the FMDV2A system results in more efficient production of the downstream proteins as the multiple proteins are first expressed as a fusion protein which contains the autoproteolytic FMDV2A domain which cleaves the polyprotein into functional subunits. Ryan and Drew (1994) EMBO J. 13(4):928-933. Depending on the construction of the polycistronic coding sequence, especially to facilitate restriction endonuclease sites, the use of the FMDV2A system frequently may in the addition of a small number amino acids to the carboxy terminus of the upstream protein.

In some embodiments, the first and second nucleic acid sequence are operably linked to individual expression control sequences, such sequences each being operable in the recombinantly modified immune cell such that expression of the first and second nucleic acid sequences are under control of a separate expression control sequence. In some embodiments, the first and second nucleic acid sequence are operably linked to individual expression control sequences, such sequences each operable in the target recombinantly modified immune cell such that expression of the first and second nucleic acid sequences are under control of a separate expression control sequence wherein the first and second expression control sequences are different.

In some embodiments, the expression control sequence is selected from the group consisting of constitutively active, selectively active, and regulated expression control sequences as more fully described herein..

The present disclosure further provides vectors comprising the nucleic acids encoding the hoCD122 ECD receptor and the hoIL2 ligand and associated expression control sequences and nucleic acid molecules encoding functions that are desired in the engineered immune cell such as drug resistance genes, targeting ligands, chimeric antigen receptor sequences, engineered TCR sequences, etc. In some embodiments, the vector is a viral vector. In some embodiments the viral vector is selected from the group consisting of retroviral vectors and lentiviral vectors.

The present disclosure provides a recombinant viral vector comprising a first nucleic acid sequence encoding a receptor polypeptide, the receptor polypeptide comprising an intracellular domain, a transmembrane domain, and an extracellular domain, wherein the extracellular domain of said receptor comprises a polypeptide of the formula 1, and a second nucleic acid sequence encoding a ligand polypeptide that specifically binds to the extracellular domain of the receptor polypeptide, the ligand polypeptide having an amino acid sequence of the formula 2.

As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (e.g., recombinantly engineered to replicate under certain conditions) or replication deficient (e.g. substantially incapable of replication in the absence of a cell line capable of complementing the deleted functions of the virus). The viral vector can possess certain modifications to make it “selectively replicating,” i.e. that it replicates preferentially in certain cell types or phenotypic cell states.

Viral vector systems useful in the practice of the instant invention include, for example, naturally occurring or recombinant viral vector systems. Examples of viruses useful in the practice of the present invention include recombinantly modified enveloped or non-enveloped DNA and RNA viruses. For example, viral vectors can be derived from the genome of human or bovine adenoviruses, vaccinia virus, lentivirus, herpes virus, adeno-associated virus, human immunodeficiency virus, sindbis virus, and retroviruses (including but not limited to Rous sarcoma virus), and hepatitis B virus.

Typically, genes of interest are inserted into such vectors to allow packaging of the gene construct, typically with accompanying viral genomic sequences, followed by infection of a sensitive host cell resulting in expression of the gene of interest (e.g. a targeting antigen). Additionally, the expression vector encoding the anti-targeting antigen CAR may also be an mRNA vector.

When a viral vector system is to be employed for transfection, retroviral or lentiviral expression vectors are preferred to transfect T-cells due to an enhanced efficacy of gene transfer to T-cells using these systems resulting in a decreased time for culture of significant quantities of T-cells for clinical applications. In particular, gamma retroviruses a particularly preferred for the genetic modification of clinical grade T-cells and have been shown to have therapeutic effect. Pule, et al. (2008) Nature Medicine 14(11):1264-1270.

Similarly, self-inactivating lentiviral vectors are also useful as they have been demonstrated to integrate into quiescent T-cells. June, et al. (2009) Nat Rev Immunol 9(10):704-716. Particular retroviral vectors useful in the expression of CAR sequences (and optional additional transgenes) are those described in Naldini, et al. (1996) In Vivo Gene Delivery and Stable Transduction of Nondividing Cells by a Lentiviral Vector, Science 272: 263-267; Naldini, et al. (1996) Efficient transfer, integration, and sustained long-term expression of the transgene in adult rat brains injected with a lentiviral vector, Proc. Natl. Acad. Sci. USA Vol. 93, pp. 11382-11388; Dull, et al. (1998) A Third-Generation Lentivirus Vector with a Conditional Packaging System, J. Virology 72(11):8463-8471; Milone, et al. (2009) Chimeric Receptors Containing CD137 Signal Transduction Domains Mediate Enhanced Survival of T Cells and Increased Antileukemic Efficacy In Vivo, Molecular Therapy 17(8):1453-1464; Kingsman, et al. U.S. Pat. No. 6,096,538 issued Aug. 1, 2000 and Kingsman, et al. U.S. Pat. No. 6,924,123 issued Aug. 2, 2005 herein incorporated by reference. In one embodiment of the invention, the CAR expression vector is a Lentivector® lentiviral vector available under license from Oxford Biomedica.

In some embodiments, the present disclosure provides recombinantly modified cells expressing orthogonal receptors, the orthogonal receptor having an extracellular domain that specifically binds to a cognate orthogonal ligand, a transmembrane domain and an intracellular domain. In some embodiments, the orthogonal receptor is a receptor comprising an ECD of FORMULA #1. In some embodiments, the orthogonal receptor comprising an ECD having an amino acid sequence of SEQ ID NO. 1. In some embodiments, the orthogonal receptor comprises the extracellular domain of FORMULA #1 and an intracellular domain (ICD) corresponding to the ICD of hCD122. In some embodiments, the orthogonal receptor comprises the extracellular domain of FORMULA #1 and the transmembrane and intracellular domains (ICD) corresponding to the transmembrane and ICD domains of hCD122. In some embodiments the orthogonal receptor comprises the amino acid sequence of SEQ ID NO. 2. In some embodiments, the intracellular domain of the orthogonal receptor further comprises at least one STAT3 binding motif.

In some embodiments, the present disclosure provides recombinantly modified cells comprising at least one nucleic acid sequences encoding an hIL2 ortholog wherein said at nucleic acid is encoded by a viral vector. In some embodiments, the orthologs are human IL2 orthologs. In some embodiments, the IL2 orthologs are ligands for a receptor comprising an ECD of FORMULA #1. In some embodiments, the hIL2 orthologs are ligands for an orthogonal receptor comprising the extracellular domain of CD122 comprising amino acid substitutions at positions H133 and Y134. In some embodiments, the IL2 orthologs are ligands for an orthogonal receptor comprising the extracellular domain of CD122 comprising amino acid substitutions at positions H133 and Y134. In some embodiments, the IL2 orthologs are ligands for an orthogonal hCD122 comprising amino acid substitutions at positions H133 and Y134. In some embodiments, the IL2 orthologs are ligands for an orthogonal hCD122 comprising the amino acid substitutions H133D and Y134F (SEQ ID NO: 1).

In some embodiments, the present disclosure provides immune cells that are recombinantly modified to express an orthogonal receptor. In some embodiments, the present disclosure provides mammalian cells that are recombinantly modified to express a receptor comprising the ECD of an orthogonal CD122. In some embodiments, the present disclosure provides mammalian cells that are recombinantly modified to express an orthogonal receptor comprising the extracellular domain of CD122 comprising amino acid substitutions at positions H133 and Y134. In some embodiments, the present disclosure provides mammalian cells that are recombinantly modified to express a receptor comprising the extracellular domain of orthogonal hCD122 comprising amino acid, said ECD comprising the substitutions at positions H133D and Y134F (SEQ ID NO:1). In some embodiments, the present disclosure provides mammalian cells that are recombinantly modified to express an orthogonal hCD122 comprising the amino acid substitutions H133D and Y134F (SEQ ID NO:2).

In some embodiments, the present disclosure provides a method of causing a proliferative response in a mammalian immune cell, the cell having been recombinantly modified to express: (1) a receptor protein comprising an intracellular domain, a transmembrane domain and an extracellular domain (ECD), the extracellular domain comprising a polypeptide of the of FORMULA #1, and (2) a polypeptide ligand for the receptor protein comprising the (a) a secretion leader sequence (signal sequence) and (b) a polypeptide comprising the amino acid sequence of FORMULA #2 such that the polypeptide of the FORMULA #2 is secreted from the engineered immune cell and is capable of contacting and specifically binding to the ECD of the receptor protein, and by such contacting, activates the signaling cascade of the ICD of the receptor.

In some embodiments, the present disclosure provides a method of causing a proliferative signaling response in a mammalian immune cell, the cell having been recombinantly modified to express: (1) a receptor protein comprising an intracellular domain, a transmembrane domain and an extracellular domain (ECD), the extracellular domain comprising a polypeptide of the of FORMULA #1; and (2) a polypeptide ligand for the ECD of the receptor protein comprising, the polypeptide ligand comprising a fusion protein of membrane anchoring sequence and polypeptide comprising the amino acid sequence of FORMULA #2 such that a polypeptide comprising the amino acid sequence of of the FORMULA #2 is displayed (tethered) on the surface from the engineered immune cell. When the fusion protein comprising the polypeptide ligand is displayed (tethered) to the surface of the engineered mammalian immune cell, the ligand domain of the fusion protein is capable of contacting and specifically binding to the ECD of the receptor protein and activates the signaling cascade defined by of the ICD of the receptor. Sequences useful in the construction of the fusion protein to achieve tethered surface display of the polypeptide ligand of FORMULA #2 are known in the art.

The present disclosure further provides a method activation of proliferation of a mammalian immune cell expressing a receptor comprising an orthogonal ECD of FORMULA #1 with an effective amount of an ortholog of FORMULA #2. In some embodiments, for example where the expression orthogonal ligand encoded by the engineered cell is under the control of expression control sequences that are activated preferentially in vivo (e.g. where the nucleic acid sequence encoding ligand of FORMULA #2 is under control of a promoter activated in response to the tumor microenvironment), it may be desirable to contact the engineered the cells ex vivo (e.g. in preparing the cell product for administration to ex vivo stimulation or proliferation) or in vivo (e.g. contemporaneously with and for period of time after administration of the engineered cell product) with a hIL2 ortholog polypeptide of the FORMULA #2 to facilitate the administration of an activated engineered cell population and support the administered cell population in he subject for a period of time after administration ensure sufficient proliferative signaling for the engineered immune cells before they begin (or “ramp up”) autonomous expression of the orthogonal ligand and concomitant autonomous activation of the engineered immune cell expressing the receptor comprising the orthogonal ECD comprising the amino acid sequence of FORMULA #1.

The present disclosure further provides a method of effecting a response in a mammalian immune cell expressing a receptor comprising an orthogonal ECD of FORMULA #1 by contacting said immune cell with an effective amount of a cognate ortholog of SEQ ID NO:3 or SEQ ID NO:4 ex vivo and/or in vitro in an amount sufficient to effect intracellular signaling from the ICD of the receptor of SEQ ID NO:2. The present disclosure provides a method of causing a response in a mammalian immune cell expressing a receptor of SEQ ID NO:2, the method comprising contacting said receptor with an effective amount of a polypeptide of SEQ ID NO: 3, wherein said method is practiced ex vivo.

In some embodiments, the present disclosure provides methods of use comprising the use a first hIL2 ortholog of FORMULA #2 (i.e., orthogonal hIL2 ligand) ex vivo and a second hIL2 ortholog of FORMULA #2 in vivo. In some embodiments, the present disclosure provides methods of use comprising the use a first hIL2 ortholog of FORMULA #2 ex vivo and a second hIL2 ortholog of FORMULA #2 in vivo, wherein the first hIL2 ortholog of FORMULA #2 and the second hIL2 ortholog of FORMULA #2 are the same orthologs or different orthologs. In some embodiments, the present disclosure provides methods of use of hIL2 orthologs of FORMULA #2 to cause the proliferation of a mammalian immune cell expressing a receptor comprising an orthogonal ECD of FORMULA #1. In some embodiments, the present disclosure provides methods of use of hIL2 orthologs of FORMULA #2 to cause the activation of a mammalian cell expressing a mammalian immune cell expressing a receptor comprising an orthogonal ECD of FORMULA #1. In some embodiments, the present disclosure provides methods of use of hIL2 orthologs of FORMULA #2 ex vivo and/or in vivo to cause the proliferation of a mammalian immune cell expressing a receptor comprising an orthogonal ECD of FORMULA #1. In some embodiments, the present disclosure provides methods of use of hIL2 orthologs of FORMULA #2 to cause the activation of a mammalian cell recombinantly modified to express an orthogonal receptor comprising the extracellular domain of hCD122 comprising amino acid substitutions at of SEQ ID NO: 1. In some embodiments, the present disclosure provides methods of use of hIL2 orthologs of SEQ ID NO: 3 or SEQ ID NO: 4 to cause the activation of a mammalian cell recombinantly modified to express an orthogonal receptor comprising the amino acid substitutions H133D and Y134F (SEQ ID NO:2).

In some embodiments, the present disclosure provides methods for the preparation of a population of recombinantly modified mammalian immune cells, the immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

The present disclosure further provides methods for the preparation of a population of recombinantly modified mammalian immune cells, said immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2, and nucleic acid sequence encoding a chimeric antigen receptor.

The present disclosure further provides a cell therapy product comprising a pharmaceutically acceptable formulation of population of recombinantly modified mammalian immune cells, said immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

The present disclosure further provides a cell therapy product comprising a pharmaceutically acceptable formulation of population of recombinantly modified mammalian immune cells, said immune cells comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2, and nucleic acid sequence encoding a chimeric antigen receptor.

The present disclosure further provides methods of preparing a pharmaceutically acceptable dosage form of a cell therapy product comprising at least one (alternatively 2, 3, 4 or more) species of engineered immune cells that express a transmembrane receptor protein wherein the extracellular domain of such transmembrane receptor protein comprises the extracellular domain of an CD122 orthogonal polypeptide of FORMULA #1, a secreted form of an IL2 ligand of FORMULA #2, and a chimeric antigen receptor, wherein the fraction of engineered cells in the cell therapy product comprises at least 30%, alternatively at least 40%, alternatively at least 50%, alternatively at least 60%, alternatively at least 70%, alternatively at least 80%, or alternatively at least 90% of the total number of cells in the cell therapy product.

In some embodiments a therapeutic method is provided, the method comprising introducing into a subject in need thereof of pharmaceutically acceptable formulation comprising a population of engineered allogenic or autologous immune cells allogeneic that express: (1) a transmembrane receptor polypeptide wherein the extracellular domain of the transmembrane receptor polypeptide comprises the extracellular domain of an CD122 orthogonal polypeptide of FORMULA #1; (2) a secreted or membrane tethered form of an IL2 ligand of FORMULA #2; and (3) a chimeric antigen receptor.

In some embodiments, the compositions and methods of the present disclosure comprise the step of genetically modifying a human immune cell by using at least one endonuclease to facilitate incorporate the modifications of to the ECD of the orthogonal hCD122 of FORMULA #1 into the genomic sequence of the human immune cell. As used herein, the term “endonuclease” is used to refer to a wild-type or variant enzyme capable of catalyzing the cleavage of bonds between nucleic acids within a DNA or RNA molecule, preferably a DNA molecule. The endonucleases of the present disclosure are sequence specific in that they recognize and cleave the nucleic acid molecules at specific “target” sequences. Endonucleases are often categorized with respect to the degree of specificity and sequence identity characteristic of the target sequences. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogeneous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23(3):967-973, a TALE-nuclease, a Cas9 endonuclease from CRISPR system as or a modified restriction endonuclease to extended sequence specificity (Eisenschmidt, et al. 2005; 33(22): 7039-7047). In some embodiments of the invention, the immune cell (e.g. a CAR-T expressing the orthogonal receptor ECD of FORMULA #1) is modified to reduce alloreactivity through inactivation of one more components of the T-cell receptor (TCR). Methods for such modification of T cells is described in Galetto, et al. United States Patent Publication No. US 2013/015884A1 published Nov. 28, 2013 and methods for TCRalpha deficient T-cells by expressing pTalpha resulting in restoration of a functional CD3 complex as described in Galetto, et al. U.S. Pat. No. 10,426,795B2 issued Oct. 21, 2019, the teaching of which is herein incorporated by reference. In one embodiment, the immune cell has at least one CD122 allele converted into a nucleic acid sequence encoding orthogonal hCD122 of with an ECD of FORMULA #1 or SEQ ID NO: 2. In an alternative embodiment, the immune cell has both CD122 alleles converted into a nucleic acid sequence encoding orthogonal hCD122 of with an ECD of FORMULA #1 or SEQ ID NO: 2 such that the immune cell does not express a wild-type CD122 receptor making the proliferation of such cell dependent on the on the supply of an orthogonal ligand of FORMULA #2.

### Nomenclature of Amino Acid Substitutions

The present disclosure provides polypeptide variants. The following nomenclature is used herein to refer to substitutions, deletions or insertions. Residues may be designated herein by the one-letter or three-letter amino acid code of the naturally occurring amino acid found in the wild-type molecule but followed by the IL2 amino acid position, e.g., “Cys125” or “C125” refers to the cysteine residue at position 125 of the wild-type hIL2 molecule. In reference to the IL2 orthologs, substitutions are designated herein by the one letter amino acid code followed by the IL2 amino acid position followed by the one letter amino acid code which is substituted. For example, an IL2 ortholog having the modification “K35A” refers to a substitution of the lysine (K) residue at position 35 of the wild-type IL2 sequence with an alanine (A) residue at this position. A deletion of an amino acid reside is referred to as “des” followed by the amino acid residue and its position. For example, the term “des-Ala1” or “desA1” refers to the deletion of the alanine at position 1 of the polypeptide. Similarly, in reference to amino acid substitutions in the orthogonal hCD122, amino acid substitutions are designated herein by the one letter amino acid code of the naturally occurring amino acid followed by the number of its position in the wild-type mature hCD122 sequence followed by the one letter amino acid code of the amino acid which is substituted at that position. For example, a CD122 ortholog having a substitution of the tyrosine residue at position 134 with a phenylalanine residue, the substitution is abbreviated “Y134F.”

### Ortho Receptor ECD Sequence: FORMULA #1:

A receptor polypeptide comprising an extracellular domain, a transmembrane domain and an intracellular domain, the extracellular domain of said polypeptide comprising an amino acid sequence of the following FORMULA #1 (SEQ ID NO: 36):

Ala-Val-Asn-Gly-Thr-Ser-Gln-Phe-Thr-
Cys-Phe-Tyr Asn-Ser-Arg-Ala-Asn-Ile-
Ser-Cys-Val-Trp-Ser-Gln-Asp-Gly-Ala-
Leu-Gln-Asp-Thr-Ser-Cys-Gln-Val-His-
Ala-Trp-Pro-Asp-Arg-Arg-Arg-Trp-Asn-
Gln-Thr-Cys-Glu-Leu-Leu-Pro-Val-Ser-
Gln-Ala-Ser-Trp-Ala-Cys-Asn-Leu-Ile-
Leu-Gly-Ala-Pro-Asp-Ser-AA70-Lys-Leu-
AA73-Thr-Val-Asp-Ile-Val-Thr-Leu-Arg-
Val-Leu-Cys-Arg-Glu-Gly-Val-Arg-Trp-
Arg-Val-Met-Ala-Ile-Gln-Asp-Phe-Lys-
Pro-Phe-Glu-Asn-Leu-Arg-Leu-Met-Ala-
Pro-Ile-Ser-Leu-Gln-Val-Val-His-Val-
Glu-Thr-His-Arg-Cys-Asn-Ile-Ser-Trp-
Glu-Ile-Ser-Gln-Ala-Ser-AA133-AA134-
Phe-Glu-Arg-His-Leu-Glu-Phe-Glu-Ala-
Arg-Thr-Leu-Ser-Pro-Gly-His-Thr-Trp-
Glu-Glu-Ala-Pro-Leu-Leu-Thr-Leu-Lys-
Gln-Lys-Gln-Glu-Trp-Ile-Cys-Leu-Glu-
Thr-Leu-Thr-Pro-Asp-Thr-Gln-Tyr-Glu-
Phe-Gln-Val-Arg-Val-Lys-Pro-Leu-Gln-
Gly-Glu-Phe-Thr-Thr-Trp-Ser-Pro-Trp-
Ser-Gln-Pro-Leu-Ala-Phe-Arg-Thr-Lys-
Pro-Ala-Ala-Leu-Gly-Lys-Asp-Thr

wherein:

AA70=Gln or Tyr;

AA73=Thr, Asp or Tyr;

AA133=His, Asp, Glu or Lys; and/or

AA134=Tyr, Phe, Glu or Arg.

The ECD of the hCD122 protein comprises several secondary structural features as summarized in the Table 4 below:

TABLE 4
Secondary Structural Features of hCD122 ECD

| | Position In | Position In |
|---|---|---|
| | Precursor form of | Mature form of |
| | hCD122 (including | hCD122 (excluding |
| Feature | 26AA signal sequence) | signal sequence) |
| N-linked glycosylation site | Asn29 | Asn3 |
| Disulfide bond | Cys36-Cys46 | Cys10-Cys20 |
| N-linked glycosylation site | Asn43 | Asn17 |
| Disulfide bond | Cys59-Cys110 | Cys33-Cys84 |
| N-linked glycosylation site | Asn71 | Asn45 |
| Disulfide bond | Cys74-Cys86 | Cys48-Cys60 |
| N-linked glycosylation site | Asn149 | Asn123 |

When making modifications in the ECD sequence of hCD122, it some embodiments, the amino acids involved in the formation of secondary structural features are retained to maintain the secondary structure of the protein. In general, maintenance of disulfide bonds is desirable. In some embodiments deletion of glycosylation sites may be desired. Consequently, one or more conservative amino acid substitutions of asparagine (for example with alanine or isoleucine) at or more of positions 3, 17, 42 and/or 123 of the ECD mature of hCD122 may be incorporated to eliminate one or more these N-linked glycosylation sites.

hoCD122 ICDs with Additional STAT3 Signaling Domains:

In some embodiments, the present disclosure provides methods of use hIL2 orthologs that are cognate ligands of receptors having an ECD corresponding to ECD of orthogonal hCD122 of FORMULA #1. In some embodiments, the IL2 orthologs are ligands for an receptors having an ECD corresponding to ECD of orthogonal hCD122 of FORMULA #1 the ICD of which is further modified to encode one or more STAT3 binding motifs. In some embodiments, the term IL2 orthologs refers to ligands for a receptor comprising the extracellular domain of human CD122 comprising amino acid substitutions at positions H133 and/or Y134 the ICD of which comprises one or more STAT3 binding motifs. In some embodiments, the IL2 orthologs of FORMULA #2 are ligands for a receptor comprising the extracellular domain of human CD122 comprising amino acid substitutions at positions H133 and/or Y134 (SEQ ID NO:10. In some embodiments, the IL2 orthologs are ligands for a receptor comprising the extracellular domain of human CD122 comprising amino acid substitutions at positions H133 and/or Y134 the ICD of which further comprises one or more STAT3 binding motifs. In some embodiments, the IL2 orthologs are ligands for an orthogonal human CD122 comprising amino acid substitutions at positions H133 and Y134. In some embodiments, the IL2 orthologs are ligands for an orthogonal CD122 comprising the amino acid substitutions H133D and Y134F. In some embodiments, the orthologs of the present disclosure are hIL2 orthologs that are cognate ligands of a receptor comprising the extracellular domain of a hCD122 molecule comprising amino acid substitutions at position H133 and Y134 the ICD of which comprises one or more STAT3 binding motifs.

In some embodiments, the orthologs of the present disclosure are hIL2 orthologs that are cognate ligands of a receptor comprising the extracellular domain of a hCD122 molecule comprising and amino acid substitutions at position H133. In some embodiments, the orthologs of the present disclosure are hIL2 orthologs that are cognate ligands of a receptor comprising the extracellular domain of a hCD122 molecule comprising amino acid substitutions at position H133 the ICD of which comprises one or more STAT3 binding motifs. In some embodiments, the orthologs of the present disclosure are hIL2 orthologs that are cognate ligands of a receptor comprising the extracellular domain of a hCD122 molecule comprising amino acid substitutions at position H133D. In some embodiments, the orthologs of the present disclosure are hIL2 orthologs that are cognate ligands of a receptor comprising the extracellular domain of a hCD122 molecule comprising amino acid substitutions at position H133D the ICD of which comprises one or more STAT3 binding motifs. In some embodiments, the orthologs of the present disclosure are hIL2 orthologs that are cognate ligands of a receptor comprising the extracellular domain of a hCD122 molecule comprising and amino acid substitutions at position Y134. In some embodiments, the orthologs of the present disclosure are hIL2 orthologs that are cognate ligands of a receptor comprising the extracellular domain of a hCD122 molecule comprising amino acid substitutions at position Y134 the ICD of which comprises one or more STAT3 binding motifs. In some embodiments, the orthologs of the present disclosure are hIL2 orthologs that are cognate ligands of a receptor comprising the extracellular domain of a hCD122 molecule comprising amino acid substitutions at position Y134F. In some embodiments, the orthologs of the present disclosure are hIL2 orthologs that are cognate ligands of a receptor comprising the extracellular domain of a hCD122 molecule comprising amino acid substitutions at position Y134F the ICD of which comprises one or more STAT3 binding motifs.

In some embodiments, the orthologs useful in the practice of the present disclosure are hIL2 orthologs that are cognate ligands of a receptor comprising the extracellular domain of a hCD122 molecule comprising amino acid substitutions at position Y134 the ICD of which comprises one or more STAT3 binding motifs. In some embodiments, the orthologs of the present disclosure are hIL2 orthologs that are cognate ligands of receptor comprising the extracellular domain of orthogonal human CD122 comprising the amino acid substitutions H133D and Y134F. In some embodiments, the orthologs of the present disclosure are hIL2 orthologs that are cognate ligands of and orthogonal human CD122 comprising the amino acid substitutions H133D and Y134F.

### IL2 Orthologs

In various embodiments, the compositions and methods of the present disclosure comprise the use of nucleic acids encoding IL2 orthologs, recombinantly modified immune cells expressing IL2 orthologs, wherein the IL2 orthologs comprise a polypeptide having an amino acid sequence according to the following FORMULA #2 (SEQ ID NO: 37):
(AA1)-(AA2)-(AA3)-(AA4)-(AA5)-(AA6)-(AA7)-(AA8)-(AA9)-T10-Q11-L12-(AA13)-(AA14)-(AA15)-(AA16)-L17-(AA18)-(AA19)-(AA20)-L21-(AA22)-(AA23)-I24-L25-N26-(AA27)-I28-N29-N30-Y31-K32-N33-P34-K35-L36-T37-(AA38)-(AA39)-L40-T41-(AA42)-K43-F44-Y45-M46-P47-K48-K49-A50-(AA51)-E52-L53-K54-(AA55)-L56-Q57-C58-L59-E60-E61-E62-L63-K64-P65-L66-E67-E68-V69-L70-N71-L72-A73-(AA74)-S75-K76-N77-F78-H79-(AA80-(AA81)-P82-R83-D84-(AA85)-(AA86)-S87-(AA88)-(AA89)-N90-(AA91)-(AA92)-V93-L94-E95-L96-(AA97)-G98-S99-E100-T101-T102-F103-(AA104)-C105-E106-Y107-A108-(AA109)-E110-T111-A112-(AA113)-I114-V115-E116-F117-L118-N119-R120-W121-I122-T123-F124-(AA125)-(AA126)-S127-I128-I129-(AA130)-T131-L132-T133
   wherein:
AA1 is A (wild type) or deleted;
AA2 is P (wild type) or deleted;
AA3 is T (wild type), C, A, G, Q, E, N, D, R, K, P, or deleted
AA4 is S (wild type) or deleted;
AA5 is S (wild type) or deleted;
AA6 is S (wild type) or deleted;
AA7 is T (wild type) or deleted;
AA8 is K (wild type) or deleted;
AA9 is K (wild type) or deleted;
AA13 is Q (wild type), W or deleted;
AA14 is L (wild type), M, W or deleted;
AA15 is E (wildtype), K, D, T, A, S, Q, H or deleted;
AA16 is H (wildtype), N or Q or deleted;
AA18 is L (wild type), R, G, M, F, E, H, W, K, Q, S, V, I, Y, H, D or T;
AA19 is L (wildtype), A, V, I or deleted;
AA20 is D (wildtype), T, S M L, or deleted;
AA22 is Q (wild type), F, E, G, A, L, M, F, W, K, S, V, I, Y, H, R, N, D, T, F or deleted;
AA23 is M (wild type), A, W, H, Y, F, Q, S, V, L, T, or deleted;
AA27 IS G (wildtype), K, S or deleted;
AA38 is R (wild type), W or G;
AA39 is M (wildtype), L or V;
AA42 is F (wildtype) or K;
AA51 is T (wildtype), I or deleted;
AA55 is H (wildtype) or Y;
AA74 is Q (wild type), N, H, S;
AA80 is L (wild type), F or V;
AA81 is R (wild type), I, D, Y, T or deleted;
AA85 is L (wild type) or V;
AA86 is I (wild type) or V;
AA88 is N (wildtype), E or Q or deleted;
AA89 is I (wild type) or V;
AA91 is V (wild type), R or K;
AA92 is I (wild type) or F;
AA97 is K (wild type) or Q;
AA104 is M (wild type) or A;
AA109 is D (wildtype), C or a non-natural amino acid with an activated side chain;
AA113 is T (wild type) or N;
AA125 is C (wild type), A or S;
AA126 is Q (wild type) or H, M, K, C, D, E, G, I, R, S, or T; and/or
AA130 is S (wild type), T or R.

In some embodiments, the present disclosure provides hIL2 orthologs which are hIL2 polypeptides comprising the following sets of amino acid modifications:
[E15S-H16Q-L19V-D20L-Q22K]
[H16N, L19V, D20N, Q22T, M23H, G27K];
[E15D, H16N, L19V, D20L, Q22T, M23H];
[E15D, H16N, L19V, D20L, Q22T, M23A],
[E15D, H16N, L19V, D20L, Q22K, M23A];
[E15S; H16Q; L19V, D20T; Q22K, M23L];
[E15S; H16Q; L19V, D20T; Q22K, M23S];
[E15S; H16Q; L19V, D20S; Q22K, M23S];
[E15S; H16Q; L19I, D20S; Q22K; M23L];
[E15S; L19V; D20M; Q22K; M23S];
[E15T; H16Q; L19V; D20S; M23S];
[E15Q; L19V; D20M; Q22K; M23S];
[E15Q; H16Q; L19V; D20T; Q22K; M23V];
[E15H; H16Q; L19I; D20S; Q22K; M23L];
[E15H; H16Q; L19I; D20L; Q22K; M23T];
[L19V; D20M; Q22N; M23S].

For example, “des-Ala1” means the alanine as position 1 is absent in the IL2 polypeptide. In some embodiments, the IL2 orthologs or the present invention comprise one of the following sets of amino acid modifications:
[E15S-H16Q-L19V-D20L-Q22K-M23A-C125S];
[E15S-H16Q-L19V-D20L-Q22K-C125S];
[E15S-H16Q-L19V-D20L-M23A-C125S];
[E15S-H16Q-L19V-D20L-C125S];
[E15S-H16Q-L19V-D20L-Q22K-M23A-C125A];
[E15S-H16Q-L19V-D20L-M23A-C125A];
[E15S-H16Q-L19V-D20L-Q22K-C125A];
[E15S-H16Q-L19V-D20L-C125A];
[desAla1-E15S-H16Q-L19V-D20L-Q22K-M23A-C125S];
[desAla1-E15S-H16Q-L19V-D20L-Q22K-C125S];
[desAla1-E15S-H16Q-L19V-D20L-C125S];
[desAla1-E15S-H16Q-L19V-D20L-Q22K-M23A-C125A];
[desAla1-E15S-H16Q-L19V-D20L-Q22K-C125A];
[desAla1-E15S-H16Q-L19V-D20L-C125A];
[desAla1-E15S-H16Q-L19V-D20L-Q22K-M23A];
[desAla1-E15S-H16Q-L19V-D20L-M23A];
[desAla1-E15S-H16Q-L19V-D20L-Q22K]; or
[desAla1-E15S-H16Q-L19V-D20L].
   Ortholog Mutations to Increase hoCD122 ECD Affinity

In some embodiments, hIL2 orthologs useful in the practice of the present disclosure contain one or more mutations in positions of the hIL2 sequence that either contact hCD122 or alter the orientation of other positions contacting the ECD of hoCD122, resulting in an IL2 ortholog having increased affinity for the ECD of hoCD122. IL2 residues that have been identified as being involved in the binding of IL2 to the ECD of hoCD122 include L12, Q13, H16, L19, D20, M23, Q74, L80, R81, D84, L85, I86, S87, N88, 189 V91, 192, and E95. In some embodiments, the IL2 ortholog comprises one or more of the amino acid substitutions: Q74N, Q74H, Q74S, L80F, L80V, R81D, R81T, L85V, I86V, I89V, and/or I92F or combinations thereof. In some embodiments, the IL2 ortholog comprises one or more of the amino acid substitutions: L80F, R81D, L85V, I86V and I92F. In some embodiments, the IL2 ortholog comprises one or more of the amino acid substitutions: N74Q, L80F, R81D, L85V, I86V, I89V, and I92F. In some embodiments, the IL2 ortholog comprises one or more of the amino acid substitutions: Q74N, L80V, R81T, L85V, I86V, and I92F. In some embodiments, the IL2 ortholog comprises one or more of the amino acid substitutions: Q74H, L80F, R81D, L85V, I86V and I92F. In some embodiments, the IL2 ortholog comprises one or more of the amino acid substitutions: Q74S, L80F, R81D, L85V, I86V and I92F. In some embodiments, the IL2 ortholog comprises one or more of the amino acid substitutions: Q74N, L80F, R81D, L85V, I86V and I92F. In some embodiments, the IL2 ortholog comprises one or more of the amino acid substitutions: Q74S, R81T, L85V, and I92F. In some embodiments, the IL2 ortholog comprises [L80F-R81D-L85V-I86V-I92F]. In some embodiments, the present disclosure provides hIL2 orthologs which comprise one of the following sets of amino acid modifications:
[E15S-H16Q-L19V-D20L-M23A-L80F-R81D-L85V-I86V-I92F];
[E15S-H16Q-L19V-D20L-Q22K-L80F-R81D-L85V-I86V-I92F];
[E15S-H16Q-L19V-D20L-Q22K-M23A L80F-R81D-L85V-I86V-I92F];
[E15S-H16Q-L19V-D20L-M23A-L80F-R81D-L85V-I86V-I92F-Q126H];
[E15S-H16Q-L19V-D20L-Q22K-L80F-R81D-L85V-I86V-I92F-Q126H];
[E15S-H16Q-L19V-D20L-Q22K-M23A-L80F-R81D-L85V-I86V-I92F-Q126H];
[E15S-H16Q-L19V-D20L-M23A-L80F-R81D-L85V-I86V-I92F-Q126M];
[E15S-H16Q-L19V-D20L-Q22K-L80F-R81D-L85V-I86V-I92F-Q126M]; or
[E15S-H16Q-L19V-D20L-Q22K-M23A-L80F-R81D-L85V-I86V-I92F-Q126M].

In some embodiments, the orthologs comprise the substitution L85V that has been identified as increasing affinity of IL2 to CD122. In some embodiments, the present disclosure provides hIL2 orthologs which are hIL2 polypeptides comprising one of the following sets of amino acid modifications:
[E15S-H16Q-L19V-D20L-M23A-L85V];
[E15S-H16Q-L19V-D20L-Q22K-M23A-L85V];
[E15S-H16Q-L19V-D20L-M23A-L85V];
[E15S-H16Q-L19V-D20L-Q22K-M23A-L85V];
[E15S-H16Q-L19V-D20L-M23A-L85V-Q126H];
[E15S-H16Q-L19V-D20L-Q22K-M23A-L85V-Q126H];
[E15S-H16Q-L19V-D20L-M23A-L85V-Q126M]; or
[E15S-H16Q-L19V-D20L-Q22K-M23A-L85V-Q126M].

### Modulating CD25 Affinity

In some embodiments, the IL2 orthologs contain one or more mutations in positions of the IL2 sequence that either contact CD25 or alter the orientation of other positions contacting CD25 resulting in a decreased affinity for CD25. The mutations may be in or near areas known to be in close proximity to CD25 based on published crystal structures (Wang, et al (2005) Science 310:1159). IL2 residues that interact with CD25 include K35, R38, T41, F42, K43, F44, Y45, E61, E62, K64, P65, E68, V69, L72, and Y107. In some embodiments, the IL2 orthologs of the present disclosure comprise one or more of the point mutations of R38A, F41A and F42A (Suave, et al (1991) PNAS(USA)88:4636-4640); P65L (Chen et al. Cell Death and Disease (2018) 9:989); F42A/G/S/T/Q/E/N/R/K, Y45A/G/S/T/Q/E/N/D/R/K/and/or L72G/A/S/T/Q/E/N/D/R/K (Ast, et al United States Patent Application Publication 2012/0244112A1 published Sep. 27, 2012; U.S. Pat. No. 9,266,938B2 issued Feb. 23, 2016). Particular combinations of substitutions have been identified as reducing binding to CD25. In some embodiments, the IL2 orthologs of the present disclosure comprise one or more of the of the sets of substitutions [R38A-F42A-Y45A-E62A] as described in Carmenate, et al (2013) J Immunol 190:6230-6238; [F42A-Y45A-L72G] (Roche RG7461 (RO6874281); and/or [T41P-T51P] (Chang, et al (1995) Molecular Pharmacology 47:206-211). In some embodiments, the present disclosure provides hIL2 orthologs which are hIL2 polypeptides comprising one of the following sets of amino acid modifications:
[E15S-H16Q-L19V-D20L-M23A-R38A-F42A-Y45A-E62A];
[E15S-H16Q-L19V-D20L-M23A-R38A-F42A-Y45A-E62A];
[E15S-H16Q-L19V-D20L-Q22K-M23A-R38A-F42A-Y45A-E62A];
[E15S-H16Q-L19V-D20L-M23A-R38A-F42A-Y45A-E62A-Q126H];
[E15S-H16Q-L19V-D20L-M23A-R38A-F42A-Y45A-E62A-Q126H];
[E15S-H16Q-L19V-D20L-Q22K-M23A-R38A-F42A-Y45A-E62A-Q126H];
[E15S-H16Q-L19V-D20L-M23A-R38A-F42A-Y45A-E62A-Q126M]; or
[E15S-H16Q-L19V-D20L-M23A-R38A-F42A-Y45A-E62A-Q126M].

In some embodiments of the invention, the IL2 orthologs contain one or more mutations in positions of the IL2 sequence that either contact CD132 or alter the orientation of other positions contacting CD132 resulting in an altered binding to CD132. Exemplary IL2 orthologs contain one or more mutations in positions of the IL2 sequence that either contact CD132 or alter the orientation of other positions contacting CD122, resulting in an altered binding to CD132. IL2 residues believed to contact CD132 include Q11, L18, Q22, E110, N119, T123, Q126, S127, I129, S130, and T133. In some embodiments, the IL2 comprises modifications at L18 AA18 is L (wild type) or R, L, G, M, F, E, H, W, K, Q, S, V, I, Y, H, D or T; AA126 is Q (wild type) or H, M, K, C, D, E, G, I, R, S, or T; and/or AA22 is Q (wild type) or F, E, G, A, L, M, F, W, K, S, V, I, Y, H, R, N, D, T, or F.

In some embodiments, the present disclosure provides hIL2 orthologs which are hIL2 polypeptides comprising one the following sets of amino acid modifications:
[E15S-H16Q-L19V-D20L-M23A-Q126H];
[E15S-H16Q-L19V-D20L-Q22K-M23A-Q126H];
[E15S-H16Q-L19V-D20L-Q22K-Q126H];
[E15S-H16Q-L19V-D20L-M23A-Q126M];
[E15S-H16Q-L19V-D20L-Q22K-M23A-Q126M];
[E15S-H16Q-L19V-D20L-Q22K-Q126M];
[desAla1-E15S-H16Q-L19V-D20L-Q126M];
[desAla1-E15S-H16Q-L19V-D20L-Q22K-Q126M];
[desAla1-E15S-H16Q-L19V-D20L-M23A-Q126M];
[desAla1-E15S-H16Q-L19V-D20L-Q22K-M23A-Q126M];
[desAla1-E15S-H16Q-L19V-D20L-Q22K-M23A-Q126M];
[E15S-H16Q-L19V-D20L-M23A-L80F-R81D-I86V-I92F-Q126H];
[E15S-H16Q-L19V-D20L-Q22K-L80F-R81D-I86V-I92F-Q126H];
[E15S-H16Q-L19V-D20L-Q22K-M23A-L80F-R81D-I86V-I92F-Q126H];
[E15S-H16Q-L19V-D20L-M23A-L80F-R81D-I86V-I92F-Q126M];
[E15S-H16Q-L19V-D20L-Q22K-M23A-L80F-R81D-I86V-I92F-Q126M];
[E15S-H16Q-L19V-D20L-M23A-L85V-Q126H];
[E15S-H16Q-L19V-D20L-Q22K-L85V-Q126H];
[E15S-H16Q-L19V-D20L-Q22K-M23A-L85V-Q126H];
[E15S-H16Q-L19V-D20L-M23A-L85V-Q126M];
[E15S-H16Q-L19V-D20L-Q22K-L85V-Q126H]; or
[E15S-H16Q-L19V-D20L-Q22K-M23A-L85V-Q126M].

In some embodiments, the present disclosure provides hIL2 orthologs which are hIL2 polypeptides comprising one of the following sets of amino acid modifications:
[desAla1-E15S-H16Q-L19V-D20L];
[desAla1-E15S-H16Q-L19V-D20L-Q22K];
[desAla1-E15S-H16Q-L19V-D20L-Q22K-M23A];
[desAla1-E15S-H16Q-L19V-D20L-Q126H];
[desAla1-E15S-H16Q-L19V-D20L-Q22K-Q126H];
[desAla1-E15S-H16Q-L19V-D20L-Q22K-M23A-Q126H];
[desAla1-E15S-H16Q-L19V-D20L-Q126M];
[desAla1-E15S-H16Q-L19V-D20L-Q22K-Q126H];
[desAla1-E15S-H16Q-L19V-D20L-Q22K-M23A-Q126H];
[desAla1-E15S-H16Q-L19V-D20L-C125A];
[desAla1-E15S-H16Q-L19V-D20L-Q22K-C125A];
[desAla1-E15S-H16Q-L19V-D20L-Q22K-M23A-C125A];
[desAla1-E15S-H16Q-L19V-D20L-C125A-Q126H];
[desAla1-E15S-H16Q-L19V-D20L-Q22K-C125A-Q126H];
[desAla1-E15S-H16Q-L19V-D20L-Q22K-M23A-C125A-Q126H];
[desAla1-E15S-H16Q-L19V-D20L-C125A-Q126M];
[desAla1-E15S-H16Q-L19V-D20L-Q22K-C125A-Q126H];
[desAla1-E15S-H16Q-L19V-D20L-Q22K-M23A-C125A-Q126H];
[desAla1-E15S-H16Q-L19V-D20L-C125S];
[desAla1-E15S-H16Q-L19V-D20L-Q22K-C125S];
[desAla1-E15S-H16Q-L19V-D20L-Q22K-M23A-C125S];
[desAla1-E15S-H16Q-L19V-D20L-C125S-Q126H];
[desAla1-E15S-H16Q-L19V-D20L-Q22K-C125S-Q126H];
[desAla1-E15S-H16Q-L19V-D20L-Q22K-M23A-C125S-Q126H];
[desAla1-E15S-H16Q-L19V-D20L-C125S-Q126M];
[desAla1-E15S-H16Q-L19V-D20L-Q22K-C125S-Q126M]; or
[desAla1-E15S-H16Q-L19V-D20L-Q22K-M23A-C125S-Q126M].

### Conservative Amino Acid Substitutions

In addition to the foregoing modifications that contribute to the activity and selectivity of the IL2 ortholog for the CD122 orthogonal receptor, the IL2 ortholog may comprise one or more modifications to its primary structure that provide minimal effects on the activity IL2. In some embodiments, the IL2 orthologs of the present disclosure may further comprise one more conservative amino acid substitution within the wild type IL-2 amino acid sequence. Such conservative substitutions include those described by Dayhoff in The Atlas of Protein Sequence and Structure 5 (1978), and by Argos in EMBO J., 8:779-785 (1989). Conservative substitutions are generally made in accordance with the following chart depicted as Table 3.

TABLE 3
Exemplary Conservative Amino Acid Substitutions

| Wild type Residue | Substitution(s) |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu, Met, Leu, Ile |
| Phe | Met, Leu, Tyr, Trp |
| Ser | Thr |
| Thr | Ser |
| Trp | Tyr, Phe |
| Tyr | Trp, Phe |
| Val | Ile, Leu |

Substantial changes in function or immunological identity may be made by selecting amino acid substitutions that are less conservative than those indicated in Table 3. For example, substitutions may be made which more significantly affect the structure of the polypeptide backbone or disrupt secondary or tertiary elements including the substitution of an amino acid with a small uncharged side chain (e.g. glycine) with a large charge bulky side chain (asparagine). In particular, substitution of those IL2 residues which are involved in the amino acids that interact with one or more of CD25, CD122 and/or CD123 as may be discerned from the crystal structure of IL2 in association with its receptors as described in

In addition to the foregoing modifications that contribute to the activity and selectivity of the IL2 ortholog for the CD122 orthogonal receptor, the IL2 ortholog may comprise one or more modifications to its primary structure. Modifications to the primary structure as provided above may optionally further comprise modifications do not substantially diminish IL2 activity of the IL2 ortholog including but not limited to the substitutions: N30E; K32E; N33D; P34G; T37I, M39Q, F42Y, F44Y, P47G, T51L, E52K, L53N, Q57E, M104A (see U.S. Pat. No. 5,206,344).

### Removal of Glycosylation Site:

The IL2 orthologs of the present disclosure may comprises comprise modifications to eliminate the O-glycosylation site at position Thr3 of the to facilitate the production of an aglycosylated IL2 ortholog when the IL2 ortholog expressed in mammalian cells. Thus, in certain embodiments the IL2 ortholog comprise a modification which eliminates the O-glycosylation site of IL-2 at a position corresponding to residue 3 of human IL-2. In one embodiment said modification which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P which removes the glycosylation site at position 3 without eliminating biological activity (see U.S. Pat. No. 5,116,943; Weiger et al., (1989) Eur. J. Biochem., 180:295-300). In a specific embodiment, said modification is the amino acid substitution T3A. In some embodiments, the present disclosure provides hIL2 orthologs which are hIL2 polypeptides comprising one of the following sets of amino acid modifications:
[T3A-E15S-H16Q-L19V-D20L-Q22K-M23A-C125S];
[T3A-E15S-H16Q-L19V-D20L-Q22K-C125S];
[T3A-E15S-H16Q-L19V-D20L-M23A-C125S];
[T3A-E15S-H16Q-L19V-D20L-C125S];
[T3A-E15S-H16Q-L19V-D20L-Q22K-M23A-C125A];
[T3A-E15S-H16Q-L19V-D20L-M23A-C125A];
[T3A-E15S-H16Q-L19V-D20L-Q22K-C125A];
[T3A-E15S-H16Q-L19V-D20L-C125A];
[T3A-E15S-H16Q-L19V-D20L-Q22K-M23A];
[T3A-E15S-H16Q-L19V-D20L-M23A];
[T3A-E15S-H16Q-L19V-D20L-Q22K];
[T3A-E15S-H16Q-L19V-D20L];
[desAla1-T3A-E15S-H16Q-L19V-D20L-Q22K-M23A-C125S];
[desAla1-T3A-E15S-H16Q-L19V-D20L-M23A-C125S];
[desAla1-T3A-E15S-H16Q-L19V-D20L-Q22K-C125S];
[desAla1-T3A-E15S-H16Q-L19V-D20L-C125S];
[desAla1-T3A-E15S-H16Q-L19V-D20L-Q22K-M23A-C125A];
[desAla1-T3A-E15S-H16Q-L19V-D20L-M23A-C125A];
[desAla1-T3A-E15S-H16Q-L19V-D20L-Q22K-C125A];
[desAla1-T3A-E15S-H16Q-L19V-D20L-C125A];
[desAla1-T3A-E15S-H16Q-L19V-D20L-Q22K-M23A];
[desAla1-T3A-E15S-H16Q-L19V-D20L-M23A];
[desAla1-T3A-E15S-H16Q-L19V-D20L-Q22K]; or
[desAla1-T3A-E15S-H16Q-L19V-D20L].

IL2 orthologs may comprise deletion of the first two amino acids (desAla1-desPro2) as well as substitution of the Thr3 glycosylation with a cysteine residue to facilitate for selective N-terminal modification. In some embodiments, the present disclosure provides hIL2 orthologs which are hIL2 polypeptides comprising one of the following sets of amino acid modifications:
[desAla1-desPro2-T3C-E15S-H16Q-L19V-D20L-Q22K-M23A-C125S];
[desAla1-desPro2-T3C-E15S-H16Q-L19V-D20L-Q22K-C125S];
[desAla1-desPro2-T3C E15S-H16Q-L19V-D20L-M23A-C125S];
[desAla1-desPro2-T3C-E15S-H16Q-L19V-D20L-C125S];
[desAla1-desPro2-T3C-E15S-H16Q-L19V-D20L-Q22K-M23A-C125A];
[desAla1-desPro2-T3C-E15S-H16Q-L19V-D20L-Q22K-C125A];
[desAla1-desPro2-T3C E15S-H16Q-L19V-D20L-M23A-C125A];
[desAla1-desPro2-T3C-E15S-H16Q-L19V-D20L-C125A];
[desAla1-desPro2-T3C-E15S-H16Q-L19V-D20L-Q22K-M23A];
[desAla1-desPro2-T3C-E15S-H16Q-L19V-D20L-Q22K];
[desAla1-desPro2-T3C-E15S-H16Q-L19V-D20L-M23A]; or
[desAla1-desPro2-T3C-E15S-H16Q-L19V-D20L].

The IL2 orthologs may optionally further comprise a modification at position M104, in one embodiment the substitution of methionine 104 with an alanine residue (M104A) to provide a more oxidation-resistant ortholog (See Koths, et al. U.S. Pat. No. 4,752,585 issued Jun. 21, 1988).

When produced recombinantly in bacterial expression systems directly in the absence of a leader sequence, endogenous proteases result in the deletion of the N-terminal Met-Ala1 residues to provide “desAla1” IL2 orthologs. IL2 orthologs may comprise deletion of the first two amino acids (desAla1-desPro2) as well as substitution of the Thr3 glycosylation with a cysteine residue (T3C) to facilitate for N-terminal modification.

The IL2 orthologs may further comprise elimination of N-terminal amino acids at one or more of positions 1-9, alternatively positions 1-8, alternatively positions 1-7 alternatively positions 1-6, alternatively positions 1-5, alternatively positions 1-4, alternatively positions 1-3, alternatively positions 1-2. In some embodiments, the present disclosure provides hIL2 orthologs which are hIL2 polypeptides comprising one of the following sets of amino acid modifications:
[desAla1-desPro2-E15S-H16Q-L19V-D20L-Q22K-M23A];
[desAla1-desPro2-E15S-H16Q-L19V-D20L-Q22K];
[desAla1-desPro2-E15S-H16Q-L19V-D20L-M23A];
[desAla1-desPro2-E15S-H16Q-L19V-D20L];
[desAla1-desPro2-desThr3-E15S-H16Q-L19V-D20L-Q22K-M23A];
[desAla1-desPro2-desThr3-E15S-H16Q-L19V-D20L-Q22K];
[desAla1-desPro2-desThr3-E15S-H16Q-L19V-D20L-M23A];
[desAla1-desPro2-desThr3-E15S-H16Q-L19V-D20L];
[desAla1-desPro2-desThr3-desSer4-E15S-H16Q-L19V-D20L-Q22K-M23A];
[desAla1-desPro2-desThr3-desSer4-E15S-H16Q-L19V-D20L-Q22K];
[desAla1-desPro2-desThr3-desSer4-E15S-H16Q-L19V-D20L-M23A];
[desAla1-desPro2-desThr3-desSer4-E15S-H16Q-L19V-D20L];
[desAla1-desPro2-desThr3-desSer4-desSer5-E15S-H16Q-L19V-D20L-Q22K-M23A];
[desAla1-desPro2-desThr3-desSer4-desSer5-E15S-H16Q-L19V-D20L-Q22K];
[desAla1-desPro2-desThr3-desSer4-desSer5-E15S-H16Q-L19V-D20L-M23A];
[desAla1-desPro2-desThr3-desSer4-desSer5-E15S-H16Q-L19V-D20L];
[desAla1-desPro2-desThr3-desSer4-desSer5-desSer6-E15S-H16Q-L19V-D20L-Q22K-M23A];
[desAla1-desPro2-desThr3-desSer4-desSer5-desSer6-E15S-H16Q-L19V-D20L-Q22K];
[desAla1-desPro2-desThr3-desSer4-desSer5-desSer6-E15S-H16Q-L19V-D20L-M23A]; or
[desAla1-desPro2-desThr3-desSer4-desSer5-desSer6-E15S-H16Q-L19V-D20L].

In some embodiments of the disclosure, the IL2 ortholog comprises amino acid substitutions to avoid vascular leak syndrome, a substantial negative and dose limiting side effect of the use of IL2 therapy in human beings without out substantial loss of efficacy. See, Epstein, et al., U.S. Pat. No. 7,514,073B2 issued Apr. 7, 2009. Examples of such modifications which are included in the 112 orthologs of the present disclosure include one or more of R38W, R38G, R39L, R39V, F42K, and H55Y.

In some embodiments, IL2 orthologs may be affinity matured to enhance their activity with respect to the hoCD122 ECD. An “affinity matured” polypeptide is one having one or more alteration(s) in one or more residues which results in an improvement in the affinity of the orthogonal polypeptide for the cognate orthogonal receptor, or vice versa, compared to a parent polypeptide which does not possess those alteration(s). Affinity maturation can be done to increase the binding affinity of the IL2 ortholog by at least about 10%, alternatively at least about 50%, alternatively at least about 100% alternatively at least about 150%, or from 1 to 5-fold as compared to the “parent” polypeptide. An engineered IL2 ortholog of the present invention activates its cognate orthogonal receptor, as discussed above, but has significantly reduced binding and activation of the wild-type IL2 receptor when assessed by ELISA and/or FACS analysis using sufficient amounts of the molecules under suitable assay conditions.

As discussed above, the compositions of the present disclosure include IL2 orthologs that have been modified to provide for an extended lifetime in vivo and/or extended duration of action in a subject. Such modifications to provided extended lifetime and/or duration of action include modifications to the primary sequence of the IL2 ortholog, expression as fusion proteins with carrier molecules, (e.g. HSA, albumin, acylation), and Fc fusions. In some embodiments, the IL2 ortholog may comprise certain amino acid substitutions that result in prolonged in vivo lifetime. In some embodiments, the IL2 orthologs of the present disclosure comprise one, two or all three of the substitutions V91R, K97E and T113N which are reporte to enhance lifetime in vivo. See, e.g. Dakshinamurthi, et al. (International Journal of Bioinformatics Research (2009) 1(2):4-13).

In some embodiments, the IL2 ortholog is expressed as a fusion protein with an albumin molecule (e.g. human serum albumin) which is known in the art to facilitate extended exposure in vivo. In one embodiment of the invention, the hIL2 analog is conjugated to albumin referred to herein as an “IL2 ortholog albumin fusion.” The term “albumin” as used in the context hIL2 analog albumin fusions include albumins such as human serum albumin (HSA), cyno serum albumin, and bovine serum albumin (BSA). In some embodiments, the HSA the HSA comprises a C34S or K573P amino acid substitution relative to the wild type HSA sequence According to the present disclosure, albumin can be conjugated to a hIL2 ortholog at the carboxyl terminus, the amino terminus, both the carboxyl and amino termini, and internally (see, e.g., U.S. Pat. Nos. 5,876,969 and 7,056,701). In the HSA-hIL2 ortholog polypeptide conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a hIL2 analog polypeptide fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker such as a peptide linker or modified version thereof as more fully discussed below.

Alternatively, the hIL2 analog albumin fusion comprises IL2 orthologs that are fusion proteins which comprise an albumin binding domain (ABD) polypeptide sequence and an IL2 ortholog polypeptide. As alluded to above, fusion proteins which comprise an albumin binding domain (ABD) polypeptide sequence and an hIL2 analog polypeptide can, for example, be achieved by genetic manipulation, such that the nucleic acid coding for HSA, or a fragment thereof, is joined to the nucleic acid coding for the one or more IL2 ortholog sequences. In some embodiments, the albumin-binding peptide comprises the amino acid sequence DICLPRWGCLW (SEQ ID NO:28).

The IL2 ortholog polypeptide can also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria, dendritic cells, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen Such conjugated forms, if desired, can be used to produce antibodies against a polypeptide of the present disclosure.

In some embodiments, the IL2 ortholog is conjugated (either chemically or as a fusion protein) with an XTEN which provides extended duration of comparable to PEGylation. XTEN polymers suitable for use in conjunction with the IL2 orthologs of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193 Jun. 13, 2016. The XTEN polymer may fusion protein may incorporate a protease sensitive cleavage site between the XTEN polypeptide and the IL2 ortholog such as an MMP-2 cleavage site.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

In some embodiments, the IL-2 mutein also may be linked to additional polypeptide therapeutic agents including therapeutic compounds such as anti-inflammatory compounds or antineoplastic agents, therapeutic antibodies (e.g. Herceptin), immune checkpoint modulators, immune checkpoint inhibitors (e.g. anti-PD1 antibodies), cancer, cytokines such as CSF, GSF, GMCSF, TNF, erythropoietin, immunomodulators or cytokines such as the interferons or interleukins, a neuropeptide, reproductive hormones such as HGH, FSH, or LH, thyroid hormone

The polypeptide fusion proteins may comprise directly linkage between the domains or via a linker molecule. Suitable polypeptide linkers include “flexible linkers” which are generally of sufficient length to permit some movement between the modified polypeptide sequences and the linked components and molecules. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers include glycine polymers (G)ₙ, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)ₙ, glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein.

In some embodiments, the nucleic acids encoding the orthogonal IL2 ligand and orthogonal CD122 receptor may be provided on separate vectors. In one embodiment, to co-express orthogonal IL-2 and orthogonal IL-2Rb via a dual vector system, each protein sequence is cloned into a different vector (e.g. lentiviral, retroviral, transposon, etc.) with or without a selectable marker (e.g. puromycin, fluorescent marker, truncated LNGFR, etc.). Alternatively, the nucleic acids encoding the orthogonal IL2 ligand and orthogonal CD122 receptor may be co-expressed from single vector. To co-express orthogonal IL2 ligand and orthogonal CD122 receptor IL-2 and orthogonal IL-2Rb via a single vector, the sequences of the two proteins are separated by a cleavage peptide (e.g. T2A, P2A, E2A, etc.) or an internal ribosomal entry site (IRES) with or without a selectable marker. If using a cleavage peptide, no stop codon is included in the position 1 sequence. The two proteins are cloned into either position 1 or position 2:

5′—“position 1”_cleavage peptide/IRES_“position 2”-3′

Immune cells are transduced, transfected, or electroporated with these constructs in their transmissible form (e.g. lentivirus, retrovirus, mRNA, etc.). Examples of cleavage peptide IRES sequences which may be incorporated into a vector include but are not limited to a P2A sequence such as GSGATNFSLLKQAGDVEENPGP (SEQ ID NO: 29), a T2A sequence such as GSGEGRGSLLTCGDVEENPGPx (SEQ ID NO: 38), or an E2A sequence such as GSGQCTNYALLKLAGDVESNPGP (SEQ ID NO: 39).

### Membrane-Bound Linkers:

To create a membrane-bound orthogonal I1-2 ligand, the sequence of membrane-bound linkers, the sequence of the orthogonal 112 ligand is modified to incorporate an anchor protein such as the glycosylphosphatidylinositol (GPI) anchors provided in Table 4 below or the transmembrane domain of cell surface molecules (e.g. CD8, CD28, CD40L, etc.) are cloned into the C-terminal end of the orthogonal-IL2 and expressed as a single polypeptide. Additional linkers such as G4S (SEQ ID NO: 40), whitlow, etc. can be inserted in between the membrane-bound linkers and the orthogonal E1-2 to achieve greater flexibility and alterations in orthogonal IL-2/orthogonal IL-2RB activity.

TABLE 4
Carboxy-terminal glycosylphosphatidylinositiol
(GPI) anchors

| Protein | Species | GPI signal sequence* |
|---|---|---|
| Alkaline | H. | TACDLAPPAGTTDAAHPGR |
| phosphatase | sapiens | SVVPALLPLLAGTLLLLET |
| | | ATAP (SEQ ID NO: 41) |
| Decay | H. | HETTPNKGSGTTSGTTRLL |
| accelerating | sapiens | SGHTCFTLTGLLGTLVTMG |
| factor | | LLT (SEQ ID NO: 42) |
| PARP | T. brucei | EPEPEPEPEPEPGAATLKS |
| | | VALPFAIAAAALVAAF |
| | | (SEQ ID NO: 43) |
| Prion protein | hamster | QKESQAYYDGRRSSAVLFS |
| | | SPPVILLISFLIFLMVG |
| | | (SEQ ID NO: 44) |
| Thy-1 | rat | KTINVIRDKLVKCGGISLL |
| | | VQNTSWLLLLLLSLSFLQ |
| | | ATDFISI |
| | | (SEQ ID NO: 45) |
| Varient surface | T. brucei | ESNCKWENNACKDSSILVT |
| glycoprotein | | KKFALTVVSAAFVALLF |
| | | (SEQ ID NO: 46) |
| Acetyl | T. | NQFLPKLLNATACSSSGTS |
| cholinesterase | marmorata | SSKGIIFYVLFSILYLIFY |
| | | (SEQ ID NO: 47) |

*The underlined amino acid is site of attachment of the GPI.

See, e.g., Essentials of Glycobiology [Internet]. 3′d edition Varki A, Cummings R D, Esko J D, et al., editors Cold Spring Harbor (N.Y.): Cold Spring Harbor Laboratory Press; 2015-2017

Carboxy-terminal fusion of the transmembrane domains of cell surface proteins can include: CD80, CD86, CD8, CD28, 4-1bb, and other cell surface proteins.

In some embodiments, the ortho IL-2Rb is joined to the orthogonal IL2 ligand, optionally via a linker. In one embodiment, the orthogonal IL-2/orthogonal IL-2Rb fusion, a linker is introduced between the orthogonal IL-2 C-terminus and the orthogonal IL-2Rb N-terminus without its membrane signal peptide. This linker can include any portion of IL2Ra extracellular domain and an additional linker such as G4S (SEQ ID NO: 40), whitlow, myc, etc. A particular example of such a fusion protein of the structure H2N-orthogonal orthogonal IL-2-IL2Ra extracellular domain fragment-linker-orthogonal IL-2Rb (no membrane signal peptide)-COOH is provided by

(SEQ ID NO: 30)
ELCDDDPPEIPHATFKAMAYKEGTMLNCECK
RGFRRIKSGSLYMLCTGNSSHSSWDNQCQCT
SSATRNTTKQVTPQPEEQKERKTTEMQSPMQ
PVDQASLPGHCREPPPWENEATERIYHFVVG
QMVYYQCVQGYRALHRGPAESVCKMTHGKTR
WTQPQLICTGEMETSQFPGEEKPQASPEGRP
ESETSCLVTTTDFQIQTEMAATMETSIFTTE
YQ

The underlined sequence denotes fragment of IL-2Ra that is directly involved in the interaction between IL-2, IL-2Ra, and IL-2Rb. See, e.g., WO2017201432A3.

### Fc Fusions

In some embodiments, the IL2 fusion protein may incorporate an Fc region derived from the IgG subclass of antibodies that lacks the IgG heavy chain variable region. The “Fc region” can be a naturally occurring or synthetic polypeptide that is homologous to the IgG C-terminal domain produced by digestion of IgG with papain. IgG Fc has a molecular weight of approximately 50 kDa. The mutant IL-2 polypeptides can include the entire Fc region, or a smaller portion that retains the ability to extend the circulating half-life of a chimeric polypeptide of which it is a part. In addition, full-length or fragmented Fc regions can be variants of the wild type molecule. That is, they can contain mutations that may or may not affect the function of the polypeptides; as described further below, native activity is not necessary or desired in all cases. In certain embodiments, the IL-2 mutein fusion protein (e.g., an IL-2 partial agonist or antagonist as described herein) includes an IgGl, IgG2, IgG3, or IgG4 Fc region. Exemplary Fc regions can include a mutation that inhibits complement fixation and Fc receptor binding, or it may be lytic, i.e., able to bind complement or to lyse cells via another mechanism such as antibody-dependent complement lysis (ADCC).

In some embodiments, the IL2 ortholog comprises a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The IL2 orthologs may provide the entire Fc region, or a smaller portion that retains the ability to extend the circulating half-life of a chimeric polypeptide of which it is a part. In addition, full-length or fragmented Fc regions can be variants of the wild type molecule. In a typical presentation, each monomer of the dimeric Fc carries a heterologous polypeptide, the heterologous polypeptides being the same or different.

In some embodiments, when the IL2 ortholog is to be expressed in the format of an Fc fusion, particularly in those situations when the polypeptide chains conjugated to each subunit of the Fc dimer are different, the nucleic acid sequences encoding the Fc fusion may be engineered to generate an ligand possessing a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g. tyrosine or tryptophan) creating a projection from the surface (“knob”) and ii) in the CH3 domain of a second heavy chain, an amino acid residue is replaced with an amino acid residue having a smaller side chain (e.g. alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g. an IL2 ortholog) on a first Fc monomer with a “knob” modification and a second polypeptide on the second Fc monomer possessing a “hole” modification to facilitate the expression of heterodimeric polypeptide conjugates.

The Fc region can be “lytic” or “non-lytic,” but is typically non-lytic. A non-lytic Fc region typically lacks a high affinity Fc receptor binding site and a C1q binding site. The high affinity Fc receptor binding site of murine IgG Fc includes the Leu residue at position 235 of IgG Fc. Thus, the Fc receptor binding site can be inhibited by mutating or deleting Leu 235. For example, substitution of Glu for Leu 235 inhibits the ability of the Fc region to bind the high affinity Fc receptor. The murine C1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fc receptor binding site and a C1q binding site. The high affinity Fc receptor binding site includes the Leu residue at position 235 of IgG Fc, and the C1q binding site includes the Glu 318, Lys 320, and Lys 322 residues of IgG 1. Lytic IgG Fc has wild type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2: 125, 1994).

In certain embodiments, the amino- or carboxyl-terminus of an IL2 ortholog of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

In some embodiments, the Fc domain monomer comprises at least one mutation relative to a wild-type human IgG1, IgG2, or IgG4 Fc region as described in U.S. Pat. No. 10,259,859B2, the entire teaching of which is herein incorporated by reference.

In other embodiments, the IL2 ortholog can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the IL2 ortholog polypeptide further comprises a C-terminal c-myc epitope tag.

In some embodiment, the IL2 orthologs (including fusion proteins of such IL2 orthologs) of the present invention are expressed as a fusion protein with one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present invention are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present invention are peptides comprising 3-6 contiguous histidine residues (SEQ ID NO: 48) such as a six-histidine peptide (His)₆ (SEQ ID NO: 49) and are frequently referred to in the art as “His-tags.”

In some embodiments, the IL2 ortholog is provided as a fusion protein with a polypeptide sequence (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the IL2 ortholog sequence and the sequence of the targeting domain of the fusion protein.

In some embodiments, the targeting domain of the IL2 ortholog fusion protein specifically binds to a cell surface molecule of a tumor cell. In one embodiment wherein the ECD of the CAR of a CAR-T cell specifically binds to CD-19, the IL2 ortholog may be provided as a fusion protein with a CD-19 targeting moiety thereby providing selective activation of the engineered cells expressing the orthogonal receptor ECD in the environment of the tumor. For example, in one embodiment wherein the ECD of the CAR of an CAR-T cell is an scFv molecule that provides specific binding to CD-19, the IL2 ortholog is provided as a fusion protein with a CD-19 targeting moiety such as a single chain antibody (e.g., an scFv or VHH) that specifically binds to CD-19.

In some embodiments, the fusion protein comprises an IL-2 mutein and the anti-CD19 sdFv FMC63 (Nicholson, et al. (1997) Mol Immunol 34: 1157-1165). Similarly, in some embodiments wherein the ECD of the CAR of an CAR-T cell specifically binds to BCMA, the IL2 ortholog is provided as a fusion protein with a BCMA targeting moiety, such as antibody comprising the CDRs of anti-BMCA antibodies as described in in Kalled, et al. (U.S. Pat. No. 9,034,324 issued May 9, 2015) or antibodies comprising the CDRs as described in Brogdon, et al., (U.S. Pat. No. 10,174,095 issued Jan. 8, 2019). In some embodiments the IL2 ortholog is provided as a fusion protein with a GD2 targeting moiety, such as an antibody comprising the CDRs of described in Cheung, et al., (U.S. Pat. No. 9,315,585 issued Apr. 19, 2016) or the CDRs derived from ME36.1 (Thurin et al., (1987) Cancer Research 47:1229-1233), 14G2a, 3F8 (Cheung, et al., 1985 Cancer Research 45:2642-2649), hu14.18, 8B6, 2E12, or ic9.

In an alternative embodiment, the targeted IL2 orthologs of the present disclosure may be administered in combination with CAR-T cell therapy to provide targeted delivery of the IL2 ortholog to the CAR-T cell based on an extracellular receptor of the CAR-T cell such as by and anti-FMC63 antibody to target the IL2 activity to the CAR-T cells and rejuvenate exhausted CAR-T cells in vivo. Consequently, embodiments of the present disclosure include targeted delivery of IL2 orthologs by conjugation of such IL2 orthologs to antibodies or ligands that are designed to interact with specific cell surface molecules of CAR-T cells. An example of such a molecule would an anti-FMC63-hIL2 ortholog.

In other embodiments, the chimeric polypeptide includes the mutant IL-2 polypeptide and a heterologous polypeptide that functions to enhance expression or direct cellular localization of the mutant IL-2 polypeptide, such as the Aga2p agglutinin subunit (see, e.g., Boder and Wittrup, Nature Biotechnol. 15:553-7, 1997).

### Protein Transduction Domain Fusion Proteins:

In some embodiments, the IL2 ortholog further comprises a “Protein Transduction Domain” or “PTD.” A PTD is a polypeptide, polynucleotide, carbohydrate, or organic or inorganic molecule that facilitates traversing a lipid bilayer, micelle, cell membrane, organelle membrane, or vesicle membrane. The incorporation of a PTD into an IL2 ortholog facilitates the molecule traversing a membrane. In some embodiments, a PTD is covalently linked to the amino or carboxy terminus of an IL2 ortholog. In some embodiments, the PTD is incorporated as part of an PTD-IL2 ortholog fusion protein, either at the N or C terminus of the molecule.

Exemplary protein transduction domains include, but are not limited to, a minimal decapeptide protein transduction domain (corresponding to residues 47-57 of HIV-1 TAT); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008), Transportan (as described in Wierzbicki, et al., (2014) Folio Histomchemica et Cytobiologica 52(4): 270-280 and Pooga, et a (1998) FASEB J 12(1)67-77 and commercially available from AnaSpec as Catalog No. AS-61256); KALA (as described in Wyman et al., (1997) Biochemistry 36(10) 3008-3017 and commercially available from AnaSpec as Catalog No. AS-65459); Antennapedia Peptide (as described in Pietersz et al., (2001) Vaccine 19:1397 and commercially available from AnaSpec as Catalog No. AS-61032); TAT 47-57 (commercially available from AnaSpec as Catalog No. AS-60023).

In some embodiments, the IL-2 conjugate comprises a plasma half-life in a human subject of greater than 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 10 days, 14 days, or 30 days.

### Construction of Nucleic Acid Sequences Encoding the IL2 Ortholog

The present disclosure provides nucleic acid sequences and recombinant vectors encoding IL2 orthologs of FORMULA #2, recombinant receptors comprising the orthogonal ECD of CD122 of FORMULA #1, as well as CARs comprising a variety of targeting domains, targeted against a variety of tumor antigens IL2 ortholog, CD122 or CAR The nucleic acid sequence encoding the desired IL2 ortholog can be synthesized by chemical means using an oligonucleotide synthesizer. A variety of commercial vendors will prepare nucleic acid sequences to order based on a particular

The nucleic acid molecules are not limited to sequences that encode polypeptides; some or all of the non-coding sequences that lie upstream or downstream from a coding sequence (e.g., the coding sequence of IL2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules of the present disclosure may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IL2 ortholog, CD122 or CAR may be obtained from various commercial sources that provide custom made nucleic acid sequences. Amino acid sequence variants of the these polypeptides to the produce the nucleic acid sequences of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding the IL2 orthologs and expressing those sequences in a suitably transformed host include, but are not limited to, using a PCR-assisted mutagenesis technique. Mutations that consist of deletions or additions of amino acid residues to an IL2 polypeptide can also be made with standard recombinant techniques. In the event of a deletion or addition, the nucleic acid molecule encoding IL2 is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

An IL2 ortholog of the present disclosure may be produced recombinantly not only directly, but also as a fusion polypeptide with a heterologous polypeptide, e.g. a signal sequence or other polypeptide having a specific cleavage site at the N-terminus or C-terminus of the mature IL2 ortholog. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In some embodiments, the signal sequence is the signal sequence that is natively associated with the IL2 ortholog (i.e. the human IL2 signal sequence). The inclusion of a signal sequence depends on whether it is desired to secrete the IL2 ortholog from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild type IL2 signal sequence be used. Alternatively, heterologous mammalian signal sequences may be suitable, such as signal sequences from secreted polypeptides of the same or related species, as well as viral secretory leaders, for example, the herpes simplex gD signal. When the recombinant host cell is a yeast cell such as Saccharomyces cerevisiae, the alpha mating factor secretion signal sequence may be employed to achieve extracellular secretion of the IL2 ortholog into the culture medium as described in Singh, U.S. Pat. No. 7,198,919 B1 issued Apr. 3, 2007.

In the event the IL2 ortholog to is to be expressed as a chimera (e.g., a fusion protein comprising an IL2 ortholog and a heterologous polypeptide sequence), the chimeric protein can be encoded by a hybrid nucleic acid molecule comprising a first sequence that encodes all or part of the IL2 ortholog and a second sequence that encodes all or part of the heterologous polypeptide. For example, subject IL2 orthologs described herein may be fused to a hexa-histidine tag (SEQ ID NO: 49) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the IL2 ortholog. For example, the N-terminus may be linked to a targeting domain and the C-terminus linked to a hexa-histidine tag (SEQ ID NO: 49) purification handle.

The complete amino acid sequence of the polypeptide (or fusion/chimera) to be expressed can be used to construct a back-translated gene. A DNA oligomer containing a nucleotide sequence coding for IL2 ortholog can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the recombinant protein (IL2 ortholog, orthogonal CD122, or CAR) may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

The ABD of the CAR may be monovalent or multivalent and comprise one or multiple (e.g. 1, 2, or 3) polypeptide sequence (e.g. scFv, VHH, ligand) that specifically bind to a cell surface tumor antigen. In some embodiments, tumor antigens and CARs comprising ABDs that selectively bind to such cell surface tumor are known in the art (see, e.g., Dotti, et al., Immunol Rev. 2014 January; 257(1). The methods and compositions of the present disclosure are useful in conjunction with CAR therapy wherein the ABD of the CAR specifically binds a tumor antigen including but not limited to CD123, CD19, CD20, BCMA, CD22, CD30, CD70, Lewis Y, GD3, GD3, mesothelin, ROR CD44, CD171, EGP2, EphA2, ErbB2, ErbB3/4, FAP, FAR IL11Ra, PSCA, PSMA and NCAM. Antibodies reactive with these targets are well known in the literature and one of skill in the art is capable of isolating the CDRs from such antibodies for the construction of polypeptide sequences of single chain antibodies (e.g. scFvs, CDR grafted VHHs and the like) that may be incorporated into the ABD of the CAR.

### STAT3

The present disclosure provides an orthogonal CD122 comprising, in addition to a native STAT5 recognition motif, one or more STAT3 binding motifs. The additional STAT3 binding motifs boosts the signaling and also stabilizes the IL2 response.

STAT proteins act as transcriptional activators upon phosphorylation of a conserved tyrosine residue at the C terminus followed by translocation into the nucleus, where they bind to DNA and activate target gene transcription. Hennighausen and Robinson (2008) Genes Dev. 2008; 22:711-21. Seven STAT proteins have been identified in the STAT family: STAT1, STAT2, STAT3, STAT4, STAT5A, STAT5B and STAT6, and they have functions in a variety of pathways, from innate and acquired immunity to cell proliferation, differentiation and survival. Basham et al., (2008) Nucleic Acids Res. 2008 June; 36(11): 3802-3818.

STATs binding motifs are typically present in cytokine receptors and binding of their respective cytokine ligand activates the tyrosine kinases in the Janus kinase (JAK) families, which phosphorylate certain tyrosine residues in the intracellular domains. The phosphorylated receptor recruits STATs to STAT recognition motifs on the receptor and the STAT becomes phosphorylated. The phosphorylated STATs dimerize and translocate to the nucleus wherein they initiate transcription of a variety of genes. Hennighausen, supra.

Of these STAT proteins, STAT5 is activated by the binding of cytokines including IL2, IL-4, IL-7, IL-9, IL-15, and IL21 to their cognate receptors. Lara E. Kallal & Christine A. Biron (2013) Changing partners at the dance, JAK-STAT, 2:1, e23504, DOI: 10.4161/jkst.23504, Page 2, Col. 2. For example, CD122 and orthogonal CD122 contain a STAT5 recognition motif and can recruit and activate STAT5. Activated STAT5 results in the activation of transcription of genes such as Cis, spi2.1, and Socs-1. Basham et al., Nucleic Acids Res supra.

The STAT5 binding motif has a sequence of YX₁X₂L (SEQ ID NO: 33). X₁ and X₂ can be any natural amino acid. In some cases, X₁ and X₂ are the same amino acid residues. In some cases, X₁ and X₂ are different amino acid residues. In one embodiment, the STAT5 motif has a sequence of YLSL (SEQ ID NO: 34).

The STAT3 binding motif is not present in the naturally occurring form (wild-type) human CD122. It is typically present in other cytokine receptors that bind to IL-6, IL-10, IL21, IFNalpha, IFNbeta, IFNgamma, and IFN a2b Upon activation, STAT3 targets Bcl-XL, survivin, cyclin D1, and activating c-myc. Kallal, et al, supra. STAT3 can be activated through tyrosine phosphorylation by a variety of cytokines whose receptors share the gp130 chain, including IL-6 and IL21, oncostatin M (OSM) and leukemia inhibitory factor (LIF). STAT3 has roles in a variety of biological functions including oncogenesis, angiogenesis and tumor metastasis, and, anti-apoptosis. See e.g. Sun et al. (2006) FEBS Lett. 580(25):5880-4 and Fukada, et al. (1996) Immunity 5(5): 449-460. The incorporation of one or more functional STAT3 signaling motifs in the intracellular domain of the orthogonal CD122 upregulates anti-apoptotic factors in the modified cells expressing the orthogonal IL2 in response to binding of a cognate IL2 ortholog to the ECD of such STAT3 modified orthogonal CD122. Consequently, orthogonal cells which express an orthogonal CD122 comprising an intracellular domain incorporating one or more functional STAT3 domains have an enhanced survival and longer life and therefore a longer duration of action in vivo.

In some embodiments, the present disclosure provides a human orthogonal CD122 (comprising the intact STAT5 motif) has been modified to introduce one or more STAT3 binding motifs and the modified human CD122 so produced retains STAT5 recognition motif and gains one or more STAT3 binding motifs.

In some embodiments, the modified orthogonal CD122 may comprise one, two, three, or more STAT3 binding motifs. the STAT3 recognition motif has an amino acid sequence of YX₁X₂Q (SEQ ID NO: 35). In some embodiments, X₁ is selected from the group consisting of L, R, F, M, and X₂ is selected from the group consisting of R, K, H, and P. In some embodiments, the STAT3 recognition motif has an amino acid sequence selected from the group consisting of: YLRQ (SEQ ID NO: 11); YLKQ (SEQ ID NO: 12); YRHQ (SEQ ID NO: 13); YLRQ (SEQ ID NO: 14); YFKQ (SEQ ID NO: 15); YLPQ (SEQ ID NO: 16); YMPQ (SEQ ID NO: 17), and YDKPH (SEQ ID NO: 18).

In some embodiments, the one or more STAT3 binding motifs may be incorporated at the C-terminus of the orthogonal CD122 ICD or as an internal sequence of the ICD of the orthogonal CD122

In some cases, the modified human CD122 comprises one or more STAT3 binding motifs fused to the C-terminus of the intracellular domain of a human CD122. In some representative embodiments, the orthogonal CD122 comprises the addition of C-terminal STAT3 recognition domains resulting in orthogonal CD122 polypeptides of the structures:

(SEQ ID NO: 50)
Ortho-CD122-GGYLRQ;
(SEQ ID NO: 51)
Ortho-CD122-GGYLKQ;
(SEQ ID NO: 52)
Ortho-CD122-GGYRHQ;
(SEQ ID NO: 50)
Ortho-CD122-GGYLRQ;
(SEQ ID NO: 53)
Ortho-CD122-GGYFKQ;
(SEQ ID NO: 54)
Ortho-CD122-GGYLPQ;
(SEQ ID NO: 55)
Ortho-CD122-GGYMPQ;
and
(SEQ ID NO: 56)
Ortho-CD122-GGYDKPH.

In some cases, the orthogonal CD122 comprises a STAT3 binding motif that is connected to the human CD122 through a linker. Linkers can be derived from naturally-occurring proteins or synthetic sequences. Methods for designing linkers are well-known in the art, for example, as disclosed in Chen et al. (2013) Adv. Drug. Deliv. Rev. 65(10):1357-1369, the relevant portion thereof is herein incorporated by reference. Suitable linkers can be readily selected and can be of any suitable length, such as 1 amino acid (e.g., Gly), 2, 3, 4, 5, 6, 7, 8, 9, 10, 10-20, or 20-30 amino acids. Examples of flexible linkers include glycine polymers (G)ₙ, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)ₙ, glycine-alanine polymers, alanine-serine polymers, glycine-serine polymers. Glycine and glycine-serine polymers are relatively unstructured, and therefore may serve as a neutral tether between components. The modified orthogonal CD122 may comprise one or more STAT3 binding motifs and one or more linker sequences. Said linker sequences may connect the human CD122 and one of the STAT3 binding motifs or connect individual STAT3 binding motifs. The one or more linker sequence may have the same or different sequences.

In some embodiments, one or more STAT3 binding motifs are present as an internal (i.e., at neither C nor N terminus) sequence of the orthogonal CD122. A modified orthogonal CD122 of this configuration can be produced by identifying a suitable region within the orthogonal CD122 ICD amino acid sequence that can be mutated to create a STAT3 binding motif. In one embodiment, the naturally occurring ICD of the CD122 (which is typically present in the orthogonal CD122) possess sequences similar to the STAT3 binding motif that may readily be modified to create a STAT3 binding motif with minimal modification. For example a region comprising a four-nucleotide sequence that begins with a tyrosine residue. One such region in the native human CD122 encodes a sequence of YFTYDPYSEE (SEQ ID NO: 57), which is located between positions 355 and position 364 of the native human CD122 protein. In some embodiments, one or two of the YFTY (SEQ ID NO: 58), YDPY (SEQ ID NO: 59), or YSEE (SEQ ID NO: 60) comprised in this region are substituted with a STAT3 recognition motif to produce a modified human CD122 disclosed herein.

Such modified orthogonal CD122s are able to induce STAT3 and STAT5 signaling upon binding to a cognate IL2 ligand and the ability can be confirmed by e.g., monitoring the levels of phosphorylated STAT3 and STAT5 in response to contacting a cell expressing the orthogonal CD122 having the STAT modified ICD with a cognate IL2 ortholog. For example, the modified human CD122 can be introduced and expressed in T cells and antibodies that are specific to phospho-STAT5 and phosphor-STAT3 are used to detect the phosphorylation of STAT3 and STAT5. One exemplary method for detecting a recombinant protein's ability to induce STAT3 and STAT5 signaling is described in Kagoya et al. (2018) Nat Med. 24(3):352-359.

In some embodiments, this disclosure provides a method of stimulating an engineered cell expressing a modified human orthogonal CD122 comprising one or more STAT3 binding motifs, the method comprising contacting the engineered cell with a human IL2 ortholog which is a cognate ligand of the modified human orthogonal CD122 thereby stimulating the engineered cells. In some embodiments, this disclosure provides a method of increasing the intracellular levels of STAT3 and STAT5 in an engineered cell expressing a modified human orthogonal CD122 comprising one or more STAT3 binding motifs, the method comprising contacting the engineered cell with a human IL2 ortholog which is a cognate ligand of the modified human orthogonal CD122 such that the intracellular levels of STAT3 and STAT5 are increased in the engineered cell.

In some embodiments, this disclosure provides a method of stimulating an engineered T cell expressing a modified human orthogonal CD122 comprising one or more STAT3 binding motifs, the method comprising contacting the engineered T cell with a human IL2 ortholog which is a cognate ligand of the modified human orthogonal CD122 thereby stimulating the engineered T cells. In some embodiments, this disclosure provides a method of increasing the intracellular levels of STAT3 and STAT5 in an engineered T cell expressing a modified human orthogonal CD122 comprising one or more STAT3 binding motifs, the method comprising contacting the engineered T cell with a human IL2 ortholog which is a cognate ligand of the modified human orthogonal CD122 such that the intracellular levels of STAT3 and STAT5 are increased in said cell.

In some embodiments, this disclosure provides a method of stimulating an CAR-T cell expressing a modified human orthogonal CD122 comprising one or more STAT3 binding motifs, the method comprising contacting the CAR-T cell with a human IL2 ortholog which is a cognate ligand of the modified human orthogonal CD122 thereby stimulating the engineered T cells. In some embodiments, this disclosure provides a method of increasing the intracellular levels of STAT3 and STAT5 in an CAR-T cell expressing a modified human orthogonal CD122 comprising one or more STAT3 binding motifs, the method comprising contacting the CAR-T cell with a human IL2 ortholog which is a cognate ligand of the modified human orthogonal CD122 such that the intracellular levels of STAT3 and STAT5 are increased in said CAR-T cell.

In some embodiments, this disclosure provides a method to selectively activate an orthogonal cell in a mixed cell population comprising orthogonal cells and naturally occurring cells, the method comprising contacting the mixed population of cells with IL2 ortholog that is a cognate ligand for the orthogonal CD122 of the orthogonal cell, thereby selectively activating the orthogonal cells. In some embodiments, this disclosure provides a method to selectively activate an orthogonal cell in a mixed cell population, the cell population comprising orthogonal cells and native cells, the method comprising contacting the mixed population of cells with a orthogonal human IL2, which specifically bind to the modified orthogonal human CD122 of the hoCD122 cells, thereby selectively activating the engineered immune cells.

### Engineered Cells:

The preparation of the engineered immune cells of the present invention is achieved by transforming isolated immune cell with an expression vector comprising a nucleic acid sequence encoding a receptor comprising an orthogonal CD122 ECD of FORMULA #1 and the hIL2 orthogonal ligand of FORMULA #2. The IL2 orthologs of the present invention may be employed in methods of selectively expanding such engineered T cells (e.g., human T-cells) which have been engineered to express a corresponding orthogonal CD122 receptor. T-cells useful for engineering with the constructs described herein include naïve T-cells, central memory T-cells, effector memory T-cells or combination thereof. T cells for engineering as described above are collected from a subject or a donor may be separated from a mixture of cells by techniques that enrich for desired cells or may be engineered and cultured without separation. Alternatively, the T cells for engineering may be separated from other cells. Techniques providing accurate separation include fluorescence activated cell sorters. The cells may be selected against dead cells by employing dyes associated with dead cells (e.g., propidium iodide). The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification or may be frozen at liquid nitrogen temperatures and stored, being thawed and capable of being reused. The cells will usually be stored in 10% DMSO, 50% FCS, 40% RPMI 1640 medium.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Seminars in Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

### CAR-T Cells

In one embodiment of the invention the T-cell expressing an orthogonal CD122 ECD of FORMULA #1 and the hIL2 orthogonal ligand of FORMULA #2 is a T-cell (e.g., human T-cell) which has been modified to surface express a chimeric antigen receptor (a ‘CAR-T’ cell).

As used herein, the term antigen binding domain (ABD) refers to a polypeptide that contains at least one binding domain that specifically binds to at least one antigen expressed on the surface of a target cell. In some embodiments, the ABD comprises a polypeptide with two binding domains that selectively bind to the same antigen or two different antigens on the surface of the target cells. The ABD may be any polypeptide that specifically binds to one or more antigens expressed on the surface of a target cell. The ABD is a polypeptide that

CARs can further comprise a transmembrane domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. For example, the transmembrane polypeptide can be a subunit of the T-cell receptor such as α, β, γ or ζ, polypeptide constituting CD3 complex, CD25, CD122 or CD132 chain, subunit chain of Fc receptors, in particular Fcγ receptor III. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1) In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g Pace, et al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3ζ, CD4, CD8, CD28, etc.

The transmembrane domain of the CAR can further comprise a hinge region between said extracellular ligand-binding domain and said transmembrane domain. The term “hinge region” is typically used in the art to refer to any polypeptide that functions to link the transmembrane domain to the extracellular ligand-binding domain. Hinge domains are typically included to provide more flexibility and accessibility for the binding domain of the CAR to interact with the cell surface of the target cell. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. Examples of suitable hinge domains useful in the context of the present disclosure include but are not limited to CD8 alpha chain, FcγRIIIα receptor or IgG1 respectively.

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

In some embodiments, the CAR may also provide a co-stimulatory domain. The term “co-stimulatory domain”, refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεR1γ or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3 ζ signal. Coincidental delivery of the delivered co-stimulatory signal enhances cytokine secretion and antitumor activity induced by CAR-transduced T-cells. The co-stimulatory domain is usually be membrane proximal relative to the CD3ζ domain. Third-generation CARs include a tripartite signaling domain, comprising for example a CD28, CD3ζ, OX40 or 4-1BB signaling region. In fourth generation, or “armored car” CAR T-cells are further modified to express or block molecules and/or receptors to enhance immune activity such as the expression of IL-12, IL-18, IL-7, and/or IL-10; 4-1BB ligand, CD-40 ligand.

Examples of intracellular signaling domains comprising may be incorporated into the CAR of the present invention include (amino to carboxy): CD3ζ; CD28-41BB-CD3ζ; CD28-OX40-CD3ζ; CD28-41BB-CD3ζ; 41BB-CD-28-CD3ζ and 41BB-CD3ζ.

The term CAR includes CAR variants including but not limited split CARs, ON-switch CARS, bispecific or tandem CARs, inhibitory CARs (iCARs) and induced pluripotent stem (iPS) CAR-T cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The term “bispecific or tandem CARs” refers to CARs which include a secondary CAR binding domain that can either amplify or inhibit the activity of a primary CAR.

The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. T cells with specificity for both tumor and off-target tissues can be restricted to tumor only by using an antigen-specific iCAR introduced into the T cells to protect the off-target tissue (Fedorov, et al., (2013). Science Translational Medicine, 5:215) Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility. In some embodiments, the iCAR comprises an single chain antibody (e.g. scFv, VHH, etc) that specifically binds to an inhibitory antigen, one or more intracellular derived from the ICDs immunoinhibitory receptors (including but not limited to CTLA-4, PD-1, LAG-3, 2B4 (CD244), BTLA (CD272), KIR, TIM-3, TGFbeta receptor dominant negative analog etc.) via a transmembrane region that inhibits T cell function specifically upon antigen recognition.

The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor T-cells (CAR-T cells) are T-cells which have been recombinantly modified by transduction with an expression vector encoding a CAR in substantial accordance with the teaching above.

In some embodiments, an engineered T cell is allogeneic with respect to the individual that is treated. Graham et al. (2018) Cell 7(10) E155. In some embodiments an allogeneic engineered T cell is fully HLA matched. However not all patients have a fully matched donor and a cellular product suitable for all patients independent of HLA type provides an alternative.

Because the cell product may consist of a subject's own T-cells, the population of the cells to be administered is to the subject is necessarily variable. Consequently identifying the optimal concentration of the Additionally, since the CAR-T cell agent is variable, the response to such agents can vary and thus involves the ongoing monitoring and management of therapy related toxicities which are managed with a course of pharmacologic immunosuppression or B cell depletion prior to the administration of the CAR-T cell treatment. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection. The engineered cells may be infused to the subject in any physiologically acceptable medium by any convenient route of administration, normally intravascularly, although they may also be introduced by other routes, where the cells may find an appropriate site for growth

If the T cells used in the practice of the present invention are allogeneic T cells, such cells may be modified to reduce graft versus host disease. For example, the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-CreI homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD3ζ as a TCR inhibitory molecule.

Examples of CAR architectures useful in the practice of the present invention include but are not limited to the following examples which illustrate the ECD targeting domain(s) and the architecture of the ICD of the CAR:
anti-CD19-CD3ζ
anti-CD19-CD28-41BB-CD3ζ,
anti-CD19-CD28-CD3ζ
anti-CD19-CD28-OX40-CD3ζ
anti-CD19-CD28-41BB-CD3ζ
anti-CD19-OX40-CD3ζ
anti-CD19-OX40-CD28-CD3ζ
anti-CD19-41BB-CD3ζ
anti-CD19-ICOS-CD3ζ
anti-CD19-ICOS-41BB-CD3ζ
anti-CD19-41BB-ICOS-CD3ζ
anti-CD19-41BB-OX40-CD3ζ,
anti-CD19-41BB-CD28-CD3ζ.
anti-PSMA-CD3ζ
anti-PSMA-CD28-41BB-CD3ζ,
anti-PSMA-CD28-CD3ζ
anti-PSMA-CD28-OX40-CD3ζ
anti-PSMA-CD28-41BB-CD3ζ
anti-PSMA-OX40-CD3ζ
anti-PSMA-OX40-CD28-CD3ζ
anti-PSMA-41BB-CD3ζ
anti-PSMA-ICOS-CD3ζ
anti-PSMA-ICOS-41BB-CD3ζ
anti-PSMA-41BB-ICOS-CD3ζ
anti-PSMA-41BB-OX40-CD3ζ,
anti-PSMA-41BB-CD28-CD3ζ.
anti-BCMA-CD3ζ
anti-BCMA-CD28-41BB-CD3ζ,
anti-BCMA-CD28-CD3ζ
anti-BCMA-CD28-OX40-CD3ζ
anti-BCMA-CD28-41BB-CD3ζ
anti-BCMA-OX40-CD3ζ
anti-BCMA-OX40-CD28-CD3ζ
anti-BCMA-41BB-CD3ζ
anti-BCMA-ICOS-CD3ζ
anti-BCMA-ICOS-41BB-CD3ζ
anti-BCMA-41BB-ICOS-CD3ζ
anti-BCMA-41BB-OX40-CD3ζ,
anti-BCMA-41BB-CD28-CD3ζ.
anti-mesothelin-CD3ζ
anti-mesothelin-CD28-41BB-CD3ζ,
anti-mesothelin-CD28-CD3ζ
anti-mesothelin-CD28-OX40-CD3ζ
anti-mesothelin-CD28-41BB-CD3ζ
anti-mesothelin-OX40-CD3ζ
anti-mesothelin-OX40-CD28-CD3ζ
anti-mesothelin-41BB-CD3ζ
anti-mesothelin-ICOS-CD3ζ
anti-mesothelin-ICOS-41BB-CD3ζ
anti-mesothelin-41BB-ICOS-CD3ζ
anti-mesothelin-41BB-OX40-CD3ζ,
anti-mesothelin-41BB-CD28-CD3ζ.
[anti-CD19 & anti-CD20]-CD3ζ
[anti-CD19 & anti-CD20]-CD28-41BB-CD3ζ,
[anti-CD19 & anti-CD20]-CD28-CD3ζ
[anti-CD19 & anti-CD20]-CD28-OX40-CD3ζ
[anti-CD19 & anti-CD20]-CD28-41BB-CD3ζ
[anti-CD19 & anti-CD20]-OX40-CD3ζ
[[anti-CD19 & anti-CD20]-OX40-CD28-CD3ζ
[anti-CD19 & anti-CD20]-41BB-CD3ζ
[anti-CD19 & anti-CD20]-ICOS-CD3ζ
[anti-CD19 & anti-CD20]-ICOS-41BB-CD3ζ
[anti-CD19 & anti-CD20]-41BB-ICOS-CD3ζ
[anti-CD19 & anti-CD20]-41BB-OX40-CD3ζ,
[anti-CD19 & anti-CD20]-41BB-CD28-CD3ζ.
anti-EGFR-CD3ζ
anti-EGFR-CD28-41BB-CD3ζ,
anti-EGFR-CD28-CD3ζ
anti-EGFR-CD28-OX40-CD3ζ
anti-EGFR-CD28-41BB-CD3ζ
anti-EGFR-OX40-CD3ζ
anti-EGFR-OX40-CD28-CD3ζ
anti-EGFR-41BB-CD3ζ
anti-EGFR-ICOS-CD3ζ
anti-EGFR-ICOS-41BB-CD3ζ
anti-EGFR-41BB-ICOS-CD3ζ
anti-EGFR-41BB-OX40-CD3ζ, and
anti-EGFR-41BB-CD28-CD3ζ.
[anti-CD19 & anti-CD22]-CD3ζ
[anti-CD19 & anti-CD22]-CD28-41BB-CD3ζ,
[anti-CD19 & anti-CD22]-CD28-CD3ζ
[anti-CD19 & anti-CD22]-CD28-OX40-CD3ζ
[anti-CD19 & anti-CD22]-CD28-41BB-CD3ζ
[anti-CD19 & anti-CD22]-OX40-CD3ζ
[anti-CD19 & anti-CD22]-OX40-CD28-CD3ζ
[anti-CD19 & anti-CD22]-41BB-CD3ζ
[anti-CD19 & anti-CD22]-ICOS-CD3ζ
[anti-CD19 & anti-CD22]-ICOS-41BB-CD3ζ
[anti-CD19 & anti-CD22]-41BB-ICOS-CD3ζ
[anti-CD19 & anti-CD22]-41BB-OX40-CD3ζ,
[anti-CD19 & anti-CD22]-41BB-CD28-CD3ζ.

The designation, for example: [anti-CD19 & anti-CD20] is meant to illustrate the situation where the ECD targeting moiety of the CAR is bispecific, in this case for CD19 and CD20.

In some embodiments, the present disclosure provides therapeutic methods to the treatment of a subject suffering from a disease, disorder or condition, the method comprising the administration to said subject a population cells comprising orthogonal cells (e.g. orthogonal human immune cells) in combination with the administration of an IL2 ortholog that is a cognate ligand for the orthogonal CD122 expressed on said orthogonal cells.

In some embodiments, the present disclosure provides therapeutic methods for the treatment of a subject suffering from a disease, disorder or condition, the method comprising the administration to said subject a population cells, the population of cells comprising orthogonal human immune cells in combination with a therapeutically effective amount of a human IL2 ortholog that is a cognate ligand for the orthogonal human CD122 expressed on the orthogonal human immune cells.

In some embodiments, the present disclosure provides therapeutic methods for the treatment of a subject suffering from a neoplastic disease, disorder or condition, the method comprising the administered to the subject a population cells, the population of cells comprising human orthogonal TILs (hoTILs) in combination with a therapeutically effective amount of a human IL2 ortholog that is a cognate ligand for the orthogonal human CD122 expressed on said hoTILs.

In some embodiments, the present disclosure provides therapeutic methods for the treatment of a subject suffering from a neoplastic disease, disorder or condition, the method comprising the administered to the subject a population cells, the population of cells comprising human orthogonal CAR-T (hoCAR-T) cells in combination with a therapeutically effective amount of a human IL2 ortholog that is a cognate ligand for the orthogonal human CD122 expressed on said hoTILs. In some embodiments, the hoCAR-T cells of the method is selected from the group consisting of CD19 hoCAR-T cells, CD20, hoCAR-T cells, BCMA hoCAR-T cells,

In some embodiments, the present disclosure provides therapeutic methods for the treatment of a subject suffering from a neoplastic disease, disorder or condition, the method comprising the administration to said subject a population cells, the population of cells comprising human orthogonal CAR-T (hoCAR-T) cells, in combination with the administration of a human IL2 ortholog that is a cognate ligand for the orthogonal human CD122 expressed on said hoTILs.

In some cases, the subject is suffering from a neoplastic disease and the orthogonal human immune cells are CD8+ T cells. In some cases, the subject is suffering from an autoimmune disease the orthogonal human immune cells are Treg cells. In some cases, the engineered immune cells are hoCAR-T cells

In another aspect, the invention features a cytotoxic cell, e.g., a naturally or non-naturally occurring T cell, NK cell or cytotoxic T cell or cell of an NK cell line, e.g., NK92, comprising (a) a first KIR-CAR described herein. In one embodiment, said cytotoxic cell is T cell. In one embodiment, said cytotoxic cell is an NK cell. In one embodiment, said cytotoxic cell is from an NK cell line, e.g., an NK92 cell.

The present disclosure provides a method of preparing a population of cells enriched for orthogonal human immune cells, the method comprising: (a) obtaining a sample of a tissue from said subject; (b) isolating a population of cells comprising immune cells from the tissue; (c) contacting the population of cells with recombinant vector encoding an orthogonal CD122; (d) culturing said cell population in the presence of an effective concentration of an IL2 ortholog that is a cognate ligand for the orthogonal CD122 encoded by the vector.

The present disclosure provides a method of preparing a population of cells enriched for orthogonal human immune cells, the method comprising: (a) obtaining a sample of a tissue from said subject; (b) isolating a population of cells comprising immune cells from the tissue; (c) contacting the population of cells with recombinant vector encoding an orthogonal CD122; (d) culturing said cell population in the presence of an effective concentration of an IL2 ortholog that is a cognate ligand for the orthogonal CD122 encoded by the vector.

Combination of Engineered Immune with Supplementary Therapeutic Agents:

The present disclosure provides the for the use of the engineered immune cell of the present disclosure in combination with one or more additional active agents (“supplementary agents”). Such further combinations are referred to interchangeably as “supplementary combinations” or “supplementary combination therapy” and those therapeutic agents that are used in combination with engineered immune cell of the present disclosure are referred to as “supplementary agents.” As used herein, the term “supplementary agents” includes agents that can be administered or introduced separately, for example, formulated separately for separate administration (e.g., as may be provided in a kit) and/or therapies that can be administered or introduced in combination with the engineered immune cell.

### Chemotherapeutic Agents:

In some embodiments, the supplementary agent is a chemotherapeutic agent. In some embodiments the supplementary agent is a “cocktail” of multiple chemotherapeutic agents. IN some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivatives such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent is one or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the IL2 ortholog is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

Combination with Therapeutic Antibodies

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and tri-specific killer engager (TriKE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Le^{y}, VEGF (e.g. bevacizumab), VEGFR, VEGFR2 (e.g. ramucirumab), PDGFRα (e.g. olartumumab), EGFR (e.g. cetuximab, panitumumab and necitumumab), ERBB2 (e.g. trastuzumab), ERBB3, MET, IGF1R, EPHA3, TRAIL R1, TRAIL R2, RANKL RAP, tenascin, integrin αVβ3, and integrin α4β1.

Examples of antibody therapeutics which are FDA approved and may be used as supplementary agents for use in the treatment of neoplastic disease indicated on include those provided in Table 5 below.

TABLE 5
FDA Antineoplastic Disease Antibodies and Indications

| Name | Tradename(s) | Target; format | Indication |
|---|---|---|---|
| [fam]-trastuzumab | Enhertu | HER2; Humanized IgG1 ADC | HER2+ breast cancer |
| deruxtecan | | | |
| Enfortumab vedotin | Padcev | Nectin-4; Human IgG1 ADC | Urothelial cancer |
| Polatuzumab | Polivy | CD79b; Humanized IgG1 | Diffuse large B-cell lymphoma |
| vedotin | | ADC | |
| Cemiplimab | Libtayo | PD-1; Human mAb | Cutaneous squamous cell |
| | | | carcinoma |
| Moxetumomab | Lumoxiti | CD22; Murine IgG1 dsFv | Hairy cell leukemia |
| pasudotox | | immunotoxin | |
| Mogamuizumab | Poteligeo | CCR4; Humanized IgG1 | Cutaneous T cell lymphoma |
| Tildrakizumab | Ilumya | IL-23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, ADC | Hematological malignancy |
| ozogamicin | | | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFa; Human IgG1 | Soft tissue sarcoma |
| Ixekizumab | Taltz | IL-17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Nivolumab | Opdivo | PD1; Human IgG4 | Melanoma, non-small cell lung |
| | | | cancer |
| Blinatumomab | Blincyto | CD19, CD3; Murine bispecific | Acute lymphoblastic leukemia |
| | | tandem scFv | |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL-6; Chimeric IgG1 | Castleman disease |
| Obinutuzumab | Gazyva | CD20; Humanized IgG1; | Chronic lymphocytic leukemia |
| | | Glycoengineered | |
| Ado-trastuzumab | Kadcyla | HER2; Humanized IgG1, ADC | Breast cancer |
| emtansine | | | |
| Pertuzumab | Perjeta | HER2; Humanized IgG1 | Breast Cancer |
| Brentuximab | Adcetris | CD30; Chimeric IgG1, ADC | Hodgkin lymphoma, systemic |
| vedotin | | | anaplastic large cell lymphoma |
| Ipilimumab | Yervoy | CTLA-4; Human IgG1 | Metastatic melanoma |
| Ofatumumab | Arzerra | CD20; Human IgG1 | Chronic lymphocytic leukemia |
| Certolizumab pegol | Cimzia | TNF; Humanized Fab, | Crohn disease |
| | | pegylated | |
| Catumaxomab | Removab | EPCAM/CD3; Rat/mouse | Malignant ascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab-I131 | Bexxar | CD20; Murine IgG2a | Non-Hodgkin lymphoma |
| Ibritumomab | Zevalin | CD20; Murine IgG1 | Non-Hodgkin lymphoma |
| tiuxetan | | | |
| Gemtuzumab | Mylotarg | CD33; Humanized IgG4, ADC | Acute myeloid leukemia |
| ozogamicin | | | |
| Trastuzumab | Herceptin | HER2; Humanized IgG1 | Breast cancer |
| Infliximab | Remicade | TNF; Chimeric IgG1 | Crohn disease |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

In some embodiments, where the antibody is a bispecific antibody targeting a first and second tumor antigen such as HER2 and HER3 (abbreviated HER2×HER3), FAP×DR-5 bispecific antibodies, CEA×CD3 bispecific antibodies, CD20×CD3 bispecific antibodies, EGFR-EDV-miR16 trispecific antibodies, gp100×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PD1×CTLA-4 bispecific antibodies, CD123×CD3 bispecific antibodies, gpA33×CD3 bispecific antibodies, B7-H3×CD3 bispecific antibodies, LAG-3×PD1 bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies,

CD20×CD3 bispecific antibodies, CD123×CD3 bispecific antibodies, SSTR2×CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies.

Such therapeutic antibodies may be further conjugated to one or more chemotherapeutic agents (e.g antibody drug conjugates or ADCs) directly or through a linker, especially acid, base or enzymatically labile linkers.

Combination with Physical Methods:

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising an IL2 ortholog and one or more supplementary agents. In some embodiments, the present disclosure further contemplates the use of an IL2 ortholog in combination with surgery (e.g. tumor resection). In some embodiments, the present disclosure further contemplates the use of an IL2 ortholog in combination with bone marrow transplantation, peripheral blood stem cell transplantation or other types of transplantation therapy.

Combination with Immune Checkpoint Modulators:

In some embodiments, a “supplementary agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

An immune checkpoint whose activation results in inhibition or downregulation of the immune response is referred to herein as a “negative immune checkpoint pathway modulator.” The inhibition of the immune response resulting from the activation of a negative immune checkpoint modulator diminishes the ability of the host immune system to recognize foreign antigen such as a tumor-associated antigen. The term negative immune checkpoint pathway includes, but is not limited to, biological pathways modulated by the binding of PD1 to PDL1, PD1 to PDL2, and CTLA4 to CDCD80/86. Examples of such negative immune checkpoint antagonists include but are not limited to antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152).

In one embodiment, an immune checkpoint pathway the activation of which results in stimulation of the immune response is referred to herein as a “positive immune checkpoint pathway modulator.” The term positive immune checkpoint pathway modulator includes, but is not limited to, biological pathways modulated by the binding of ICOSL to ICOS(CD278), B7-H6 to NKp30, CD155 to CD96, OX40L to OX40, CD70 to CD27, CD40 to CD40L, and GITRL to GITR. Molecules which agonize positive immune checkpoints (such natural or synthetic ligands for a component of the binding pair that stimulates the immune response) are useful to upregulate the immune response. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”).

The immune response mediated by immune checkpoint pathways is not limited to T-cell mediated immune response. For example, the KIR receptors of NK cells modulate the immune response to tumor cells mediated by NK cells. Tumor cells express a molecule called HLA-C, which inhibits the KIR receptors of NK cells leading to a dimunition or the anti-tumor immune response. The administration of an agent that antagonizes the binding of HLA-C to the KIR receptor such an anti-KIR3 mab (e.g. lirilumab, BMS) inhibits the ability of HLA-C to bind the NK cell inhibitory receptor (KIR) thereby restoring the ability of NK cells to detect and attack cancer cells. Thus, the immune response mediated by the binding of HLA-C to the KIR receptor is an example a negative immune checkpoint pathway the inhibition of which results in the activation of a of non-T-cell mediated immune response.

In one embodiment, the immune checkpoint pathway modulator is a negative immune checkpoint pathway inhibitor/antagonist. In another embodiment, immune checkpoint pathway modulator employed in combination with the IL2 ortholog is a positive immune checkpoint pathway agonist. In another embodiment, immune checkpoint pathway modulator employed in combination with the IL2 ortholog is an immune checkpoint pathway antagonist.

The term “negative immune checkpoint pathway inhibitor” refers to an immune checkpoint pathway modulator that interferes with the activation of a negative immune checkpoint pathway resulting in the upregulation or enhancement of the immune response. Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton N.J.), pembrolizumab (Keytruda® MK-3475, lambrolizumab, commercially available from Merck and Company, Kenilworth N.J.), and atezolizumab (Tecentriq®, Genentech/Roche, South San Francisco Calif.). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

The term PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline. Aptamer compounds are also described in the literature useful as PD1 pathway inhibitors (Wang, et al. (2018) 145:125-130).

The term PD1 pathway inhibitors includes peptidyl PD1 pathway inhibitors such as those described in Sasikumar, et al., U.S. Pat. No. 9,422,339 issued Aug. 23, 2016, and Sasilkumar, et al., U.S. Pat. No. 8,907,053 issued Dec. 9, 2014. CA-170 (AUPM-170, Aurigene/Curis) is reportedly an orally bioavailable small molecule targeting the immune checkpoints PDL1 and VISTA. Pottayil Sasikumar, et al. Oral immune checkpoint antagonists targeting PD-L1/VISTA or PD-L1/Tim3 for cancer therapy. [abstract]. In: Proceedings of the 107th Annual Meeting of the American Association for Cancer Research; 2016 Apr. 16-20; New Orleans, La. Philadelphia (Pa.): AACR; Cancer Res 2016; 76(14 Suppl): Abstract No. 4861. CA-327 (AUPM-327, Aurigene/Curis) is reportedly an orally available, small molecule that inhibit the immune checkpoints, Programmed Death Ligand-1 (PDL1) and T-cell immunoglobulin and mucin domain containing protein-3 (TIM3).

The term PD1 pathway inhibitors includes small molecule PD1 pathway inhibitors. Examples of small molecule PD1 pathway inhibitors useful in the practice of the present invention are described in the art including Sasikumar, et al., 1,2,4-oxadiazole and thiadiazole compounds as immunomodulators (PCT/IB2016/051266 filed Mar. 7, 2016, published as WO2016142833A1 Sep. 15, 2016) and Sasikumar, et al. 3-substituted-1,2,4-oxadiazole and thiadiazole PCT/IB2016/051343 filed Mar. 9, 2016 and published as WO2016142886A2), BMS-1166 and Chupak LS and Zheng X. Compounds useful as immunomodulators. Bristol-Myers Squibb Co. (2015) WO 2015/034820 A1, EP3041822 B1 granted Aug. 9, 2017; WO2015034820 A1; and Chupak, et al. Compounds useful as immunomodulators. Bristol-Myers Squibb Co. (2015) WO 2015/160641 A2. WO 2015/160641 A2, Chupak, et al. Compounds useful as immunomodulators. Bristol-Myers Squibb Co. Sharpe, et al. Modulators of immunoinhibitory receptor PD-1, and methods of use thereof, WO 2011082400 A2 published Jul. 7, 2011; U.S. Pat. No. 7,488,802 (Wyeth) issued Feb. 10, 2009;

In some embodiments, combination of engineered immune cell and one or more PD1 immune checkpoint modulators are useful in the treatment of neoplastic conditions for which PD1 pathway inhibitors have demonstrated clinical effect in human beings either through FDA approval for treatment of the disease or the demonstration of clinical efficacy in clinical trials including but not limited to melanoma, non-small cell lung cancer, small cell lung cancer, head and neck cancer, renal cell cancer, bladder cancer, ovarian cancer, uterine endometrial cancer, uterine cervical cancer, uterine sarcoma, gastric cancer, esophageal cancer, DNA mismatch repair deficient colon cancer, DNA mismatch repair deficient endometrial cancer, hepatocellular carcinoma, breast cancer, Merkel cell carcinoma, thyroid cancer, Hodgkins lymphoma, follicular lymphoma, diffuse large B-cell lymphoma, mycosisfungoides, peripheral T-cell lymphoma. In some embodiments, the combination of engineered immune cell and an PD1 immune checkpoint modulator is useful in the treatment of tumors characterized by high levels of expression of PDL1, where the tumor has a tumor mutational burden, where there are high levels of CD8+ T-cell in the tumor, an immune activation signature associated with IFNγ and the lack of metastatic disease particularly liver metastasis. In some embodiments, the engineered T-cell is modified to delete the PDL1 receptor (See, e.g. Rupp, et al (2017) CRISPR/Cas9-mediated PD-1 disruption enhances anti-tumor efficacy of human chimeric antigen receptor T cells Science Reports 7:737

In some embodiments, the engineered immune cell of the present disclosure in combination with an antagonist of a negative immune checkpoint pathway that inhibits the binding of CTLA4 to CD28 (“CTLA4 pathway inhibitor”). Examples of CTLA4 pathway inhibitors are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (Medarex, Inc.) issued Oct. 20, 2009)

In some embodiments, engineered immune cell of the present disclosure is administered in combination with an antagonist of a negative immune checkpoint pathway that inhibits the binding of BTLA to HVEM (“BTLA pathway inhibitor”). A number of approaches targeting the BTLA/HVEM pathway using anti-BTLA antibodies and antagonistic HVEM-Ig have been evaluated, and such approaches have suggested promising utility in a number of diseases, disorders and conditions, including transplantation, infection, tumor, and autoimmune disease (See e.g. Wu, et al., (2012) Int. J. Biol. Sci. 8:1420-30).

In some embodiments, the engineered immune cell of the present disclosure is administered in combination with an antagonist of a negative immune checkpoint pathway that inhibits the ability TIM3 to binding to TIM3-activating ligands (“TIM3 pathway inhibitor”). Examples of TIM3 pathway inhibitors are known in the art and with representative non-limiting examples described in International Patent Application No. PCT/US2016/021005 published Sep. 15, 2016; Lifke, et al. United States Patent Publication No. US 20160257749 A1 published Sep. 8, 2016 (F. Hoffian-LaRoche), Karunsky, U.S. Pat. No. 9,631,026 issued Apr. 27, 2017; Karunsky, Sabatos-Peyton, et al. U.S. Pat. No. 8,841,418 issued Sep. 23, 2014; U.S. Pat. No. 9,605,070; Takayanagi, et al., U.S. Pat. No. 8,552,156 issued Oct. 8, 2013.

In some embodiments, the engineered immune cell of the present disclosure is administered in combination with an inhibitor of both LAG3 and PD1 as the blockade of LAG3 and PD1 has been suggested to synergistically reverse anergy among tumor-specific CD8+ T-cells and virus-specific CD8+ T-cells in the setting of chronic infection. IMP321 (ImmuFact) is being evaluated in melanoma, breast cancer, and renal cell carcinoma. See generally Woo et al., (2012) Cancer Res 72:917-27; Goldberg et al., (2011) Curr. Top. Microbiol. Immunol. 344:269-78; Pardoll (2012) Nature Rev. Cancer 12:252-64; Grosso et al., (2007) J. Clin. Invest. 117:3383-392].

In some embodiments, the engineered immune cell of the present disclosure is administered in combination with an A2aR inhibitor. A2aR inhibits T-cell responses by stimulating CD4+ T-cells towards developing into TReg cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the 112 orthologs for use in the treatment disorders such as cancer and Parkinson's disease.

In some embodiments, the engineered immune cell of the present disclosure is administered in combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g. cancer) in a mammalian subject by the administration of engineered immune cell of the present disclosure in combination with an agent(s) that modulate at least one immune checkpoint pathway including immune checkpoint pathway modulators that modulate two, three or more immune checkpoint pathways.

In some embodiments the engineered immune cell of the present disclosure is administered in combination with an immune checkpoint modulator that is capable of modulating multiple immune checkpoint pathways. Multiple immune checkpoint pathways may be modulated by the administration of multi-functional molecules which are capable of acting as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of hIL2 orthologs in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the engineered immune cell of the present disclosure may be administered in combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a IL2 ortholog of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

### Chemokine and Cytokine Agents as Supplementary Agents:

In some embodiments the engineered immune cell of the present disclosure is administered in combination with additional cytokines including but not limited to IL-7, IL-12, IL-15 and IL-18 including analogs and variants of each thereof.

### Activation-Induced Cell Death Inhibitors

In some embodiments the engineered immune cell of the present disclosure is administered in combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the engineered immune cell of the present disclosure described herein include but are not limited to cyclosporin A (Shih, et al., (1989) Nature 339:625-626, IL-16 and analogs (including rhIL-16, Idziorek, et al., (1998) Clinical and Experimental Immunology 112:84-91), TGFb1 (Genesteir, et al., (1999) J Exp Med 189(2): 231-239), and vitamin E (Li-Weber, et al., (2002) J Clin Investigation 110(5):681-690).

### Physical Methods

In some embodiments, the supplementary agent is a anti-neoplastic physical methods including but not limited to radiotherapy, cryotherapy, hyperthermic therapy, surgery, laser ablation, and proton therapy.

### Lymphodepletion

In some embodiments, the methods of the present disclosure optionally include the step of lymphodepletion prior to the administration of the orthogonal cells to the subject. Lymphodepletion is typically performed in a subject in conjunction with adoptive cell therapy by the administration of a mixed cell population comprising the CAR-Ts or TILs in combination with the administration of non-specific agents (e.g. IL2) to support the CAR-Ts or TILs. Studies suggest that lymphodepletion may have therapeutic benefits in the context of adoptive cell transfer. It is reported that lymphodepletion depletes Tregs, removes cellular “sinks”, provided physical space for the adoptively transferred cells to proliferate in the subject, reduces the competition for homeostatic cytokines such as IL-7 and IL-15 and reduces immunosuppressive lymphoid and myeloid populations. However, it should be noted that lymphodepletion is associated with certain serious toxicities associated with adoptive cell transfer treatment. Lymphodepleting regimens cause a short, but deep lymphopenia and neutropenia, with full bone marrow recovery within 7-10 days, typically not requiring hematopoietic stem cell support. In those circumstance where lymphodepletion is deemed necessary by the healthcare professional, the subject should be closely monitored to address any resulting toxicities.

In those circumstances where lymphodepletion is employed, lymphodepletion may be achieved by treating said subject with a lymphodepleting treatment regimen comprising anti-CD52 antibodies, purine analogs, and the like. In some embodiments, the lymphodepleting treatment regimen is a lymphodepleting non-myeloablative chemotherapeutic regimen (NMA chemotherapy). One example of a lymphodepleting non-myeloablative chemotherapeutic regimen (NMA chemotherapy) commonly used in clinical practice comprises the following steps: approximately of 2 days intravenous administration of cyclophosphamide to the subject at a dose of approximately 60 mg/kg followed by 5 days fludarabine administration at a dose of approximately 25 mg/m². In some instances, the lymphodepleting treatment regimen optionally or further comprises exposing the subject to total body ionizing irradiation (TBI) at a dose of from about 1 gray to about 80 gray, optionally from about 1 gray to about 20 gray, optionally from about 2 gray to about 15 gray. Murine models had shown that response rates upon TIL therapy improved after prior lymphodepletion by total body irradiation (TBI). The amount of radiation applied varies depending on the type and stage of cancer being treated. Higher doses of radiation are typically administered in the case of solid epithelial tumors where lower doses may be sufficient for non-solid tumors such as lymphomas, and as part of a maintenance protocol from about 0.5 gray to about 4 gray, preferably about 1-2 gray.

The compositions and methods of the present disclosure also provide a method for the treatment of a subject with a population of cells, the population of cells comprising orthogonal cells (e.g. orthogonal human immune cells, hoCAR-T cells, hoTILs, hoNK cells) in the absence of lymphodepletion. The methods and compositions of the present disclosure typically obviate the for lymphodepletion of the subject in adoptive cell therapy by both (or either) providing a substantially purified population of engineered cells largely devoid of contamination by non-engineered cells when the foregoing ex vivo method is employed and/or the selective activation and expansion of the orthogonal cells with an IL2 ortholog of the present invention which provide substantially reduced off-target effects of non-specific proliferative agents such as IL2.

In one aspect of the invention, the lymphodepletion currently employed in association with CAR-T therapy may be obviated or reduced by the use of engineered immune cell of the present disclosure of. As noted above, the lymphodepletion is commonly employed to enable expansion of the CAR-T cells. However, the lymphodepletion is also associated with major side effects of CAR-T cell therapy. Because the engineered immune cell of the present disclosure enables the selective activation and expansion of the human immune cell (e.g. hoTIL or hoCAR-T) in the mixed population, the cell product administered is substantially enriched for the therapeutically effective orthogonal human immune cell (e.g. hoTIL or hoCAR-T) such the need for lymphodepletion prior to administration of the cell product comprising the engineered immune cell of the present disclosure is avoided or substantially reduced. The compositions and method of the present invention enable the practice of adoptive cell therapy without or with reduced lymphodepletion prior to administration of the adoptive cell product to the subject.

In some embodiments, the present disclosure provides a method of treating a mammalian subject suffering from a disease, disorder or condition amenable to treatment with adoptive cell therapy, the method comprising administering to said subject a population cells comprising a therapeutically effective amount of engineered immune cell of the present disclosure in the absence of prior lymphodepletion. In one embodiment, the present disclosure provides a method of treating a human subject suffering from a neoplastic disease, disorder or condition with TIL adoptive cell therapy the method comprising administering to said subject a population cells comprising a therapeutically effective amount of hoTILs in the absence of prior lymphodepletion. In one embodiment, the present disclosure provides a method of treating a human subject suffering from a neoplastic disease, disorder or condition with CAR-T adoptive cell therapy, the method comprising administering to said subject a population cells comprising a therapeutically effective amount of hoCAR-T cells in the absence of prior lymphodepletion.

### Pharmaceutical Formulations:

The engineered immune cell of the present disclosure of the present invention may be administered to a subject in a pharmaceutically acceptable dosage form. The preferred formulation depends on the intended mode of administration and therapeutic application. Pharmaceutical dosage forms of engineered immune cell of the present disclosure comprise physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, polymeric amino acids, amino acid copolymers, and lipid aggregates (such as oil droplets or liposomes).

The compositions may also comprise pharmaceutically-acceptable, non-toxic carriers, excipients, stabilizers or diluents, which are defined as vehicles commonly used to formulate pharmaceutical compositions for animal or human administration. The diluent is selected so as not to affect the biological activity of the combination. Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN M, PLURONICS' or polyethylene glycol (PEG).

Formulations to be used for in vivo administration are typically sterile. Sterilization of the compositions of the present invention may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997.

Where the contacting is performed in vivo, an effective dose of engineered cells, including without limitation CAR-T cells modified to express an orthogonal CD122 receptor, are infused to the recipient, Dosage and frequency may vary depending on the agent; mode of administration; nature of the IL2 ortholog, and the like. It will be understood by one of skill in the art that such guidelines will be adjusted for the individual circumstances. The dosage may also be varied for route of administration, e.g. intramuscular, intraperitoneal, intradermal, subcutaneous, intravenous infusion and the like. Generally at least about 10⁴ engineered cells/kg are administered, at least about 10⁵ engineered cells/kg; at least about 10⁶ engineered cells/kg, at least about 10⁷ engineered cells/kg, or more.

Where the engineered immune cells of the present disclosure are T cells, an enhanced immune response may be manifest as an increase in the cytolytic response of T cells towards the target cells present in the recipient, e.g. towards elimination of tumor cells, infected cells; decrease in symptoms of autoimmune disease; and the like. In some embodiments when the engineered T cell population is to be administered to a subject, the subject is provided with immunosuppressive course of therapy prior to or in combination with the administration of the engineered T cell population. Examples of such immunosuppressive regimens include but are not limited to systemic corticosteroids (e.g., methylprednisolone). Therapies for B cell depletion include intravenous immunoglobulin (IVIG) by established clinical dosing guidelines to restore normal levels of serum immunoglobulin levels. In some embodiments, prior to administration of the CAR-T cell therapy of the present invention, the subject may optionally be subjected to a lymphodepleting regimen. One example of a such lymphodepleting regimen consists of the administration to the subject of fludarabine (30 mg/m²intravenous daily for 4 days) and cyclophosphamide (500 mg/m² IV daily for 2 days starting with the first dose of fludarabine).

Engineered immune cells of the present disclosure be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The engineered immune cells of the present disclosure can be administered by any suitable means, usually parenteral. Parenteral infusions include intramuscular, intravenous (bolus or slow infusion), intraarterial, intraperitoneal, intrathecal or subcutaneous administration. In the typical practice, the engineered T cells are infused to the subject in a physiologically acceptable medium, normally intravascularly, although they may also be introduced into any other convenient site, where the cells may find an appropriate site for growth. Usually, at least 1×10⁵ cells/kg will be administered, at least 1×10⁶ cells/kg, at least 1×10⁷ cells/kg, at least 1×10⁸ cells/kg, at least 1×10⁹ cells/kg, or more, usually being limited by the number of T cells that are obtained during collection.

For example, typical ranges for the administration engineered immune cells of the present disclosure cells for use in the practice of the present invention range from about 1×10⁵ to 5×10⁸viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses OF engineered immune cells of the present disclosure over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

In one embodiment, the present disclosure provides a method of treating a subject suffering from a disease, disorder or condition amendable to treatment with CAR-T cell therapy (e.g. cancer) by the administration of a orthogonal ligand expressing CAR-Ts in the absence of lymphodepletion prior to administration of the orthogonal ligand CAR-Ts. In one embodiment, the present disclosure provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T-cells from the individual; (b) enriching the biological sample for the presence of T-cells; (c) transfecting the T-cells with one or more expression vectors comprising a nucleic acid sequence encoding a CAR and a nucleic acid sequence encoding an orthogonal CD122 receptor, the antigen targeting domain of the CAR being capable of binding to at least one antigen present on the aberrant population of cells; (d) expanding the population of the orthogonal receptor expressing CAR-T cells ex vivo with an IL2 ortholog; (e) administering a pharmaceutically effective amount of the orthogonal receptor expressing CAR-T cells to the mammal; and (f) modulating the growth of the orthogonal CD122 receptor expressing CAR-T cells by the administration of a therapeutically effective amount of an IL2 ortholog that binds selectively to the orthogonal CD122 receptor expressed on the CAR-T cell. In one embodiment, the foregoing method is associated with lymphodepletion or immunosuppression of the mammal prior to the initiation of the course of CAR-T cell therapy. In another embodiment, the foregoing method is practiced in the absence of lymphodepletion and/or immunosuppression of the mammal.

### Therapeutic Combinations:

The engineered immune cells of the present disclosure may be combined with additional therapeutic agents. For example, when the disease, disorder or condition to be treated is a neoplastic disease (e.g. cancer) the methods of the present disclosure may be combined with conventional chemotherapeutic agents or other biological anti-cancer drugs such as checkpoint inhibitors (e.g. PD1 or PDL1 inhibitors) or therapeutic monoclonal antibodies (e.g. Avastin, Herceptin).

Examples of chemical agents useful in combination with the engineered immune cells of the present disclosure useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

The engineered immune cells of the present disclosure may be be administered in combination with one or more additional therapeutic agents selected from the group consisting of tyrosine-kinase inhibitors, such as Imatinib mesylate (marketed as Gleevec®, also known as STI-571), Gefitinib (Iressa®, also known as ZD1839), Erlotinib (marketed as Tarceva®), Sorafenib (Nexavar®), Sunitinib (Sutent®), Dasatinib (Sprycel®), Lapatinib (Tykerb®), Nilotinib (Tasigna®), and Bortezomib (Velcade®), Jakafi® (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt®), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel®), everolimus (Afinitor®), Vemurafenib (Zelboraf®), Trametinib (Mekinist), and Dabrafenib (Tafinlar®).

The engineered immune cells of the present disclosure may be administered in combination, particularly where the engineered immune cell is a CAR directed against BCMA, the engineered CAR-T cell may be administered in combination with a γ-Secretase Inhibitor (GSI) as described in Pont, et al. (2019) “γ-secretase inhibition increases efficacy of BCMA-specific chimeric antigen receptor T cells in multiple myeloma” Blood https://doi.org/10.1182/blood.2019000050.

The engineered immune cells of the present disclosure may be administered in combination cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens readily appreciated by the skilled clinician in the art.

The engineered immune cells of the present disclosure may be administered in combination tumor specific monoclonal antibodies without limitation, Rituximab (marketed as MabThera or Rituxan), Alemtuzumab, Panitumumab, Ipilimumab (Yervoy), etc.

The invention now being fully described, it will be apparent to one of ordinary skill in the art that various changes and modifications can be made without departing from the spirit or scope of the invention.

1. A mammalian cell comprising: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

2. The mammalian cell of claim 1 wherein the cell is an immune cell.

3. The immune cell of claim 2 selected from the group consisting of B cells, T cells, Natural Killer (NK) cells, NKT cells, cytotoxic T lymphocytes (CTLs), regulatory T cells (Tregs), dendritic cells, killer dendritic cells, and mast cells. inflammatory T-lymphocytes, cytotoxic T-lymphocytes, regulatory T-lymphocytes or helper T-lymphocytes including tumor infiltrating lymphocytes (TILs), CD4+ T-lymphocytes and CD8+ T-lymphocytes, cytotoxic T lymphocytes (CTLs), a regulatory T cell (Tregs), including subsets of CD8+ T lymphocytes of various phenotypes including T effector memory phenotype (Tem), T central memory phenotype (Tcm), terminally differentiated Tcm and Tem cells that express CD45RA (Temra), tissue resident memory (Trm) cells, and peripheral memory (Tpm) cells.

4. The immune cell of claim 2 wherein the cell is targeting redirected immune cell.

5. The targeting redirected immune cell of claim 4 selected from the group consisting CAR-T cells and TCR-engineered cells.

6. The targeting redirected immune cell of claim 5 wherein the cell is is a CAR-T.

7. The CAR-T cell of claim 6 wherein the targeting domain of the CAR-T cell exhibits specific binding to one or more tumor antigens selected from the group consisting of CD19, CD20, CD22, ROR1, CD4, CD7, CD38, CD30, B-cell maturation antigen, Lewis Y antigen, mesothelin, EGFR, GPC3, MUC1, HER2, GD2, CEA, EpCAM, PSCA, PSMA, IL3Ra2, EGFRvIII, CAIX, c-Met, and TAG72.

8. The immune cell of claim 2 wherein recombinantly modified immune cell is further modified to express at least one drug resistance gene, the drug resistance gene operably linked to an expression control sequence operable in the immune cell.

9. A recombinant vector encoding: (a) a first nucleic acid sequence encoding a receptor polypeptide, the receptor polypeptide comprising an intracellular domain, a transmembrane domain, and an extracellular domain, wherein the extracellular domain of said receptor comprises a polypeptide of the FORMULA #1 and (b) a second nucleic acid sequence encoding a ligand polypeptide that specifically binds to the extracellular domain of the receptor polypeptide, the ligand polypeptide having an amino acid sequence of the FORMULA #2.

10. The vector of claim 9 wherein the first and second nucleic acid sequences are operably linked to an expression control sequence operable in the target recombinantly modified immune cell such that expression of the first and second nucleic acid sequences are under control of a single expression control sequence.

11. The vector of claim 10 the first and second nucleic acid sequences are linked by nucleic acid sequence corresponding to mRNA a ribosome skipping site

12. The recombinant vector of claim 9 wherein vector further encodes a CAR.

13. The recombinant vector of claim 12 wherein the nucleic acid sequence encoding the orthogonal receptor and the orthogonal ligand are under the control of a single expression control sequence.

14. The recombinant vector of claim 12 wherein the first and second nucleic acid sequence are operably linked to individual expression control sequences, such sequences each being operable in the recombinantly modified immune cell such that expression of the first and second nucleic acid sequences are under control of a separate expression control sequence.

15. The recombinant vector of claim 12 wherein the expression control sequence is selected from the group consisting of constitutively active, selectively active, and regulated expression control sequences a.

16. The vector of claim 9 wherein the expression vector is viral vector selected from the group consisting of is a gamma retrovirus a self-inactivating lentiviral and retroviral

17. A recombinantly modified cell expressing an orthogonal receptor, the orthogonal receptor having an extracellular domain that specifically binds to a cognate orthogonal ligand, a transmembrane domain and an intracellular domain.

18. The cell of claim 17 wherein the orthogonal receptor comprises an ECD having an amino acid sequence of SEQ ID NO. 1.

19. The cell of claim 17 wherein the orthogonal receptor comprises an ECD having an amino acid sequence of SEQ ID NO. 1.

20. The cell of claim 17 wherein the orthogonal receptor comprises an ECD having an amino acid sequence of SEQ ID NO. 1.

21.-23. (canceled)


SEQUENCE LISTING (60 sequences)

SEQ ID NO: 1 - protein, 214 aa, Artificial Sequence
  Description of Artificial Sequence: Synthetic polypeptide
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDT

SEQ ID NO: 2 - protein, 524 aa, Artificial Sequence
  Description of Artificial Sequence: Synthetic polypeptide
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHL

SEQ ID NO: 3-4 - skipped (2 entries)

SEQ ID NO: 5 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 6 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 7 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 8 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 9 - protein, 133 aa, Homo sapiens [sequence omitted]

SEQ ID NO: 10 - skipped

SEQ ID NO: 11 - protein, 4 aa, Artificial Sequence
  Description of Artificial Sequence: Synthetic peptide
YLRQ

SEQ ID NO: 12 - protein, 4 aa, Artificial Sequence
  Description of Artificial Sequence: Synthetic peptide
YLKQ

SEQ ID NO: 13 - protein, 4 aa, Artificial Sequence
  Description of Artificial Sequence: Synthetic peptide
YRHQ

SEQ ID NO: 14 - protein, 4 aa, Artificial Sequence
  Description of Artificial Sequence: Synthetic peptide
YLRQ

SEQ ID NO: 15 - protein, 4 aa, Artificial Sequence
  Description of Artificial Sequence: Synthetic peptide
YFKQ

SEQ ID NO: 16 - protein, 4 aa, Artificial Sequence
  Description of Artificial Sequence: Synthetic peptide
YLPQ

SEQ ID NO: 17 - protein, 4 aa, Artificial Sequence
  Description of Artificial Sequence: Synthetic peptide
YMPQ

SEQ ID NO: 18 - protein, 5 aa, Artificial Sequence
  Description of Artificial Sequence: Synthetic peptide
YDKPH

SEQ ID NO: 19 - protein, 462 aa, Artificial Sequence
  Description of Artificial Sequence: Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPANNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 20 - DNA, 1389 bp, Artificial Sequence
  Description of Artificial Sequence: Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 21 - protein, 498 aa, Artificial Sequence
  Description of Artificial Sequence: Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 22 - DNA, 1496 bp, Artificial Sequence
  Description of Artificial Sequence: Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcacagagacaaggccctctgatcccaccctgggggtggccaggcaacacccttgttgctgtgtccatctttctcctgctgactggcccgacctacctcctgttcaagctgtcgcccagggtgaagagaatcttctaccagaacgtgccctctccagcgatgttcttccagcccctctacagtgtacacaatgggaacttccagacttggatgggggcccacggggccggtgtgctgttgagccaggactgtgctggcaccccacagggagccttggagccctgcgtccaggaggccactgcactgctcacttgtggcccagcgcgtccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattcta

SEQ ID NO: 23 - protein, 545 aa, Artificial Sequence
  Description of Artificial Sequence: Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 24 - DNA, 1638 bp, Artificial Sequence
  Description of Artificial Sequence: Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 25 - protein, 497 aa, Artificial Sequence
  Description of Artificial Sequence: Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPASDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 26 - DNA, 1494 bp, Artificial Sequence
  Description of Artificial Sequence: Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag

SEQ ID NO: 27 - DNA, 1656 bp, Artificial Sequence
  Description of Artificial Sequence: Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccattccgtggctcggccacctcctcgtgggtctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 28 - protein, 11 aa, Artificial Sequence
  Description of Artificial Sequence: Synthetic peptide
DICLPRWGCLW

SEQ ID NO: 29 - protein, 22 aa, Artificial Sequence
  Description of Artificial Sequence: Synthetic peptide
GSGATNFSLLKQAGDVEENPGP

SEQ ID NO: 30 - protein, 219 aa, Artificial Sequence
  Description of Artificial Sequence: Synthetic polypeptide
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 31 - protein, 132 aa, Artificial Sequence
  Description of Artificial Sequence: Synthetic polypeptide
PTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCHSIISTLT

SEQ ID NO: 32 - protein, 132 aa, Artificial Sequence
  Description of Artificial Sequence: Synthetic polypeptide
PTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 33 - protein, 4 aa, Artificial Sequence
  Description of Artificial Sequence: Synthetic peptide
  MOD_RES (2)..(2): Any natural amino acid
  MOD_RES (3)..(3): Any natural amino acid
YXXL

SEQ ID NO: 34 - protein, 4 aa, Artificial Sequence
  Description of Artificial Sequence: Synthetic peptide
YLSL

SEQ ID NO: 35 - protein, 4 aa, Artificial Sequence
  Description of Artificial Sequence: Synthetic peptide
  MOD_RES (2)..(2): L, R, F or M
  MOD_RES (3)..(3): R, K, H or P
YXXQ

SEQ ID NO: 36 - protein, 214 aa, Artificial Sequence
  Description of Artificial Sequence: Synthetic polypeptide
  MOD_RES (70)..(70): Q or Y
  MOD_RES (73)..(73): T, D or Y
  MOD_RES (133)..(133): H, D, E or K
  MOD_RES (134)..(134): Y, F, E or R
AVNGTSQFTCFYNSRANISCVWSQDGALQDTSCQVHAWPDRRRWNQTCELLPVSQASWACNLILGAPDSXKLXTVDIVTLRVLCREGVRWRVMAIQDFKPFENLRLMAPISLQVVHVETHRCNISWEISQASXXFERHLEFEARTLSPGHTWEEAPLLTLKQKQEWICLETLTPDTQYEFQVRVKPLQGEFTTWSPWSQPLAFRTKPAALGKDT

SEQ ID NO: 37 - protein, 133 aa, Artificial Sequence
  Description of Artificial Sequence: Synthetic polypeptide
  MOD_RES (1)..(1): A or absent
  MOD_RES (2)..(2): P or absent
  MOD_RES (3)..(3): T, C, A, G, Q, E, N, D, R, K, P, or absent
  MOD_RES (4)..(4): S or absent
  MOD_RES (5)..(5): S or absent
  MOD_RES (6)..(6): S or absent
  MOD_RES (7)..(7): T or absent
  MOD_RES (8)..(8): K or absent
  MOD_RES (9)..(9): K or absent
  MOD_RES (13)..(13): Q, W or absent
  MOD_RES (14)..(14): L, M, W or absent
  MOD_RES (15)..(15): E, K, D, T, A, S, Q, H or absent
  MOD_RES (16)..(16): H, N or Q or absent
  MOD_RES (18)..(18): L, R, G, M, F, E, H, W, K, Q, S, V, I, Y, H, D or T
  MOD_RES (19)..(19): L, A, V, I or absent
  MOD_RES (20)..(20): D, T, S, M, L, or absent
  MOD_RES (22)..(22): Q, F, E, G, A, L, M, F, W, K, S, V, I, Y, H, R, N, D, T, F or absent
  MOD_RES (23)..(23): M, A, W, H, Y, F, Q, S, V, L, T, or absent
  MOD_RES (27)..(27): G, K, S or absent
  MOD_RES (38)..(38): R, W or G
  MOD_RES (39)..(39): M, L or V
  MOD_RES (42)..(42): F or K
  MOD_RES (51)..(51): T, I or absent
  MOD_RES (55)..(55): H or Y
  MOD_RES (74)..(74): Q, N, H, S
  MOD_RES (80)..(80): L, F or V
  MOD_RES (81)..(81): R, I, D, Y, T or absent
  MOD_RES (85)..(85): L or V
  MOD_RES (86)..(86): I or V
  MOD_RES (88)..(88): N, E or Q or absent
  MOD_RES (89)..(89): I or V
  MOD_RES (91)..(91): V, R or K
  MOD_RES (92)..(92): I or F
  MOD_RES (97)..(97): K or Q
  MOD_RES (104)..(104): M or A
  MOD_RES (109)..(109): D, C or a non-natural amino acid with an activated side chain
  MOD_RES (113)..(113): T or N
  MOD_RES (125)..(125): C, A or S
  MOD_RES (126)..(126): Q, H, M, K, C, D, E, G, I, R, S, or T
  MOD_RES (130)..(130): S, T or R
XXXXXXXXXTQLXXXXLXXXLXXILNXINNYKNPKLTXXLTXKFYMPKKAXELKXLQCLEEELKPLEEVLNLAXSKNFHXXPRDXXSXXNXXVLELXGSETTFXCEYAXETAXIVEFLNRWITFXXSIIXTLT

SEQ ID NO: 38 - protein, 22 aa, Artificial Sequence
  Description of Artificial Sequence: Synthetic peptide
  MOD_RES (22)..(22): Any amino acid
GSGEGRGSLLTCGDVEENPGPX

SEQ ID NO: 39 - protein, 23 aa, Artificial Sequence
  Description of Artificial Sequence: Synthetic peptide
GSGQCTNYALLKLAGDVESNPGP

SEQ ID NO: 40 - protein, 5 aa, Artificial Sequence
  Description of Artificial Sequence: Synthetic peptide
GGGGS

SEQ ID NO: 41 - protein, 42 aa, Homo sapiens
TACDLAPPAGTTDAAHPGRSVVPALLPLLAGTLLLLETATAP

SEQ ID NO: 42 - protein, 41 aa, Homo sapiens
HETTPNKGSGTTSGTTRLLSGHTCFTLTGLLGTLVTMGLLT

SEQ ID NO: 43 - protein, 35 aa, Trypanosoma brucei
EPEPEPEPEPEPGAATLKSVALPFAIAAAALVAAF

SEQ ID NO: 44 - protein, 36 aa, Mesocricetus sp.
QKESQAYYDGRRSSAVLFSSPPVILLISFLIFLMVG

SEQ ID NO: 45 - protein, 44 aa, Rattus sp.
KTINVIRDKLVKCGGISLLVQNTSWLLLLLLSLSFLQATDFISI

SEQ ID NO: 46 - protein, 36 aa, Trypanosoma brucei
ESNCKWENNACKDSSILVTKKFALTVVSAAFVALLF

SEQ ID NO: 47 - protein, 38 aa, Torpedo marmorata
NQFLPKLLNATACSSSGTSSSKGIIFYVLFSILYLIFY

SEQ ID NO: 48 - protein, 6 aa, Artificial Sequence
  Description of Artificial Sequence: Synthetic His tag
  SITE (1)..(6): This sequence may encompass 3-6 residues
HHHHHH

SEQ ID NO: 49 - protein, 6 aa, Artificial Sequence
  Description of Artificial Sequence: Synthetic 6xHis tag
HHHHHH

SEQ ID NO: 50 - protein, 6 aa, Artificial Sequence
  Description of Artificial Sequence: Synthetic peptide
GGYLRQ

SEQ ID NO: 51 - protein, 6 aa, Artificial Sequence
  Description of Artificial Sequence: Synthetic peptide
GGYLKQ

SEQ ID NO: 52 - protein, 6 aa, Artificial Sequence
  Description of Artificial Sequence: Synthetic peptide
GGYRHQ

SEQ ID NO: 53 - protein, 6 aa, Artificial Sequence
  Description of Artificial Sequence: Synthetic peptide
GGYFKQ

SEQ ID NO: 54 - protein, 6 aa, Artificial Sequence
  Description of Artificial Sequence: Synthetic peptide
GGYLPQ

SEQ ID NO: 55 - protein, 6 aa, Artificial Sequence
  Description of Artificial Sequence: Synthetic peptide
GGYMPQ

SEQ ID NO: 56 - protein, 7 aa, Artificial Sequence
  Description of Artificial Sequence: Synthetic peptide
GGYDKPH

SEQ ID NO: 57 - protein, 10 aa, Artificial Sequence
  Description of Artificial Sequence: Synthetic peptide
YFTYDPYSEE

SEQ ID NO: 58 - protein, 4 aa, Artificial Sequence
  Description of Artificial Sequence: Synthetic peptide
YFTY

SEQ ID NO: 59 - protein, 4 aa, Artificial Sequence
  Description of Artificial Sequence: Synthetic peptide
YDPY

SEQ ID NO: 60 - protein, 4 aa, Artificial Sequence
  Description of Artificial Sequence: Synthetic peptide
YSEE